(19) United States
(12) Patent Application Publication
(10) Pub. No.: US 2020/0000697 A1
(43) Pub. Date: Jan. 2, 2020

(54) APTAMERS FOR HAIR CARE APPLICATIONS

(71) Applicant: The Procter & Gamble Company, Cincinnati, OH (US)

(72) Inventors: Juan Esteban Velasquez, Cincinnati, OH (US); Amy Violet Trejo, Oregonia, OH (US); Jennifer Mary Marsh, Deerfield Township, OH (US); Gregory Allen Penner, London (CA)

(21) Appl. No.: 16/270,911

(22) Filed: Feb. 8, 2019

Related U.S. Application Data
(60) Provisional application No. 62/692,068, filed on Jun. 29, 2018.

(51) Int. Cl. A61K 8/60; A61Q 5/12

(52) U.S. Cl. CPC A61K 8/606; C12N 15/115; A61Q 5/12

(57) ABSTRACT

The present invention is directed to an aptamer composition comprising at least one oligonucleotide consisting of: deoxyribonucleotides, ribonucleotides, derivatives of deoxyribonucleotides, derivatives of ribonucleotides, and mixtures thereof; wherein said aptamer composition has a binding affinity for a material selected from the group consisting of: undamaged hair, damaged hair, hair cuticle, hair epicuticle, hair exocuticle, hair endocuticle, hair cortex, hair keratins, hair F-layer, hair lipids, 18-methyleicosanoic acid, and mixtures thereof.

### FIELD OF INVENTION

The present invention generally relates to nucleic acid aptamers that have a high binding affinity and specificity for damaged human hair. This invention also relates to the use of such aptamers as delivery vehicles of active ingredients to the hair.

### BACKGROUND OF THE INVENTION

Aptamers are short single-stranded oligonucleotides, with a specific and complex three-dimensional shape, that bind to target molecules. The molecular recognition of aptamers is based on structure compatibility and intermolecular interactions, including electrostatic forces, van der Waals interactions, hydrogen bonding, and π-π stacking interactions of aromatic rings with the target material. The targets of aptamers include, but are not limited to, peptides, proteins, nucleotides, amino acids, antibiotics, low molecular weight organic or inorganic compounds, and even whole cells. The dissociation constant of aptamers typically varies between micromolar and picomolar levels, which is comparable to the affinity of antibodies to their antigens. Aptamers can also be designed to have high specificity, enabling the discrimination of target molecules from closely related derivatives.

Aptamers are usually designed in vitro from large libraries of random nucleic acids by Systematic Evolution of Ligands by Exponential Enrichment (SELEX). The SELEX method is first introduced in 1990 when single stranded RNAs are selected against low molecular weight dyes (Ellington, A. D., Szostak, J. W., 1990. Nature 346: 818-822). A few years later, single stranded DNA aptamers and aptamers containing chemically modified nucleotides are also described (Ellington, A. D., Szostak, J. W., 1992. Nature 355: 850-852; Green, L. S., et al., 1995. Chem. Biol. 2: 683-695). Since then, aptamers for hundreds of microscopic targets, such as cations, small molecules, proteins, cells, or tissues have been selected. A compilation of examples from the literature is included in the database at the website: http://www.aptagen.com/aptamer-index/aptamer-list.aspx. However, a need still exists for aptamers that selectively bind to hair, including damaged hair.

### SUMMARY OF THE INVENTION

In this invention, we have demonstrated the use of SELEX for the selection of aptamers against damaged hair and the use of such aptamers for the delivery of active ingredients to the hair.

In the present invention, an aptamer composition is provided. The aptamer composition comprises at least one oligonucleotide consisting of: deoxyribonucleotides, ribonucleotides, derivatives of deoxyribonucleotides, derivatives of ribonucleotides, and mixtures thereof; wherein said aptamer composition has a binding affinity for a material selected from the group consisting of: undamaged hair, damaged hair, hair cuticle, hair epicuticle, hair exocuticle, hair endocuticle, hair cortex, hair keratins, hair F-layer, hair lipids, 18-methyleicosanoic acid, and mixtures thereof.

In the present invention, an aptamer composition is provided. The aptamer composition of claim 1, may comprise at least one oligonucleotide selected from the group consisting of SEQ ID NO 1, SEQ ID NO 2, SEQ ID NO 101, SEQ ID NO 102, SEQ ID NO 214 to SEQ ID NO 220.

In the present invention, the aptamer composition may comprise at least one oligonucleotide comprising one or more motifs selected from the group consisting of SEQ ID NO 201 to SEQ ID NO 213.

In the present invention, a hair care composition is provided. The hair care composition may comprise at least one nucleic acid aptamer; wherein said at least one nucleic acid aptamer has a binding affinity for a hair component. In the present invention, wherein said hair component is selected from the group comprising: hair cuticle, hair epicuticle, hair exocuticle, hair endocuticle, hair cortex, hair keratins, hair F-layer, hair lipids, 18-methyleicosanoic acid, and mixtures thereof.

In the present invention, a method for delivering one or more hair care active ingredients to the hair is provided. The method may comprise administering a hair care composition comprising at least one nucleic acid aptamer and one or more hair care active ingredients; wherein said at least one nucleic acid aptamer and said one or more hair care active ingredients are covalently or non-covalently attached; and wherein said at least one nucleic acid aptamer has a binding affinity for a hair component.

In the present invention, a method for delivering one or more hair care active ingredients to the hair is provided. The method may comprise administering a hair care composition comprising: at least one nucleic acid aptamer and one or more nanomaterials; wherein said at least one nucleic acid aptamer and said one or more nanomaterials are covalently or non-covalently attached; and wherein said at least one nucleic acid aptamer has a binding affinity for a hair component.

### BRIEF DESCRIPTION OF THE DRAWING FIGURES

For a more complete understanding of the disclosure, reference should be made to the following detailed description and drawing Figures.

FIG. 1. Aptamer selection strategy.

FIG. 2. Total number of sequences on each selection library.

FIG. 3. The enrichment trajectories of the top 20 sequences in terms frequency across different selection rounds for channel A.

FIG. 4. The enrichment trajectories of the top 20 sequences in terms of frequency across different selection rounds for channel B.

FIG. 5. Correlation matrix ordered by clustering (Ward.D2 method) for enrichment trajectories of top 100 aptamers of channel A.

FIG. 6. Correlation matrix ordered by clustering (Ward.D2 method) for enrichment trajectories of top 100 aptamers of channel B.

FIG. 7. Binding of different aptamers at 50 nM to different hair samples.

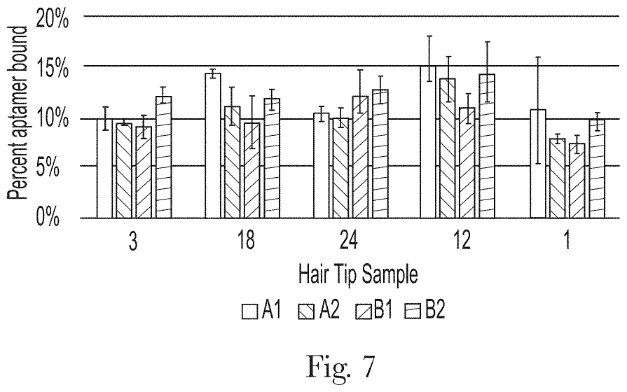

FIG. 8. Effect of concentration of aptamers on the total amount bound to hair sample 1.

FIG. 9. Effect of concentration of aptamers on the percentage bound to hair sample 1.

FIG. 10. Effect of hair type (root versus tip) on the percentage of aptamer bound to hair sample #18.

FIG. 11. Motif analysis of random region of aptamer H-A1.

FIG. 12. The predicted secondary structures of aptamer H-A1 and its conserved motif.

FIG. 13. Motif analysis of random region of aptamer H-A2.

FIG. 14. The predicted secondary structures of aptamer H-A2 and its conserved motif.

FIG. 15. Motif analysis of random region of aptamer H-B1.

FIG. 16. The predicted secondary structures of aptamer H-B1 and its conserved motifs.

FIG. 17. Motif analysis of random region of aptamer H-B2.

FIG. 18. The predicted secondary structures of aptamer H-B2 and its conserved motif.

FIG. 19. Alignment of exemplary sequences with at least 50% nucleotide sequence identity that are identified during the selection process.

FIG. 20. Predicted secondary structures of truncated aptamers H-A1.1 (left) and H-A1.2 (right). The conserved motif (SEQ ID NO 201) is highlighted.

FIG. 21. Predicted secondary structures of truncated aptamers H-A2.1 (left) and H-A2.2 (right).

FIG. 22. Predicted secondary structures of truncated aptamers H-B1.1 (left) and H-B1.2 (right). The conserved motifs (SEQ ID NO 204 and SEQ ID NO 205) are highlighted.

FIG. 23. Predicted secondary structure of truncated aptamers H-B2.1. The conserved motif (SEQ ID NO 212) is highlighted.

### DETAILED DESCRIPTION OF THE INVENTION

### I Definitions

As used herein, the term “aptamer” refers to a single stranded oligonucleotide or a peptide that has a binding affinity for a specific target.

As used herein, the term “nucleic acid” refers to a polymer or oligomer of nucleotides. Nucleic acids are also referred as “ribonucleic acids” when the sugar moiety of the nucleotides is D-ribose and as “deoxyribonucleic acids” when the sugar moiety is 2-deoxy-D-ribose.

As used herein, the term “nucleotide” usually refers to a compound consisting of a nucleoside esterified to a monophosphate, polyphosphate, or phosphate-derivative group via the hydroxyl group of the 5-carbon of the sugar moiety. Nucleotides are also referred as “ribonucleotides” when the sugar moiety is D-ribose and as “deoxyribonucleotides” when the sugar moiety is 2-deoxy-D-ribose.

As used herein, the term “nucleoside” refers to a glycosylamine consisting of a nucleobase, such as a purine or pyrimidine, usually linked to a 5-carbon sugar (e.g. D-ribose or 2-deoxy-D-ribose) via a β-glycosidic linkage. Nucleosides are also referred as “ribonucleosides” when the sugar moiety is D-ribose and as “deoxyribonucleosides” when the sugar moiety is 2-deoxy-D-ribose.

As used herein, the term “nucleobase”, refers to a compound containing a nitrogen atom that has the chemical properties of a base. Non-limiting examples of nucleobases are compounds comprising pyridine, purine, or pyrimidine moieties, including, but not limited to adenine, guanine, hypoxanthine, thymine, cytosine, and uracil.

As used herein, the term “oligonucleotide” refers to an oligomer composed of nucleotides.

As used herein, the term “identical” or “sequence identity,” in the context of two or more oligonucleotides, nucleic acids, or aptamers, refers to two or more sequences that are the same or have a specified percentage of nucleotides that are the same, when compared and aligned for maximum correspondence, as measured using sequence comparison algorithms or by visual inspection.

As used herein, the term “substantially homologous” or “substantially identical” in the context of two or more oligonucleotides, nucleic acids, or aptamers, generally refers to two or more sequences or subsequences that have at least 40%, 60%, 80%, 90%, 95%, 96%, 97%, 98% or 99% nucleotide identity, when compared and aligned for maximum correspondence, as measured using sequence comparison algorithms or by visual inspection.

As used herein, the term “epitope” refers to the region of a target that interacts with the aptamer. An epitope can be a contiguous stretch within the target or can be represented by multiple points that are physically proximal in a folded form of the target.

As used herein, the term “motif” refers to the sequence of contiguous, or series of contiguous, nucleotides occurring in a library of aptamers with binding affinity towards a specific target (e.g hair) and that exhibits a statistically significant higher probability of occurrence than would be expected compared to a library of random oligonucleotides. The motif sequence is frequently the result or driver of the aptamer selection process.

As used herein, the term “Damaged hair” is hair that has been exposed to (a) a chemical treatment, such as permanent or semi-permanent coloring, permanent or semi-permanent styling, relaxers, bleaching, etc, (b) mechanically damage from repeated use of brushing or combing, (c) thermal damage from use of hair dryers and/or hot implements such as flat iron, and (d) environmental exposure to UV sunlight, bleached water, etc.

It is well known that the natural outer hair layer (F-layer) is partially or totally removed by chemical treatments or exposure to environmental factors, making the hair fibers more hydrophilic Thus, the natural weatherproofing, while helping to seal in moisture and prevent further damage is removed, making the hair more prone to further chemical and/or mechanical damage.

As used herein, the term “Non-damaged hair”, “virgin hair”, is hair in its natural state that has not been significantly exposed to the above-mentioned conditions. Virgin hair can be collected from people who do not use chemical treatments, heating implements, excessive brushing or significant exposure to UV light, bleached water, etc. Also, consumers' newly emerged hair (roots) have more characteristics of virgin hair than the ends of the hair as they have less exposure to the above-mentioned conditions that damage the hair.

As used herein, the term “binding affinity” refers to:

Binding affinity=Amount of aptamer bound to the hair sample/Total amount of aptamer incubated with the hair sample×100%.

The higher the amount of aptamer bound to the hair sample, the higher the binding affinity under the tested conditions.

### II. Aptamer Compositions

Nucleic acid aptamers are single-stranded oligonucleotides, with specific secondary and tertiary structures, that can bind to targets with high affinity and specificity. In the present invention, an aptamer composition may comprise at least one oligonucleotide consisting of: deoxyribonucleotides, ribonucleotides, derivatives of deoxyribonucleotides, derivatives of ribonucleotides, and mixtures thereof; wherein said aptamer composition has a binding affinity for a material selected from the group consisting of: undamaged hair, damaged hair, hair cuticle, hair epicuticle, hair exocuticle, hair endocuticle, hair cortex, hair keratins, hair F-layer, hair lipids, 18-methyleicosanoic acid, and mixtures thereof. In the present invention, said aptamer composition may have a binding affinity for damaged hair. In the present invention, said aptamer composition may have a higher binding affinity for damaged hair than for undamaged hair.

In the present invention, said aptamer composition may comprise at least one oligonucleotide selected from the group consisting of oligonucleotides with at least 50% nucleotide sequence identity to sequences selected from the group consisting of SEQ ID NO 1 to SEQ ID NO 200 and SEQ ID NO 213 to SEQ ID NO 219. In the present invention, said aptamer composition may comprise at least one oligonucleotide selected from the group consisting of oligonucleotides with at least 70% nucleotide sequence identity to sequences selected from the group consisting of SEQ ID NO 1 to SEQ ID NO 200 and SEQ ID NO 213 to SEQ ID NO 219. In the present invention, said aptamer composition may comprise at least one oligonucleotide selected from the group consisting of oligonucleotides with at least 90% nucleotide sequence identity to sequences selected from the group consisting of SEQ ID NO 1 to SEQ ID NO 200 and SEQ ID NO 213 to SEQ ID NO 219. In the present invention, said aptamer composition may comprises at least one oligonucleotide selected from the group consisting of SEQ ID NO 1 to SEQ ID NO 200 and SEQ ID NO 213 to SEQ ID NO 219.

In the present invention, said aptamer composition may comprise at least one oligonucleotide selected from the group consisting of oligonucleotides containing at least 10 contiguous nucleotides from sequences selected from the group consisting of SEQ ID NO 1 to SEQ ID NO 200. In the present invention, said aptamer composition may comprise at least one oligonucleotide selected from the group consisting of oligonucleotides containing at least 20 contiguous nucleotides from sequences selected from the group consisting of SEQ ID NO 1 to SEQ ID NO 200. In the present invention, said aptamer composition may comprise at least one oligonucleotide selected from the group consisting of oligonucleotides containing at least 30 contiguous nucleotides from sequences selected from the group consisting of SEQ ID NO 1 to SEQ ID NO 200. In the resent invention, said aptamer composition may comprise at least one oligonucleotide selected from the group consisting of oligonucleotides containing at least 40 contiguous nucleotides from sequences selected from the group consisting of SEQ ID NO 1 to SEQ ID NO 200. In the present invention, said aptamer composition may comprise at least one oligonucleotide selected from the group consisting of oligonucleotides containing at least 60 contiguous nucleotides from sequences selected from the group consisting of SEQ ID NO 1 to SEQ ID NO 200. In the present invention, said aptamer composition may comprise at least one oligonucleotide selected from the group consisting of oligonucleotides containing at least 70 contiguous nucleotides from sequences selected from the group consisting of SEQ ID NO 1 to SEQ ID NO 200. A non-limiting example of oligonucleotide containing at least 30 contiguous nucleotides from SEQ ID NO 1 is SEQ ID NO 213. A non-limiting example of oligonucleotide containing at least 20 contiguous nucleotides from SEQ ID NO 1 is SEQ ID NO 214. A non-limiting example of oligonucleotide containing at least 20 contiguous nucleotides from SEQ ID NO 2 is SEQ ID NO 215. A non-limiting example of oligonucleotide containing at least 30 contiguous nucleotides from SEQ ID NO 2 is SEQ ID NO 216. A non-limiting example of oligonucleotide containing at least 30 contiguous nucleotides from SEQ ID NO 101 is SEQ ID NO 217. A non-limiting example of oligonucleotide containing at least 20 contiguous nucleotides from SEQ ID NO 101 is SEQ ID NO 218. A non-limiting example of oligonucleotide containing at least 40 contiguous nucleotides from SEQ ID NO 102 is SEQ ID NO 219.

In the present invention, said aptamer composition may comprise at least one oligonucleotide selected from the group consisting of SEQ ID NO 1, SEQ ID NO 2, SEQ ID NO 101, SEQ ID NO 102, and SEQ ID NO 213 to SEQ ID NO 219. In the present invention, said aptamer composition may comprise at least one oligonucleotide selected from the group consisting of oligonucleotides with at least 50% nucleotide sequence identity to sequences selected from the group consisting of SEQ ID NO 1, SEQ ID NO 2, SEQ ID NO 101, SEQ ID NO 102, and SEQ ID NO 213 to SEQ ID NO 219. In the present invention, said aptamer composition may comprise at least one oligonucleotide selected from the group consisting of oligonucleotides with at least 60% nucleotide sequence identity to sequences selected from the group consisting of SEQ ID NO 1, SEQ ID NO 2, SEQ ID NO 101, SEQ ID NO 102, and SEQ ID NO 213 to SEQ ID NO 219. In the present invention, said aptamer composition may comprise at least one oligonucleotide selected from the group consisting of oligonucleotides with at least 70% nucleotide sequence identity to sequences selected from the group consisting of SEQ ID NO 1, SEQ ID NO 2, SEQ ID NO 101, SEQ ID NO 102, and SEQ ID NO 213 to SEQ ID NO 219. In the present invention, said aptamer composition may comprise at least one oligonucleotide selected from the group consisting of oligonucleotides with at least 90% nucleotide sequence identity to sequences selected from the group consisting of SEQ ID NO 1, SEQ ID NO 2, SEQ ID NO 101, SEQ ID NO 102, and SEQ ID NO 213 to SEQ ID NO 219. A non-limiting example of an oligonucleotide with at least 60% nucleotide sequence identity to SEQ ID NO 8 is SEQ ID NO 79. A non-limiting example of an oligonucleotide with at least 60% nucleotide sequence identity to SEQ ID NO 46 is SEQ ID NO 156. A non-limiting example of an oligonucleotide with at least 50% nucleotide sequence identity to SEQ ID NO 52 is SEQ ID NO 53.

In the present invention, wherein said at least one oligonucleotide may comprise one or more motifs selected from the group consisting of SEQ ID NO 201 to SEQ ID NO 212. In the present invention, said aptamer composition may comprise at least one oligonucleotide comprising a sequence of nucleotides with at least 70% nucleotide sequence identity to sequences selected from the group consisting of SEQ ID NO 201 to SEQ ID NO 212. In the present invention, said aptamer composition may comprise at least one oligonucleotide comprising a sequence of nucleotides with at least 80% nucleotide sequence identity to sequences selected from the group consisting of SEQ ID NO 201 to SEQ ID NO 212. In the present invention, said aptamer composition may comprise at least one oligonucleotide comprising a sequence of nucleotides with at least 90% nucleotide sequence identity to sequences selected from the group consisting of SEQ ID NO 201 to SEQ ID NO 212.

Chemical modifications can introduce new features into the aptamers such as different molecular interactions with the target, improved binding capabilities, enhanced stability of oligonucleotide conformations, or increased resistance to nucleases. In the present invention, said at least one oligonucleotide of said aptamer composition may comprise natural or non-natural nucleobases. Natural nucleobases are adenine, cytosine, guanine, thymine, and uracil. Non-limiting examples of non-natural nucleobases are hypoxanthine, xanthine, 7-methylguanine, 5,6-dihydrouracil, 5-5-methylcytosine, 5-hydroxymethylcytosine, thiouracil, 1-methylhypoxanthine, 6-methylisoquinoline-1-thione-2-yl, 3-methoxy-2-naphthyl, 5-propynyluracil-1-yl, 5-methylcytosin-1-yl, 2-aminoadenin-9-yl, 7-deaza-7-iodoadenin-9-yl, 7-deaza-7-propynyl-2-aminoadenin-9-yl, phenoxazinyl, phenoxazinyl-G-clam, bromouracil, 5-iodouracil, and mixtures thereof.

Modifications of the phosphate backbone of the oligonucleotides can also increase the resistance against nuclease digestion. In the present invention, the nucleosides of said oligonucleotides may be linked by a chemical motif selected from the group comprising: natural phosphate diester, chiral phosphorothionate, chiral methyl phosphonate, chiral phosphoramidate, chiral phosphate chiral triester, chiral boranophosphate, chiral phosphoroselenoate, phosphorodithioate, phosphorothionate amidate, methylenemethylimino, 3′-amide, 3′ achiral phosphoramidate, 3′ achiral methylene phosphonates, thioformacetal, thioethyl ether, fluorophosphate, and mixtures thereof. In the present invention, the nucleosides of said oligonucleotides may be linked by natural phosphate diesters.

In the present invention, the sugar moiety of the nucleosides of said oligonucleotides may be selected from the group comprising: ribose, deoxyribose, 2′-fluoro deoxyribose, 2′-O-methyl ribose, 2′-O-(3-amino)propyl ribose, 2′-O-(2-methoxy)ethyl ribose, 2′-O-2-(N,N-dimethylaminooxy)ethyl ribose, 2′-O-2-[2-(N,N-dimethylamino)ethyloxy]ethyl ribose, 2′-O-N,N-dimethylacetamidyl ribose, N-morpholinophosphordiamidate, α-deoxyribofuranosyl, other pentoses, hexoses, and mixtures thereof.

In the present invention, said derivatives of ribonucleotides or said derivatives of deoxyribonucleotides may be selected from the group comprising: locked oligonucleotides, peptide oligonucleotides, glycol oligonucleotides, threose oligonucleotides, hexitol oligonucleotides, altritol oligonucleotides, butyl oligonucleotides, L-ribonucleotides, arabino oligonucleotides, 2′-fluoroarabino oligonucleotides, cyclohexene oligonucleotides, phosphorodiamidate morpholino oligonucleotides, and mixtures thereof.

In the present invention, the nucleotides at the 5′- and 3′-ends of said at least one oligonucleotide may be inverted. In the present invention, at least one nucleotide of said at least one oligonucleotide may be fluorinated at the 2′ position of the pentose group. In present invention, the pyrimidine nucleotides of said at least one oligonucleotide may be fluorinated at the 2′ position of the pentose group. In the present invention, the aptamer composition further may comprise at least one polymeric material, wherein said at least one polymeric material is covalently linked to said at least one oligonucleotide. In the present invention, said at least one polymeric material may be polyethylene glycol.

In the present invention, said at least one oligonucleotide may between about 10 and about 200 nucleotides in length. In the present invention, said at least one oligonucleotide may be less than about 100 nucleotides in length. In the present invention, said at least one oligonucleotide may less than about 50 nucleotides in length.

In present invention, wherein said at least one oligonucleotide may be covalently or non-covalently attached to one or more hair care active ingredients. Suitable hair care active ingredients include any material that is generally considered as safe and that provides benefits to the hair, and specifically to the condition of the hair surfaces that such hair care active ingredients interact with. Examples of the hair conditions these actives address include, but are not limited to, appearance and structural changes to hair. In the present invention, said one or more hair care active ingredients may be selected from the group comprising: conditioning agents, brightening agents, strengthening agents, anti-fungal agents, anti-bacterial agents, anti-microbial agents, anti-dandruff agents, anti-malodor agents, perfumes, olfactory enhancement agents, anti-itch agents, cooling agents, anti-adherence agents, moisturization agents, smoothness agents, surface modification agents, antioxidants, natural extracts and essential oils, dyes, pigments, bleaches, nutrients, peptides, vitamins, enzymes, chelants, and mixtures thereof.

In the present invention, said at least one oligonucleotide may be non-covalently attached to said one or more hair care active ingredients via molecular interactions. Examples of molecular interactions are electrostatic forces, van der Waals interactions, hydrogen bonding, and π-π stacking interactions of aromatic rings.

In present invention, said at least one oligonucleotide may be covalently attached to said one or more hair care active ingredients using one or more linkers or spacers. Non-limiting examples of linkers are chemically labile linkers, enzyme-labile linkers, and non-cleavable linkers. Examples of chemically labile linkers are acid-cleavable linkers and disulfide linkers. Acid-cleavable linkers take advantage of low pH to trigger hydrolysis of an acid-cleavable bond, such as a hydrazone bond, to release the active ingredient or payload. Disulfide linkers can release the active ingredients under reducing environments. Examples of enzyme-labile linkers are peptide linkers that can be cleaved in the present of proteases and β-glucuronide linkers that are cleaved by glucuronidases releasing the payload. Non-cleavable linkers can also release the active ingredient if the aptamer is degraded by nucleases.

In the present invention, said at least one oligonucleotide may be covalently or non-covalently attached to one or more nanomaterials. In the present invention, said at least one oligonucleotide and said one or more hair care active ingredients may be covalently or non-covalently attached to one or more nanomaterials. In the present invention, said one or more hair care active ingredients may be carried by said one or more nanomaterials. Non-limiting examples of nanomaterials are gold nanoparticles, nano-scale iron oxides, carbon nanomaterials (such as single-walled carbon nanotubes and graphene oxide), mesoporous silica nanoparticles, quantum dots, liposomes, poly (lactide-co-glycolic acids) nanoparticles, polymeric micelles, dendrimers, serum albumin nanoparticles, and DNA-based nanomaterials. These nanomaterials can serve as carriers for large volumes of hair care active ingredients, while the aptamers can facilitate the delivery of the nanomaterials with the actives to the expected target.

Nanomaterials can have a variety of shapes or morphologies. Non-limiting examples of shapes or morphologies are spheres, rectangles, polygons, disks, toroids, cones, pyramids, rods/cylinders, and fibers. In the context of the present invention, nanomaterials usually have at least one spatial dimension that is less than about 100 μm and more preferably less than about 10 μm. Nanomaterials comprise materials in solid phase, semi-solid phase, or liquid phase.

Aptamers can also be peptides that bind to targets with high affinity and specificity. These peptide aptamers can be part of a scaffold protein. Peptide aptamers can be isolated from combinatorial libraries and improved by directed mutation or rounds of variable region mutagenesis and selection. In the present invention, an aptamer composition may comprise at least one peptide or protein; wherein said aptamer composition has a binding affinity for a material selected from the group consisting of: undamaged hair, damaged hair, hair cuticle, hair epicuticle, hair exocuticle, hair endocuticle, hair cortex, hair keratins, hair F-layer, hair lipids, 18-methyleicosanoic acid, and mixtures thereof.

### III. Methods of Designing Aptamer Compositions

The method of designing nucleic acid aptamers known as Systematic Evolution of Ligands by Exponential Enrichment (SELEX) has been broadly studied and improved for the selection of aptamers against small molecules and proteins (WO 91/19813). In brief, in the conventional version of SELEX, the process starts with the synthesis of a large library of oligonucleotides consisting of randomly generated sequences of fixed length flanked by constant 5′- and 3′-ends that serve as primers. The oligonucleotides in the library are then exposed to the target ligand and those that do not bind the target are removed. The bound sequences are eluted and amplified by PCR to prepare for subsequent rounds of selection in which the stringency of the elution conditions is usually increased to identify the tightest-binding oligonucleotides. In addition to conventional SELEX, there are improved versions such as capillary electrophoresis-SELEX, magnetic bead-based SELEX, cell-SELEX, automated SELEX, complex-target SELEX, among others. A review of aptamer screening methods is found in “Kim, Y. S. and M. B. Gu (2014). Advances in Aptamer Screening and Small Molecule Aptasensors. Adv. Biochem. Eng./Biotechnol. 140 (Biosensors based on Aptamers and Enzymes): 29-67” and “Stoltenburg, R., et al. (2007). SELEX-A (r)evolutionary method to generate high-affinity nucleic acid ligands. Biomol. Eng. 24(4): 381-403,” the contents of which are incorporated herein by reference. Although the SELEX method has been broadly applied, it is neither predictive nor standardized for every target. Instead, a method must be developed for each particular target in order for the method to lead to viable aptamers.

Despite the large number of selected aptamers, SELEX has not been routinely applied for the selection of aptamers with binding affinities towards macroscopic materials and surfaces. For the successful selection of aptamers with high binding affinity and specificity against macroscopic materials, the epitope should be present in sufficient amount and purity to minimize the enrichment of unspecifically binding oligonucleotides and to increase the specificity of the selection. Also, the presence of positively charged groups (e.g. primary amino groups), the presence of hydrogen bond donors and acceptors, and planarity (aromatic compounds) facilitate the selection of aptamers. In contrast, negatively charged molecules (e.g. containing phosphate groups) make the selection process more difficult. Unexpectedly, in spite of the small chemical differences between damaged and undamaged hair, the inventors have found that SELEX can be used for the design of aptamers with high binding affinity and specificity for damaged hair, while having reduced binding capacity for undamaged hair.

Selection Library

In SELEX, the initial candidate library is generally a mixture of chemically synthesized DNA oligonucleotides, each comprising a long variable region of n nucleotides flanked, at the 3′ and 5′ ends, by conserved regions or primer recognition regions for all the candidates of the library. These primer recognition regions allow the central variable region to be manipulated during SELEX, in particular by means of PCR.

The length of the variable region determines the diversity of the library, which is equal to 4ⁿ since each position can be occupied by one of four nucleotides A, T, G or C. For long variable regions, huge library complexities arise. For instance, when n=50, the theoretical diversity is 4⁵⁰ or 10³⁰, which is an inaccessible value in practice as it corresponds to more than 10⁵ tons of material for a library wherein each sequence is represented once. The experimental limit is around 10¹⁵ different sequences, which is that of a library wherein all candidates having a variable region of 25 nucleotides are represented. If one chooses to manipulate a library comprising a 30-nucleotide variable region whose theoretical diversity is about 10¹⁸, only 1/1000 of the possibilities will thus be explored. In practice, that is generally sufficient to obtain aptamers having the desired properties. Additionally, since the polymerases used are unreliable and introduce errors at a rate on the order of 10⁻⁴, they contribute to significantly enrich the diversity of the sequence pool throughout the SELEX process: one candidate in 100 will be modified in each amplification cycle for a library with a random region of 100 nucleotides in length, thus leading to the appearance of 10¹³ new candidates for the overall library.

In the present invention, the starting mixture of oligonucleotides may comprise more than about 10⁶ different oligonucleotides and more preferably between about 10¹³ to about 10¹⁵ different oligonucleotides. In the present invention, the length of the variable region may be between about 10 and about 100 nucleotides. In the present invention, the length of the variable region may be between about 20 and about 60 nucleotides. In the present invention, the length of the variable region may be about 40 nucleotides. Random regions shorter than 10 nucleotides may be used, but may be constrained in their ability to form secondary or tertiary structures and in their ability to bind to target molecules. Random regions longer than 100 nucleotides may also be used but may present difficulties in terms of cost of synthesis. The randomness of the variable region is not a constraint of the present invention. For instance, if previous knowledge exists regarding oligonucleotides that bind to a given target, libraries spiked with such sequences may work as well or better than completely random ones.

In the design of primer recognition sequences care should be taken to minimize potential annealing among sequences, fold back regions within sequences, or annealing of the same sequence itself. In the present invention, the length of primer recognition sequences may be between about 10 and about 40 nucleotides. In the present invention, the length of primer recognition sequences may be between about 12 and about 30 nucleotides. In the present invention, the length of primer recognition sequences may be between about 18 and about 26 nucleotides, i.e., about 18, 19, 20, 21, 22, 23, 24, 25 or 26 nucleotides. The length and sequence of the primer recognition sequences determine their annealing temperature. In the present invention, the primer recognition sequences of said oligonucleotides may have an annealing temperature between about 60° C. and about 72° C.

Aptamers can be ribonucleotides (RNA), deoxynucleotides (DNA), or their derivatives. When aptamers are ribonucleotides, the first SELEX step may consist in transcribing the initial mixture of chemically synthesized DNA oligonucleotides via the primer recognition sequence at the 5′ end. After selection, the candidates are converted back into DNA by reverse transcription before being amplified. RNA and DNA aptamers having comparable characteristics have been selected against the same target and reported in the art. Additionally, both types of aptamers can be competitive inhibitors of one another, suggesting potential overlapping of interaction sites.

New functionalities, such as hydrophobicity or photoreactivity, can be incorporated into the oligonucleotides by modifications of the nucleobases before or after selection. Modifications at the C-5 position of pyrimidines or at the C-8 or N-7 positions of purines are especially common and compatible with certain enzymes used during the amplification step in SELEX. In the present invention, said oligonucleotides may comprise natural or non-natural nucleobases. Natural nucleobases are adenine, cytosine, guanine, thymine, and uracil. Non-limiting examples of non-natural nucleobases are hypoxanthine, xanthine, 7-methylguanine, 5,6-dihydrouracil, 5-5-methylcytosine, 5-hydroxymethylcytosine, thiouracil, 1-methylhypoxanthine, 6-methylisoquinoline-1-thione-2-yl, 3-methoxy-2-naphthyl, 5-propynyluracil-1-yl, 5-methylcytosin-1-yl, 2-aminoadenin-9-yl, 7-deaza-7-iodoadenin-9-yl, 7-deaza-7-propynyl-2-aminoadenin-9-yl, phenoxazinyl, phenoxazinyl-G-clam, 5-bromouracil, 5-iodouracil, and mixtures thereof. Some non-natural nucleobases, such as 5-bromouracil or 5-iodouracil, can be used to generate photo-cross-linkable aptamers, which can be activated by UV light to form a covalent link with the target.

In the present invention, the nucleosides of said oligonucleotides may be linked by a chemical motif selected from the group comprising: natural phosphate diester, chiral phosphorothionate, chiral methyl phosphonate, chiral phosphoramidate, chiral phosphate chiral triester, chiral boranophosphate, chiral phosphoroselenoate, phosphorodithioate, phosphorothionate amidate, methylenemethylimino, 3′-amide, 3′ achiral phosphoramidate, 3′ achiral methylene phosphonates, thioformacetal, thioethyl ether, fluorophosphate, and mixtures thereof. In the present invention, the nucleosides of said oligonucleotides may be linked by natural phosphate diesters.

In the present invention, the sugar moiety of the nucleosides of said oligonucleotides may be selected from the group comprising: ribose, deoxyribose, 2′-fluoro deoxyribose, 2′-O-methyl ribose, 2′-O-(3-amino)propyl ribose, 2′-O-(2-methoxy)ethyl ribose, 2′-O-2-(N,N-dimethylaminooxy)ethyl ribose, 2′-O-2-[2-(N,N-dimethylamino)ethyloxy]ethyl ribose, 2′-O-N,N-dimethylacetamidyl ribose, N-morpholinophosphordiamidate, α-deoxyribofuranosyl, other pentoses, hexoses, and mixtures thereof.

In the present invention, said derivatives of ribonucleotides or said derivatives of deoxyribonucleotides may be selected from the group comprising: locked oligonucleotides, peptide oligonucleotides, glycol oligonucleotides, threose oligonucleotides, hexitol oligonucleotides, altritol oligonucleotides, butyl oligonucleotides, L-ribonucleotides, arabino oligonucleotides, 2′-fluoroarabino oligonucleotides, cyclohexene oligonucleotides, phosphorodiamidate morpholino oligonucleotides, and mixtures thereof.

When using modified nucleotides during the SELEX process, they should be compatible with the enzymes used during the amplification step. Non-limiting examples of modifications that are compatible with commercial enzymes include modifications at the 2′ position of the sugar in RNA libraries. The ribose 2′—OH group of pyrimidine nucleotides can be replaced with 2′-amino, 2′-fluoro, 2′-methyl, or 2′-O-methyl, which protect the RNA from degradation by nucleases. Additional modifications in the phosphate linker, such as phosphorothionate and boranophosphate, are also compatible with the polymerases and confer resistance to nucleases.

In the present invention, at least one nucleotide of said oligonucleotides may be fluorinated at the 2′ position of the pentose group. In the present invention, the pyrimidine nucleotides of said oligonucleotides may be at least partially fluorinated at the 2′ position of the pentose group. In the present invention, all the pyrimidine nucleotides of said oligonucleotides may be fluorinated at the 2′ position of the pentose group. In the present invention, at least one nucleotide of said oligonucleotides may be aminated at the 2′ position of the pentose group.

Another approach, recently described as two-dimensional SELEX, simultaneously applies in vitro oligonucleotide selection and dynamic combinatorial chemistry (DCC), e.g., a reversible reaction between certain groups of the oligonucleotide (amine groups) and a library of aldehyde compounds. The reaction produces imine oligonucleotides which are selected on the same principles as for conventional SELEX. It is thus possible to identify for a target hairpin RNA modified aptamers that differ from natural aptamers.

A very different approach relates to the use of optical isomers. Natural oligonucleotides are D-isomers. L-analogs are resistant to nucleases but cannot be synthesized by polymerases. According to the laws of optical isomerism, an L-series aptamer can form with its target (T) a complex having the same characteristics as the complex formed by the D-series isomer and the enantiomer (T′) of the target (T). Consequently, if compound T′ can be chemically synthesized, it can be used to perform the selection of a natural aptamer (D). Once identified, this aptamer can be chemically synthesized in an L-series. This L-aptamer is a ligand of the natural target (T).

Selection Step

Single stranded oligonucleotides can fold to generate secondary and tertiary structures, resembling the formation of base pairs. The initial sequence library is thus a library of three-dimensional shapes, each corresponding to a distribution of units that can trigger electrostatic interactions, create hydrogen bonds, etc. Selection becomes a question of identifying in the library the shape suited to the target, i.e., the shape allowing the greatest number of interactions and the formation of the most stable aptamer-target complex. For small targets (dyes, antibiotics, etc.) the aptamers identified are characterized by equilibrium dissociation constants in the micromolar range, whereas for protein targets K_{d} values below 10⁻⁹ M are not rare.

Selection in each round occurs by means of physical separation of oligonucleotides associated with the target from free oligonucleotides. Multiple techniques may be applied (chromatography, filter retention, electrophoresis, etc.). The selection conditions are adjusted (relative concentration of target/candidates, ion concentration, temperature, washing, etc.) so that a target-binding competition occurs between the oligonucleotides. Generally, stringency is increased as the rounds proceed in order to promote the capture of oligonucleotides with the highest affinity. In addition, counter-selections or negative selections are carried out to eliminate oligonucleotides that recognize the support or unwanted targets (e.g., filter, beads, etc.).

The SELEX process for the selection of target-specific aptamers is characterized by repetition of five main steps: binding of oligonucleotides to the target, partition or removal of oligonucleotides with low binding affinity, elution of oligonucleotides with high binding affinity, amplification or replication of oligonucleotides with high binding affinity, and conditioning or preparation of the oligonucleotides for the next cycle. This selection process is designed to identify the oligonucleotides with the greatest affinity and specificity for the target material.

In the present invention, a method of designing an aptamer composition may comprise the step of contacting: a) a mixture of oligonucleotides, b) a selection buffer, and c) a target material selected from the group consisting of: undamaged hair, damaged hair, hair cuticle, hair epicuticle, hair exocuticle, hair endocuticle, hair cortex, hair keratins, hair F-layer, hair lipids, 18-methyleicosanoic acid, and mixtures thereof. In the present inveniton, said target material may be selected from the group consisting of: undamaged hair, damaged hair, and mixtures thereof. In the present invention said target material may be damaged hair. In the present invention, said mixture of oligonucleotides comprises oligonucleotides may be selected from the group consisting of deoxyribonucleotides, ribonucleotides, derivatives of deoxyribonucleotides, derivatives of ribonucleotides, and mixtures thereof.

SELEX cycles are usually repeated several times until oligonucleotides with high binding affinity are identified. The number of cycles depends on multiple variables, including target features and concentration, design of the starting random oligonucleotide library, selection conditions, ratio of target binding sites to oligonucleotides, and the efficiency of the partitioning step. In the present invention, said contacting step may be performed at least 5 times. In the present invention, said contacting step may be performed between 6 and 15 times. In the present invention, said method may further comprise the step of removing the oligonucleotides that do not bind said target material during said contacting step.

Oligonucleotides are oligo-anions, each unit having a charge and hydrogen-bond donor/acceptor sites at a particular pH. Thus, the pH and ionic strength of the selection buffer are important and should represent the conditions of the intended aptamer application. In the present invention, the pH of said selection buffer may be between about 2 and about 9. In the present invention, the pH of said selection buffer may be between about 5 and about 8.

Cations can not only facilitate the proper folding of the oligonucleotides, but also can provide benefits to the hair or the scalp. In the present invention, said selection buffer may comprise cations. Non-limiting examples of cations are Mg²⁺, Ca²⁺, Sn²⁺, Sn⁴⁺, Zn²⁺, A1³⁺, Cu²⁺, Fe²⁺, and Fe³⁺.

In order for the aptamers to maintain their structures and function during their application, the in vitro selection process can be carried out under conditions similar to those for which they are being developed. In the present invention, said selection buffer may comprise a solution or suspension of a hair care composition selected from the group comprising shampoos, conditioning shampoos, pet shampoo, leave-in treatments, sprays, liquids, pastes, Newtonian or non-Newtonian fluids, gels, and sols. In the present invention, said selection buffer may comprise a solution of a shampoo.

In the present invention, said selection buffer may comprise at least one surfactant. In the present invention, said at least one surfactant may be selected from the group consisting of anionic surfactants, amphoteric or zwitterionic surfactants, and mixtures thereof. Non-limiting examples of anionic surfactants are alkyl and alkyl ether sulfates or sulfonates, including ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, sodium cocoyl isethionate and combinations thereof. Non-limiting amphoteric surfactants include those surfactants broadly described as derivatives of aliphatic secondary and tertiary amines in which the aliphatic radical can be straight or branched chain and wherein one of the aliphatic substituents contains from about 8 to about 18 carbon atoms and one contains an anionic group such as carboxy, sulfonate, sulfate, phosphate, or phosphonate, including cocoamphoacetate, cocoamphodiacetate, lauroamphoacetate, lauroamphodiacetate, and mixtures thereof. Non-limiting examples of zwitterionic surfactants include those surfactants broadly described as derivatives of aliphatic quaternaryammonium, phosphonium, and sulfonium compounds, in which the aliphatic radicals can be straight or branched chain, and wherein one of the aliphatic substituents contains from about 8 to about 18 carbon atoms and one contains an anionic group such as carboxy, sulfonate, sulfate, phosphate or phosphonate, and betaines.

In the present invention, said selection buffer may comprise at least one material selected from the group compressing: aqueous carriers, gel matrixes, silicone conditioning agents, organic conditioning materials, non-ionic polymers, deposition aids, rheology modifier/suspending agents, benefit agents, and mixtures thereof. Non-limiting examples of aqueous carriers are water and water solutions of lower alkyl alcohols and polyhydric alcohols, including ethanol, isopropanol, propylene glycol, hexylene glycol, glycerin, and propane diol. Non-limiting examples of gel matrixes include water solutions of fatty alcohols, including cetyl alcohol, stearyl alcohol, behenyl alcohol, and mixtures thereof. Non-limiting examples of silicone conditioning agents include dimethicones, dimethiconols, cyclic silicones, methylphenyl polysiloxane, and modified silicones with various functional groups such as amino groups, quaternary ammonium salt groups, aliphatic groups, alcohol groups, carboxylic acid groups, ether groups, sugar or polysaccharide groups, fluorine-modified alkyl groups, alkoxy groups, or combinations of such groups. Non-limiting examples of organic conditioning materials include hydrocarbon oils, polyolefins, fatty esters, fluorinated conditioning compounds, fatty alcohols, alkyl glucosides and alkyl glucoside derivatives, quaternary ammonium compounds, polyethylene glycols and polypropylene glycols having a molecular weight of up to about 2,000,000 including those with CTFA names PEG-200, PEG-400, PEG-600, PEG-1000, PEG-2M, PEG-7M, PEG-14M, PEG-45M and mixtures thereof. Non-limiting examples of non-ionic polymers include polyalkylene glycols, such as polyethylene glycols. Non-limiting examples of deposition aids include copolymers of vinyl monomers having cationic amine or quaternary ammonium functionalities with water soluble spacer monomers such as acrylamide, methacrylamide, alkyl and dialkyl acrylamides, alkyl and dialkyl methacrylamides, alkyl acrylate, alkyl methacrylate, vinyl caprolactone, and vinyl pyrrolidone; vinyl esters, vinyl alcohol (made by hydrolysis of polyvinyl acetate), maleic anhydride, propylene glycol, and ethylene glycol, cationic celluloses, cationic starches, and cationic guar gums. Non-limiting examples of rheology modifier/suspending agents include homopolymers based on acrylic acid, methacrylic acid or other related derivatives; alginic acid-based materials; and cellulose derivatives. Non-limiting examples of benefit agents include brightening agents, strengthening agents, anti-fungal agents, anti-bacterial agents, anti-microbial agents, anti-dandruff agents, anti-malodor agents, perfumes, olfactory enhancement agents, anti-itch agents, cooling agents, anti-adherence agents, moisturization agents, smoothness agents, surface modification agents, antioxidants, natural extracts and essential oils, dyes, pigments, bleaches, nutrients, peptides, vitamins, enzymes, chelants, and mixtures thereof.

Negative selection or counter-selection steps can minimize the enrichment of oligonucleotides that bind to undesired targets or undesired epitopes within a target. For hair care applications, preferential binding of aptamers to damaged hair versus undamaged hair may be desirable. In the present invention, said method of designing an aptamer composition may further comprise the step of contacting: a) a mixture of oligonucleotides, b) a selection buffer, and c) undamaged hair. Methods for negative selection or counter-selection of aptamers against unbound targets have been published in WO201735666, the content of which is incorporated herein by reference.

In the present invention, the method of designing an aptamer composition may comprise the steps of: a) synthesizing a mixture of oligonucleotides; b) contacting: i. said mixture of oligonucleotides, ii. a selection buffer, and iii. a target material selected from the group consisting of: undamaged hair, damaged hair, hair cuticle, hair epicuticle, hair exocuticle, hair endocuticle, hair cortex, hair keratins, hair F-layer, hair lipids, 18-methyleicosanoic acid, and mixtures thereof, to produce a target suspension; c) removing the liquid phase from said target suspension to produce a target-oligonucleotide mixture; d) contacting said target-oligonucleotide mixture with a washing buffer and removing the liquid phase to produce a target-aptamer mixture; and e) contacting said target-aptamer mixture with an elution buffer and recovering the liquid phase to produce an aptamer mixture. In the present invention, said steps may be performed repetitively at least 5 times. In the present invention, said steps may be performed between 6 and 15 times.

In the present invention, the method of designing an aptamer composition may comprise the steps of: a) synthesizing a random mixture of deoxyribonucleotides comprising oligonucleotides consisting of: i. a T7 promoter sequence at the 5′-end, ii. a variable 40-nucleotide sequence in the middle, and iii. a conserved reverse primer recognition sequence at the 3′ end; b) contacting: i. said random mixture of deoxyribonucleotides, ii. a selection buffer, and iii. a sample of hair, to produce a target suspension; c) removing the liquid phase from said target suspension to produce a hair-oligonucleotide mixture; d) contacting said hair-oligonucleotide mixture with a washing buffer and removing the liquid phase to produce a hair-aptamer mixture; e) contacting said hair-aptamer mixture with an elution buffer and recovering the liquid phase to produce a DNA aptamer mixture; f) amplifying said DNA aptamer mixture to produce an enriched mixture of deoxyribonucleotides; and g) sequencing said enriched mixture of deoxyribonucleotides.

Post-Selection Modification

To enhance stability of the aptamers, chemical modifications can be introduced in the aptamer after the selection process. For instance, the 2′-OH groups of the ribose moieties can be replaced by 2′-fluoro, 2′-amino, or 2′-O-methyl groups. Furthermore, the 3′- and 5′-ends of the aptamers can be capped with different groups, such as streptavidin-biotin, inverted thymidine, amine, phosphate, polyethylene-glycol, cholesterol, fatty acids, proteins, enzymes, fluorophores, among others, making the oligonucleotides resistant to exonucleases or providing some additional benefits. Other modifications are described in previous sections of the present disclosure.

Unlike backbone modifications which can cause aptamer-target interaction properties to be lost, it is possible to conjugate various groups at one of the 3′- or 5′-ends of the oligonucleotide in order to convert it into a delivery vehicle, tool, probe, or sensor without disrupting its characteristics. This versatility constitutes a significant advantage of aptamers, in particular for their application in the current invention. In the present invention, one or more hair care active ingredients may be covalently attached to the 3′-end of said at least one oligonucleotide. In the present invention, one or more hair care active ingredients may be covalently attached to the 5′-end of said at least one oligonucleotide. In the present invention, one or more hair care active ingredients may be covalently attached to random positions of said at least one oligonucleotide.

Incorporation of modifications to aptamers can be performed using enzymatic or chemical methods. Non-limiting examples of enzymes used for modification of aptamers are terminal deoxynucleotidyl transferases (TdT), T4 RNA ligases, T4 polynucleotide kinases (PNK), DNA polymerases, RNA polymerases, and other enzymes known by those skilled in the art. TdTs are template-independent polymerases that can add modified deoxynucleotides to the 3′ terminus of deoxyribonucleotides. T4 RNA ligases can be used to label ribonucleotides at the 3′-end by using appropriately modified nucleoside 3′,5′-bisphosphates. PNK can be used to phosphorylate the 5′-end of synthetic oligonucleotides, enabling other chemical transformations (see below). DNA and RNA polymerases are commonly used for the random incorporation of modified nucleotides throughout the sequence, provided such nucleotides are compatible with the enzymes.

Non-limiting examples of chemical methods used for modification of aptamers are periodate oxidation of ribonucleotides, EDC activation of 5′-phosphate, random chemical labeling methods, and other chemical methods known by those skilled in the art, incorporated herein as for the current invention.

During periodate oxidation, meta- and ortho-perdionates cleave the C—C bonds between vicinal diols of 3′-ribonucleotides, creating two aldehyde moieties that enable the conjugation of labels or active ingredients at the 3′-end of RNA aptamers. The resulting aldehydes can be easily reacted with hydrazide- or primary amine-containing molecules. When amines are used, the produced Schiff bases can be reduced to more stable secondary amines with sodium cyanoborohydride (NaBH₄).

When EDC activation of 5′-phosphate is used, the 5′-phosphate of oligonucleotides is frequently activated with EDC (1-Ethyl-3-[3-dimethylaminopropyl]carbodiimide hydrochloride) and imidazole to produce a reactive imidazolide intermediate, followed by reaction with a primary amine to generate aptamers modified at the 5′ end. Because the 5′ phosphate group is required for the reaction, synthetic oligonucleotides can be first treated with a kinase (e.g. PNK).

Random chemical labeling can be performed with different methods. Because they allow labeling at random sites along the aptamer, a higher degree of modification can be achieved compared to end-labeling methods. However, since the nucleobases are modified, binding of the aptamers to their target can be disrupted. The most common random chemical modification methods involve the use of photoreactive reagents, such as phenylazide-based reagents. When the phenylazide group is exposed to UV light, it forms a labile nitrene that reacts with double bonds and C—H and N—H sites of the aptamers.

Additional information about methods for modification of aptamers is summarized in “Hermanson G. T. (2008). Bioconjugate Techniques. 2nd Edition. pp. 969-1002, Academic Press, San Diego.”, the content of which is incorporated herein by reference.

After selection, in addition to chemical modifications, sequence truncations can be performed to remove regions that are not essential for binding or for folding into the structure. Moreover, aptamers can be linked together to provide different features or better affinity. Thus, any truncations or combinations of the aptamers described herein are incorporated as part of the current invention.

### IV. Application of Aptamer Compositions in Hair Care Products

The aptamers of the current invention can be used in hair care compositions to provide one or more benefits.

### Shampoo Composition

The hair care composition of the present invention can be a shampoo. The shampoo composition comprises from about 0.001% to about 1%, alternatively from about 0.01% to about 0.5%, alternatively from about 0.1% to about 0.3% of one or more aptamer.

A. Detersive Surfactant

The shampoo composition may comprise one or more detersive surfactants, which provides cleaning performance to the composition. The one or more detersive surfactants in turn may comprise an anionic surfactant, amphoteric or zwitterionic surfactants, or mixtures thereof. Various examples and descriptions of detersive surfactants are set forth in U.S. Pat. No. 6,649,155; U.S. Patent Application Publication No. 2008/0317698; and U.S. Patent Application Publication No. 2008/0206355, which are incorporated herein by reference in their entirety.

The concentration of the detersive surfactant component in the shampoo composition should be sufficient to provide the desired cleaning and lather performance, and generally ranges from about 2 wt % to about 50 wt %, from about 5 wt % to about 30 wt %, from about 8 wt % to about 25 wt %, from about 10 wt % to about 20 wt %, about 5 wt %, about 10 wt %, about 12 wt %, about 15 wt %, about 17 wt %, about 18 wt %, or about 20 wt %.

Anionic surfactants suitable for use in the compositions are the alkyl and alkyl ether sulfates. Other suitable anionic surfactants are the water-soluble salts of organic, sulfuric acid reaction products. Still other suitable anionic surfactants are the reaction products of fatty acids esterified with isethionic acid and neutralized with sodium hydroxide. Other similar anionic surfactants are described in U.S. Pat. Nos. 2,486,921; 2,486,922; and 2,396,278, which are incorporated herein by reference in their entirety.

Exemplary anionic surfactants for use in the shampoo composition include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, sodium cocoyl isethionate and combinations thereof. In the present invention, the anionic surfactant may be sodium lauryl sulfate or sodium laureth sulfate.

Suitable amphoteric or zwitterionic surfactants for use in the shampoo composition herein include those which are known for use in shampoo or other personal care cleansing. Concentrations of such amphoteric surfactants range from about 0.5 wt % to about 20 wt %, and from about 1 wt % to about 10 wt %. Non limiting examples of suitable zwitterionic or amphoteric surfactants are described in U.S. Pat. Nos. 5,104,646 and 5,106,609, which are incorporated herein by reference in their entirety.

Amphoteric detersive surfactants suitable for use in the shampoo composition include those surfactants broadly described as derivatives of aliphatic secondary and tertiary amines in which the aliphatic radical can be straight or branched chain and wherein one of the aliphatic substituents contains from about 8 to about 18 carbon atoms and one contains an anionic group such as carboxy, sulfonate, sulfate, phosphate, or phosphonate. Exemplary amphoteric detersive surfactants for use in the present shampoo composition include cocoamphoacetate, cocoamphodiacetate, lauroamphoacetate, lauroamphodiacetate, and mixtures thereof.

Zwitterionic detersive surfactants suitable for use in the shampoo composition include those surfactants broadly described as derivatives of aliphatic quaternaryammonium, phosphonium, and sulfonium compounds, in which the aliphatic radicals can be straight or branched chain, and wherein one of the aliphatic substituents contains from about 8 to about 18 carbon atoms and one contains an anionic group such as carboxy, sulfonate, sulfate, phosphate or phosphonate. In the present invention, zwitterionics such as betaines may be selected.

Non limiting examples of other anionic, zwitterionic, amphoteric or optional additional surfactants suitable for use in the shampoo composition are described in McCutcheon's, Emulsifiers and Detergents, 1989 Annual, published by M. C. Publishing Co., and U.S. Pat. Nos. 3,929,678, 2,658,072; 2,438,091; 2,528,378, which are incorporated herein by reference in their entirety.

The shampoo composition may also comprise a shampoo gel matrix, an aqueous carrier, and other additional ingredients described herein.

B. Aqueous Carrier

The shampoo composition comprises an aqueous carrier. Accordingly, the formulations of the shampoo composition can be in the form of pourable liquids (under ambient conditions). Such compositions will therefore typically comprise an aqueous carrier, which is present at a level of at least 20 wt %, from about 20 wt % to about 95 wt %, or from about 60 wt % to about 85 wt %. The aqueous carrier may comprise water, or a miscible mixture of water and organic solvent, and in one aspect may comprise water with minimal or no significant concentrations of organic solvent, except as otherwise incidentally incorporated into the composition as minor ingredients of other components.

The aqueous carriers useful in the shampoo composition include water and water solutions of lower alkyl alcohols and polyhydric alcohols. The lower alkyl alcohols useful herein are monohydric alcohols having 1 to 6 carbons, in one aspect, ethanol and isopropanol. The polyhydric alcohols useful herein include propylene glycol, hexylene glycol, glycerin, and propane diol.

### Conditioner Composition

The hair care composition of the present invention can be a hair conditioner. The hair conditioner composition described herein comprises (i) from about 0.001% to about 1%, alternatively from about 0.01% to about 0.5%, alternatively from about 0.1% to about 0.3% of one or more aptamer. The conditioner composition may also comprise a conditioner gel matrix comprising (1) one or more high melting point fatty compounds, (2) a cationic surfactant system, and (3) a second aqueous carrier.

A. Cationic Surfactant System

The conditioner gel matrix of the conditioner composition includes a cationic surfactant system. The cationic surfactant system can be one cationic surfactant or a mixture of two or more cationic surfactants. The cationic surfactant system can be selected from: mono-long alkyl quaternized ammonium salt; a combination of mono-long alkyl quaternized ammonium salt and di-long alkyl quaternized ammonium salt; mono-long alkyl amidoamine salt; a combination of mono-long alkyl amidoamine salt and di-long alkyl quaternized ammonium salt, a combination of mono-long alkyl amindoamine salt and mono-long alkyl quaternized ammonium salt.

The cationic surfactant system can be included in the composition at a level by weight of from about 0.1% to about 10%, from about 0.5% to about 8%, from about 0.8% to about 5%, and from about 1.0% to about 4%.

Mono-Long Alkyl Quaternized Ammonium Salt

The monoalkyl quaternized ammonium salt cationic surfactants useful herein are those having one long alkyl chain which has about 22 carbon atoms and in may be a C22 alkyl group. The remaining groups attached to nitrogen are independently selected from an alkyl group of from 1 to about 4 carbon atoms or an alkoxy, polyoxyalkylene, alkylamido, hydroxyalkyl, aryl or alkylaryl group having up to about 4 carbon atoms.

Mono-long alkyl quaternized ammonium salts useful herein are those having the formula (I):

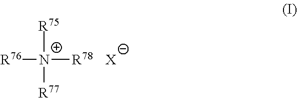

wherein one of R⁷⁵, R⁷⁶, R⁷⁷ and R⁷⁸ is selected from an alkyl group of 22 carbon atoms or an aromatic, alkoxy, polyoxyalkylene, alkylamido, hydroxyalkyl, aryl or alkylaryl group having up to about 30 carbon atoms; the remainder of R⁷⁵, R⁷⁶, R⁷⁷ and R⁷⁸ are independently selected from an alkyl group of from 1 to about 4 carbon atoms or an alkoxy, polyoxyalkylene, alkylamido, hydroxyalkyl, aryl or alkylaryl group having up to about 4 carbon atoms; and X⁻ is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The alkyl groups can contain, in addition to carbon and hydrogen atoms, ether and/or ester linkages, and other groups such as amino groups. The longer chain alkyl groups, e.g., those of about 22 carbons, or higher, can be saturated or unsaturated. One of R⁷⁵, R⁷⁶, R⁷⁷ and R⁷⁸ can be selected from an alkyl group of about 22 carbon atoms, the remainder of R⁷⁵, R⁷⁶, R⁷⁷ and R⁷⁸ are independently selected from CH₃, C₂H₅, C₂H₄OH, and mixtures thereof; and X is selected from the group consisting of Cl, Br, CH₃OSO₃, C₂H₅OSO₃, and mixtures thereof.

Nonlimiting examples of such mono-long alkyl quaternized ammonium salt cationic surfactants include: behenyl trimethyl ammonium salt.

Mono-Long Alkyl Amidoamine Salt

Mono-long alkyl amines are also suitable as cationic surfactants. Primary, secondary, and tertiary fatty amines are useful. Particularly useful are tertiary amido amines having an alkyl group of about 22 carbons. Exemplary tertiary amido amines include: behenamidopropyldimethylamine, behenamidopropyldiethylamine, behenamidoethyldiethylamine, behenamidoethyldimethylamin. Useful amines in the present invention are disclosed in U.S. Pat. No. 4,275,055, Nachtigal, et al. These amines can also be used in combination with acids such as l-glutamic acid, lactic acid, hydrochloric acid, malic acid, succinic acid, acetic acid, fumaric acid, tartaric acid, citric acid, l-glutamic hydrochloride, maleic acid, and mixtures thereof; and may be l-glutamic acid, lactic acid, and/or citric acid. The amines herein can be partially neutralized with any of the acids at a molar ratio of the amine to the acid of from about 1:0.3 to about 1:2, and/or from about 1:0.4 to about 1:1.

Di-Long Alkyl Quaternized Ammonium Salt

Di-long alkyl quaternized ammonium salt can be combined with a mono-long alkyl quaternized ammonium salt or mono-long alkyl amidoamine salt. It is believed that such combination can provide easy-to rinse feel, compared to single use of a monoalkyl quaternized ammonium salt or mono-long alkyl amidoamine salt. In such combination with a mono-long alkyl quaternized ammonium salt or mono-long alkyl amidoamine salt, the di-long alkyl quaternized ammonium salts are used at a level such that the wt % of the dialkyl quaternized ammonium salt in the cationic surfactant system is in the range of from about 10% to about 50%, and/or from about 30% to about 45%.

The di-long alkyl quaternized ammonium salt cationic surfactants useful herein are those having two long alkyl chains having about 22 carbon atoms. The remaining groups attached to nitrogen are independently selected from an alkyl group of from 1 to about 4 carbon atoms or an alkoxy, polyoxyalkylene, alkylamido, hydroxyalkyl, aryl or alkylaryl group having up to about 4 carbon atoms.

Di-long alkyl quaternized ammonium salts useful herein are those having the formula (II):

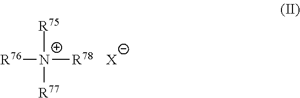

wherein two of R⁷⁵, R⁷⁶, R⁷⁷ and R⁷⁸ is selected from an alkyl group of from 22 carbon atoms or an aromatic, alkoxy, polyoxyalkylene, alkylamido, hydroxyalkyl, aryl or alkylaryl group having up to about 30 carbon atoms; the remainder of R⁷⁵, R⁷⁶, R⁷⁷ and R⁷⁸ are independently selected from an alkyl group of from 1 to about 4 carbon atoms or an alkoxy, polyoxyalkylene, alkylamido, hydroxyalkyl, aryl or alkylaryl group having up to about 4 carbon atoms; and X⁻ is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The alkyl groups can contain, in addition to carbon and hydrogen atoms, ether and/or ester linkages, and other groups such as amino groups. The longer chain alkyl groups, e.g., those of about 22 carbons, or higher, can be saturated or unsaturated. One of R⁷⁵, R⁷⁶, R⁷⁷ and R⁷⁸ can be selected from an alkyl group of from 22 carbon atoms, the remainder of R⁷⁵, R⁷⁶, R⁷⁷ and R⁷⁸ are independently selected from CH₃, C₂H₅, C₂H₄OH, and mixtures thereof; and X is selected from the group consisting of Cl, Br, CH₃OSO₃, C₂H₅OSO₃, and mixtures thereof.

Such dialkyl quaternized ammonium salt cationic surfactants include, for example, dialkyl (C22) dimethyl ammonium chloride, ditallow alkyl dimethyl ammonium chloride, dihydrogenated tallow alkyl dimethyl ammonium chloride. Such dialkyl quaternized ammonium salt cationic surfactants also include, for example, asymmetric dialkyl quaternized ammonium salt cationic surfactants.

B. High Melting Point Fatty Compound

The conditioner gel matrix of the conditioner composition includes one or more high melting point fatty compounds. The high melting point fatty compounds useful herein may have a melting point of 25° C. or higher, and is selected from the group consisting of fatty alcohols, fatty acids, fatty alcohol derivatives, fatty acid derivatives, and mixtures thereof. It is understood by the artisan that the compounds disclosed in this section of the specification can in some instances fall into more than one classification, e.g., some fatty alcohol derivatives can also be classified as fatty acid derivatives. However, a given classification is not intended to be a limitation on that particular compound, but is done so for convenience of classification and nomenclature. Further, it is understood by the artisan that, depending on the number and position of double bonds, and length and position of the branches, certain compounds having certain carbon atoms may have a melting point of less than 25° C. Such compounds of low melting point are not intended to be included in this section. Nonlimiting examples of the high melting point compounds are found in International Cosmetic Ingredient Dictionary, Fifth Edition, 1993, and CTFA Cosmetic Ingredient Handbook, Second Edition, 1992.

Among a variety of high melting point fatty compounds, fatty alcohols are suitable for use in the conditioner composition. The fatty alcohols useful herein are those having from about 14 to about 30 carbon atoms, from about 16 to about 22 carbon atoms. These fatty alcohols are saturated and can be straight or branched chain alcohols. Suitable fatty alcohols include, for example, cetyl alcohol, stearyl alcohol, behenyl alcohol, and mixtures thereof.

High melting point fatty compounds of a single compound of high purity can be used. Single compounds of pure fatty alcohols selected from the group of pure cetyl alcohol, stearyl alcohol, and behenyl alcohol can also be used. By “pure” herein, what is meant is that the compound has a purity of at least about 90%, and/or at least about 95%. These single compounds of high purity provide good rinsability from the hair when the consumer rinses off the composition.

The high melting point fatty compound can be included in the conditioner composition at a level of from about 0.1% to about 20%, alternatively from about 1% to about 15%, and alternatively from about 1.5% to about 8% by weight of the composition, in view of providing improved conditioning benefits such as slippery feel during the application to wet hair, softness and moisturized feel on dry hair.

C. Aqueous Carrier

The conditioner gel matrix of the conditioner composition includes an aqueous carrier. Accordingly, the formulations of the conditioner composition can be in the form of pourable liquids (under ambient conditions). Such compositions will therefore typically comprise a second aqueous carrier, which is present at a level of from about 20 wt % to about 95 wt %, or from about 60 wt % to about 85 wt %. The aqueous carrier may comprise water, or a miscible mixture of water and organic solvent, and in one aspect may comprise water with minimal or no significant concentrations of organic solvent, except as otherwise incidentally incorporated into the composition as minor ingredients of other components.

The aqueous carriers useful in the conditioner composition include water and water solutions of lower alkyl alcohols and polyhydric alcohols. The lower alkyl alcohols useful herein are monohydric alcohols having 1 to 6 carbons, in one aspect, ethanol and isopropanol. The polyhydric alcohols useful herein include propylene glycol, hexylene glycol, glycerin, and propane diol.

### Leave-on Treatment

The hair care composition of the present invention can be leave-on treatment. The leave-on treatment composition described herein may comprise from about 0.001% to about 1%, alternatively from about 0.01% to about 0.5%, alternatively from about 0.1% to about 0.3% of one or more aptamer. The leave-on treatment may also comprises (1) one or more rheology modifiers and (2) an aqueous carrier.

A. Rheology Modifier

The leave-on treatment may include one or more rheology modifiers to adjust the rheological characteristics of the composition for better feel, in-use properties and the suspending stability of the composition. For example, the rheological properties are adjusted so that the composition remains uniform during its storage and transportation and it does not drip undesirably onto other areas of the body, clothing or home furnishings during its use. Any suitable rheology modifier can be used. In the present invention, the leave-on treatment may comprise from about 0.01% to about 3% of a rheology modifier, alternatively from about 0.1% to about 1% of a rheology modifier,

The one or more rheology modifier may be selected from the group consisting of polyacrylamide thickeners, cationically modified polysaccharides, associative thickeners, and mixtures thereof. Associative thickeners include a variety of material classes such as, for example: hydrophobically modified cellulose derivatives; hydrophobically modified alkoxylated urethane polymers, such as PEG-150/decyl alcohol/SMDI copolymer, PEG-150/stearyl alcohol/SMDI copolymer, polyurethane-39; hydrophobically modified, alkali swellable emulsions, such as hydrophobically modified polypolyacrylates, hydrophobically modified polyacrylic acids, and hydrophobically modified polyacrylamides; hydrophobically modified polyethers. These materials may have a hydrophobe that can be selected from cetyl, stearyl, oleayl, and combinations thereof, and a hydrophilic portion of repeating ethylene oxide groups with repeat units from 10-300, alternatively from 30-200, and alternatively from 40-150. Examples of this class include PEG-120-methylglucose dioleate, PEG-(40 or 60) sorbitan tetraoleate, PEG-150 pentaerythrityl tetrastearate, PEG-55 propylene glycol oleate, PEG-150 distearate.

Non-limiting examples of additional rheology modifiers include acrylamide/ammonium acrylate copolymer (and)polyisobutene (and) polysorbate 20; acrylamide/sodium acryloyldimethyl taurate copolymer/isohexadecane/polysorbate 80; acrylates copolymer; acrylates/beheneth-25 methacrylate copolymer; acrylates/C10-C30 alkyl acrylate crosspolymer; acrylates/steareth-20 itaconate copolymer; ammonium polyacrylate/Isohexadecane/PEG-40 castor oil; C12-16 alkyl PEG-hydroxypropylhydroxyethyl ethylcellulose (HM-EHEC); carbomer; crosslinked polyvinylpyrrolidone (PVP); dibenzylidene sorbitol; hydroxyethyl ethylcellulose (EHEC); hydroxypropyl methylcellulose (HPMC); hydroxypropyl methylcellulose (HPMC); hydroxypropylcellulose (HPC); methylcellulose (MC); methylhydroxyethyl cellulose (MEHEC); PEG-150/decyl alcohol/SMDI copolymer; PEG-150/stearyl alcohol/SMDI copolymer; polyacrylamide/C13-14 isoparaffin/laureth-7; polyacrylate 13/polyisobutene/polysorbate 20; polyacrylate crosspolymer-6; polyamide-3; polyquaternium-37 (and) hydrogenated polydecene (and) trideceth-6; polyurethane-39; sodium acrylate/acryloyldimethyltaurate/dimethylacrylamide; crosspolymer (and) isohexadecane (and) polysorbate 60; sodium polyacrylate. Exemplary commercially-available rheology modifiers include ACULYN™ 28, Klucel M CS, Klucel H CS, Klucel G CS, SYLVACLEAR AF1900V, SYLVACLEAR PA1200V, Benecel E10M, Benecel K35M, Optasense RMC70, ACULYN™33, ACULYN™46, ACULYN™22, ACULYN™44, Carbopol Ultrez 20, Carbopol Ultrez 21, Carbopol Ultrez 10, Carbopol 1342, Sepigel™ 305, Simulgel™600, Sepimax Zen, and/or combinations thereof.

B. Aqueous Carrier

The leave-on treatment may comprise an aqueous carrier. Accordingly, the formulations of the leave-on treatment can be in the form of pourable liquids (under ambient conditions). Such compositions will therefore typically comprise an aqueous carrier, which is present at a level of at least 20 wt %, from about 20 wt % to about 95 wt %, or from about 60 wt % to about 85 wt %. The aqueous carrier may comprise water, or a miscible mixture of water and organic solvent, and in one aspect may comprise water with minimal or no significant concentrations of organic solvent, except as otherwise incidentally incorporated into the composition as minor ingredients of other components.

The aqueous carriers useful in the leave-on treatment include water and water solutions of lower alkyl alcohols and polyhydric alcohols. The lower alkyl alcohols useful herein are monohydric alcohols having 1 to 6 carbons, in one aspect, ethanol and isopropanol. The polyhydric alcohols useful herein include propylene glycol, hexylene glycol, glycerin, and propane diol.

### pH

The hair care composition of the present invention may have a pH in the range from about 2 to about 10, at 25° C. More preferably, the hair care composition may have a pH in the range of from about 2 to about 6, alternatively from about 3.5 to about 5, alternatively from about 5.25 to about 7.

Additional Components

The hair care composition described herein may optionally comprise one or more additional components known for use in hair care or personal care products, provided that the additional components are physically and chemically compatible with the essential components described herein, or do not otherwise unduly impair product stability, aesthetics or performance. Such additional components are most typically those described in reference books such as the CTFA Cosmetic Ingredient Handbook, Second Edition, The Cosmetic, Toiletries, and Fragrance Association, Inc. 1988, 1992. Individual concentrations of such additional components may range from about 0.001 wt % to about 10 wt % by weight of the hair care compositions.

Non-limiting examples of additional components for use in the hair care compositions include conditioning agents, natural cationic deposition polymers, synthetic cationic deposition polymers, anti-dandruff agents, particles, suspending agents, paraffinic hydrocarbons, propellants, viscosity modifiers, dyes, non-volatile solvents or diluents (water-soluble and water-insoluble), pearlescent aids, foam boosters, additional surfactants or nonionic cosurfactants, pediculocides, pH adjusting agents, perfumes, preservatives, proteins, skin active agents, sunscreens, UV absorbers, and vitamins.

1. Conditioning Agent

The hair care composition may comprise one or more conditioning agents. Conditioning agents include materials that are used to give a particular conditioning benefit to hair. The conditioning agents useful in the hair care compositions of the present invention typically comprise a water-insoluble, water-dispersible, non-volatile, liquid that forms emulsified, liquid particles. Suitable conditioning agents for use in the hair care composition are those conditioning agents characterized generally as silicones, organic conditioning oils or combinations thereof, or those conditioning agents which otherwise form liquid, dispersed particles in the aqueous surfactant matrix.

One or more conditioning agents are present from about 0.01 wt % to about 10 wt %, from about 0.1 wt % to about 8 wt %, and from about 0.2 wt % to about 4 wt %, by weight of the composition.

Silicone Conditioning Agent

The hair care compositions of the present invention may contain one or more silicone conditioning agents. Examples of the silicones include dimethicones, dimethiconols, cyclic silicones, methylphenyl polysiloxane, and modified silicones with various functional groups such as amino groups, quaternary ammonium salt groups, aliphatic groups, alcohol groups, carboxylic acid groups, ether groups, epoxy groups, sugar or polysaccharide groups, fluorine-modified alkyl groups, alkoxy groups, or combinations of such groups. Such silicones may be soluble or insoluble in the aqueous (or non-aqueous) product carrier. In the case of insoluble liquid silicones, the polymer can be in an emulsified form with droplet size of about 10 nm to about 30 micrometers

Organic Conditioning Materials

The conditioning agent of the compositions of the present invention may also comprise at least one organic conditioning material such as oil or wax, either alone or in combination with other conditioning agents, such as the silicones described above. The organic material can be nonpolymeric, oligomeric or polymeric. It may be in the form of oil or wax and may be added in the formulation neat or in a pre-emulsified form. Some non-limiting examples of organic conditioning materials include, but are not limited to: i) hydrocarbon oils; ii) polyolefins, iii) fatty esters, iv) fluorinated conditioning compounds, v) fatty alcohols, vi) alkyl glucosides and alkyl glucoside derivatives; vii) quaternary ammonium compounds; viii) polyethylene glycols and polypropylene glycols having a molecular weight of up to about 2,000,000 including those with CTFA names PEG-20 200, PEG-400, PEG-600, PEG-1000, PEG-2M, PEG-7M, PEG-14M, PEG-45M and mixtures thereof.

Additional Benefit Agents

The hair care composition may further comprise one or more additional benefit agents. The benefit agents comprise a material selected from the group consisting of anti-dandruff agents, anti-fungal agents, anti-itch agents, anti-bacterial agents, anti-microbial agents, moisturization agents, antioxidants, vitamins, lipid soluble vitamins, perfumes, brighteners, enzymes, sensates, attractants, dyes, pigments, bleaches, and mixtures thereof.

The hair care compositions of the present invention may be presented in typical hair care formulations. They may be in the form of solutions, dispersion, emulsions, powders, talcs, encapsulated, spheres, spongers, solid dosage forms, foams, and other delivery mechanisms.

The hair care compositions may be provided in the form of a porous, dissolvable solid structure, such as those disclosed in U.S. Patent Application Publication Nos. 2009/0232873; and 2010/0179083, which are incorporated herein by reference in their entirety. Accordingly, the hair care compositions comprise a chelant, a buffer system comprising an organic acid, from about 23% to about 75% surfactant; from about 10% to about 50% water soluble polymer; and optionally, from about 1% to about 15% plasticizer; such that the hair care composition is in the form of a flexible porous dissolvable solid structure, wherein said structure has a percent open cell content of from about 80% to about 100%.

The hair care compositions may be in the form of a viscous liquid comprising one or more aptomer, 20% surfactant and a polycarboxylate rheology modifier; wherein the polycarboxylate is specifically chosen to be effective at the high electrolyte levels resulting from the incorporation of the key buffer system and chelant used for this invention. Non-limiting examples include acrylates/C10-C30 alkyl acrylate crosspolymers such as Carbopol EDT2020, 1342,1382, etc. from Lubrizol. Rheology benefits of these actives may include stability, ease of dispensing, smoothness of spreading, etc.

The hair care compositions are generally prepared by conventional methods such as are known in the art of making the compositions. Such methods typically involve mixing of the ingredients in one or more steps to a relatively uniform state, with or without heating, cooling, application of vacuum, and the like. The compositions are prepared such as to optimize stability (physical stability, chemical stability, photostability) and/or delivery of the active materials. The hair care composition may be in a single phase or a single product, or the hair care composition may be in a separate phases or separate products. If two products are used, the products may be used together, at the same time or sequentially. Sequential use may occur in a short period of time, such as immediately after the use of one product, or it may occur over a period of hours or days.

### EXAMPLES

The following examples illustrate non-limiting examples of the invention described herein. The exemplified hair care compositions can be prepared by conventional formulation and mixing techniques. It will be appreciated that other modifications of the hair care compositions within the skill of those in the formulation art can be undertaken without departing from the spirit and scope of this invention. All parts, percentages, and ratios herein are by weight unless otherwise specified. Some components may come from suppliers as dilute solutions. The amount stated reflects the weight percent of the active material, unless otherwise specified.

The following are non-limiting examples of hair care compositions described herein.

Shampoo Composition Examples

| | Shampoo Example | Shampoo Example |
|---|---|---|
| | 1 | 2 |
| Ingredients | wt % | wt % |
| Water Purified | Q.S to 100 | Q.S to 100 |
| Sodium Laureth-3 Sulfate | 21.6 | 21.6 |
| Sodium Lauryl Sulfate | 34.5 | 34.5 |
| Laureth-4 | 0.9 | 0.9 |
| Dimethicone 330M cps | 0.5 | 0.5 |
| Glycol Distearate | 1.5 | 1.5 |
| Polyquaternium-6 | 0.32 | 0.32 |
| H-A1 Aptamer | 0.01 | 0.001 |
| Sodium Benzoate | 0.27 | 0.27 |
| Citric acid 50% Solution | 0.52 | 0.52 |
| Methylchloroisothiazolinone/ | 0.035 | 0.035 |
| methylisothiazolinone | | |
| Sodium chloride | 1.66 | 1.66 |
| Fragrance | 0.65 | 0.65 |
| DL-Panthenol 56% solution | 0.05 | 0.05 |
| Panthenyl Ethyl ether | 0.03 | 0.03 |
| Glycol Distearate | 1.5 | 1.5 |

Additional Shampoo Examples

| | SH | SH | SH | SH | SH | SH | SH |
|---|---|---|---|---|---|---|---|
| Ingredient | Ex 3 | Ex 4 | Ex 5 | Ex 6 | Ex 7 | Ex 8 | Ex 9 |
| Sodium lauryl | | 6 | 10 | 6 | 6 | 9 | |
| ether sulfate | | | | | | | |
| (SLE3S) | | | | | | | |
| Sodium cocoyl | | | | | | | 8.5 |
| isethionate | | | | | | | |
| Sodium lauryl | 1.5 | 7 | 1.5 | 7 | 7 | 6 | |
| sulfate (SLS) | | | | | | | |
| Sodium lauryl | 10.5 | | | | | | |
| ether sulfate | | | | | | | |
| (SLE1S) | | | | | | | |
| Disodium laureth | | | | | | | 8.5 |
| sulfosuccinate | | | | | | | |
| Sodium lauryl | | | | | | | 2.5 |
| sulfoacetate | | | | | | | |
| Sodium Lauroyl | | | | | | | 0.75 |
| Sarcosinate | | | | | | | |
| Cocoamidopropyl | | | | | | | 1.5 |
| Hydroxysultaine | | | | | | | |
| Cocoamidopropyl | 1 | 2 | 2 | 2 | 2 | 2 | 2 |
| Betaine | | | | | | | |
| Coconut mono- | | 0.85 | | 0.85 | | | |
| ethanol amide | | | | | | | |
| (CMEA) | | | | | | | |
| Cetyl alcohol | | | 1 | | | | |
| Stearyl alcohol | | | 2 | | | | |
| Dimethicone | 1 | 1 | 1 | 1 | 1 | | 0.5 |
| Ethylene glycol | 1.5 | 1.5 | 1.5 | 1.5 | 1.5 | | |
| distearate | | | | | | | |
| (EGDS) | | | | | | | |
| Jaguar ® C500¹ | 0.25 | 0.25 | 0.15 | | | | |
| Synthetic Cationic | | | | 0.1 | | | |
| Polymer AMT² | | | | | | | |
| Polydiallyl- | | | | | 0.1 | | |
| dimethyl- | | | | | | | |
| ammonium | | | | | | | |
| chloride | | | | | | | |
| (DADMAC) | | | | | | | |
| H-A1.1 Aptamer | 0.01 | 0.1 | 0.001 | 0.01 | 0.001 | 0.1 | 0.01 |
| Excel Guar³ | | | | | | 0.1 | .15 |
| pH | 6 | 6 | 6 | 6 | 6 | 6 | |
| Water-USP | Q.S. | Q.S. | Q.S. | Q.S. | Q.S. | Q.S. | Q.S. |
| Purified & | to | to | to | to | to | to | to |
| Minors | 100 | 100 | 100 | 100 | 100 | 100 | 100 |

¹Cationic polymer derived from a natural gum with low aqueous viscosity
²Cationic synthetic copolymer
³Cationic plant derived polymer

Rinse-Off Conditioner Formulations

| | Rinse-off | Rinse-off |
|---|---|---|
| | Conditioner | Conditioner |
| | Ex 1 | Ex 2 |
| Ingredients | Wt % | Wt % |
| Amodimethicone 10000 cps | 0.50 | 0.50 |
| Citric acid anhydrous | 0.13 | 0.13 |
| DL-Panthenol 56% solution | 0.054 | 0.054 |
| Panthenyl Ethyl ether | 0.03 | 0.03 |
| Perfume | 0.50 | 0.50 |
| Hydroxypropyl guar (Jaguar HP-105) | 0.350 | 0.350 |
| Quaternium-18 | 0.750 | 0.750 |
| Steramidopropyldimethylamine | 1.00 | 1.00 |
| Gryceryl stearate | 0.25 | 0.25 |
| Cetearyl alcohol and Polysorbate 60 Emulsion ¹ | 0.50 | 0.50 |
| Cetyl alcohol | 1.20 | 1.20 |
| Stearyl alcohol | 0.80 | 0.80 |
| Benzyl alcohol | 0.40 | 0.40 |
| Methylchloroisothiazolinone/ | 0.033 | 0.033 |
| methylisothiazolinone | | |
| H-A1 Aptamer | 0.01 | 0.001 |
| Water Purified | QS to 100 | QS to 100 |

¹ Lipowax P from Lipo (looked in internet)

Additional Examples of Rinse-Off Hair Conditioning Compositions

| | Rinse-off | Rinse-off | Rinse-off | Rinse-off | Rinse-off | Rinse-off |
|---|---|---|---|---|---|---|
| | Condition | Condition | Condition | Condition | Condition | Condition |
| Components | Ex. 3 | Ex. 4 | Ex. 5 | Ex. 6 | Ex. 7 | Ex. 8 |
| Water-USP Purified & | QS to | QS to | QS to | QS to | QS to | QS to |
| Minors | 100% | 100% | 100% | 100% | 100% | 100% |
| BTMS¹ | 2.3 | 2.3 | 2.3 | 2.3 | 2.3 | 2.3 |
| BTMAC² | — | — | — | — | — | — |
| Cetyl alcohol | 1.1 | 1.1 | 1.1 | 1.1 | 1.1 | 1.1 |
| Stearyl alcohol | 2.8 | 2.8 | 2.8 | 2.8 | 2.8 | 2.8 |
| Soy Oligomer³ | 1.0 | — | | — | | — |
| Soy Oligomer Blend⁴ | — | 1.0 | — | | — | |
| Aminosilicone⁵ | 1.0 | 1.0 | 1.0 | 1.0 | 2.0 | 2.0 |
| Perfume | 0.5 | 0.5 | 0.5 | 0.5 | 0.5 | 0.5 |
| Disodium EDTA | 0.1 | 0.1 | 0.1 | 0.1 | 0.1 | 0.1 |
| Panthenol | 0.03 | 0.03 | 0.03 | 0.03 | 0.03 | 0.03 |
| Panthenyl ethyl ether | 0.03 | 0.03 | 0.03 | 0.03 | 0.03 | 0.03 |
| H-A1.1 Aptamer | 0.01 | 0.001 | 0.1 | 0.01 | 0.001 | 0.1 |
| Benzyl Alcohol | 0.4 | 0.4 | 0.4 | 0.4 | 0.4 | 0.4 |
| Preservatives | 0.03 | 0.03 | 0.03 | 0.03 | 0.03 | 0.03 |
| | Rinse-off | Rinse-off | Rinse-off | Rinse-off | Rinse-off | Rinse-off |
| | Condition | Condition | Condition | Condition | Condition | Condition |
| Ingredients | Ex. 9 | Ex. 10 | Ex. 11 | Ex. 12. | Ex. 13 | Ex. 14 |
| Water-USP Purified & | QS to | QS to | QS to | QS to | QS to | QS to |
| Minors | 100% | 100% | 100% | 100% | 100% | 100% |
| BTMS¹ | — | — | — | — | — | — |
| BTMAC² | 2.8 | 2.8 | 2.8 | 2.8 | 2.8 | 2.8 |
| Cetyl alcohol | 1.8 | 1.8 | 1.8 | 1.8 | 1.8 | 1.8 |
| Stearyl alcohol | 4.6 | 4.6 | 4.6 | 4.6 | 4.6 | 4.6 |
| Soy Oligomer³ | — | — | 0.75 | — | — | — |
| Soy Oligomer Blend⁴ | — | 1.0 | — | | — | — |
| Aminosilicone⁵ | 1.0 | — | 0.75 | 1.5 | 2.0 | 2.0 |
| Perfume | 0.5 | 0.5 | 0.5 | 0.5 | 0.5 | 0.5 |
| Disodium EDTA | 0.1 | 0.1 | 0.1 | 0.1 | 0.1 | 0.1 |
| Panthenol | 0.03 | 0.03 | 0.03 | 0.03 | 0.03 | 0.03 |
| Panthenyl ethyl ether | 0.03 | 0.03 | 0.03 | 0.03 | 0.03 | 0.03 |
| H-A1 Aptamer | 0.01 | 0.001 | 0.1 | 0.010 | 0.001 | 0.1 |
| Benzyl Alcohol | 0.4 | 0.4 | 0.4 | 0.4 | 0.4 | 0.4 |
| Preservatives | 0.03 | 0.03 | 0.03 | 0.03 | 0.03 | 0.03 |
| | | | Rinse-off | Rinse-off | Rinse-off | Rinse-off |
| | | | Condition | Condition | Condition | Condition |

| Ingredients | Ex. 15 | Ex. 16 | Ex. 17 | Ex. 18 |
|---|---|---|---|---|
| Water-USP Purified & | QS to | QS to | QS to | QS to |
| Minors | 100% | 100% | 100% | 100% |
| BTMS¹ | 3.76 | 3.76 | 3.76 | 3.76 |
| BTMAC² | — | — | — | — |
| Cetyl alcohol | 1.3 | 1.3 | 1.3 | 1.3 |
| Stearyl alcohol | 3.2 | 3.2 | 3.2 | 3.2 |
| Soy Oligomer³ | 1.0 | 1.0 | — | — |
| Soy Oligomer Blend⁴ | — | — | — | — |
| Aminosilicone⁵ | — | — | 1.0 | 1.0 |
| Perfume | 0.5 | 0.5 | 0.5 | 0.5 |
| Disodium EDTA | 0.1 | 0.1 | 0.1 | 0.1 |
| Panthenol | — | — | — | — |
| Panthenyl ethyl ether | — | — | — | — |
| H-A1.1 Aptamer | 0.01 | 0.001 | 0.1 | 0.01 |
| Benzyl Alcohol | 0.4 | 0.4 | 0.4 | 0.4 |
| Preservatives | 0.03 | 0.03 | 0.03 | 0.03 |
| Disposition Aid | 0.5 | — | 0.5 | — |
| polymer⁶ | | | | |

¹Behenyltrimethylammonium methylsulfate, from Feixiang
²Behenyltrimethylammonium chloride, Genamin KDMP, from Clariant
³HY-3050, from Dow Corning
⁴HY-3051, from Dow Corning
⁵Y-14945; 10,000 cps aminodimethicone, from Momentive
⁶ABC1459 from Mitsubishi Chemical

Examples of Leave-on Treatment (LOT) Compositions

| | LOT | LOT | LOT |
|---|---|---|---|
| Components | Ex. 1 | Ex. 2 | Ex. 3 |
| Dipropyleneglycol | 0.500 | 0.500 | 0.500 |
| Monomethylether | | | |
| Disodium Ethylene diamine | 0.141 | 0.141 | 0.141 |
| diacetic acid | | | |
| PEG-40 Hydrogenated Castor Oil | 0.500 | 0.500 | 0.500 |
| Polysorbate 80¹ | 0.120 | 0.120 | — |
| Amodimethicone and | 1.810 | 1.810 | 1.928 |
| Cetrimonium Chloride | | | |
| Polyquaternium 11² | 1.335 | 1.335 | 1.335 |
| Citric Acid Anhydrous | 0.080 | 0.080 | 0.20 |
| 2-Amino-2-methyl-1-propanol | 0.100 | 0.100 | 0.100 |
| DMDM Hydantoin (55%)³ | 0.370 | — | — |
| Benzyl Alcohol | — | 0.400 | 0.4 |
| Neolone 950 Preservative⁴ | — | 0.053 | 0.053 |
| Perfume | 0.200 | 0.200 | 0.10 |
| H-A1 Aptamer | 0.01 | 0.0010.1 | 0.1 |
| Water-USP Purified & Minors | QS to 100% | QS to 100% | QS to 100% |

¹Nonionic surfactant and emulsifier derived from polyethoxylated sorbitan and oleic acid
²Copolymer of vinylpyrrolidone and quaternized dimethylaminoethyl methacrylate
³1,3-Bis(hydroxymethyl)-5,5-dimethylimidazolidine-2,4-dione
⁴Preservative containing Methylisothiazolinone

### V. Examples

### Example 1. Aptamers Design

A. Library Preparation

A DNA library of about 10¹⁵ different sequences (10 nmoles), containing a random region of 40 nucleotides flanked by two conserved regions, i.e. a 5′ forward primer recognition sequence (5′-AACTACATGGTATGTGGTGAACT-3′) and a 3′ reverse primer recognition sequence (5′-GACGTACAATGTACCC-3′), is solubilized in 100 μL of H₂O and split into aliquots of 16.6 μL (about 1.66 nmoles of DNA). To each library aliquot, 50 μL of 10× selection buffer (100 mM HEPES, 1.2 M NaCl, 50 mM KCl, 50 mM MgCl2; pH 8.2) and 383.4 μL of H₂O are added.

The library solution is then heated for 10 minutes at 95° C. and immediately placed in an iced ethanol bath for 15 min. Finally, the library is incubated at room temperature for 10 minutes, producing the snap cooled library used during selection. When needed, 50 μL of a commercial volume shampoo is added to the library.

B. Hair Samples Characterization

The hair is sourced from Caucasian women as ponytails of length ˜30 cm from International Hair Importers (New York, USA). Prior to use for Aptamer selection each ponytail is washed three times in Pantene Silky Smooth Shampoo and Conditioner sourced from Japan. The shampoo is added at 0.1 g shampoo per g of hair and milked for 30 secs into the hair. This is then rinsed for 30 secs and repeated. The conditioner is also added at 0.1 g conditioner per g of hair, milked for 30 secs and rinsed for 30 secs. This completed one complete cycle and is repeated three times. The hair is then left to dry overnight.

Each ponytail is also characterized at the root and tip end to assess chemical and physical damage. All ponytails are from women who had done no coloring, perming or relaxing treatments but had been exposed to physical damage (washing, brushing etc) and UV exposure. The cuticle quality is measured at root and tip using SEM (Scanning Electron Microscopy). Fifty fibers are assessed on a scale of 0 to 5 where 0=no cuticle damage, 1=little cuticle damage, 3=high cuticle damage, 5=stripped cuticle. The score from each fiber is then added to give a total damage score (maximum score=100). The degree of oxidative damage is measured at root and tip using FT-IR measurement of the cuticle cysteic acid. This method has been established to be suitable for studying the effects of oxidative treatments on hair by quantifying the amount of cysteic acid that is produced from oxidation of cystine (Strassburger, J., J. Soc. Cosmet. Chem., 36, 61-74 (1985); Joy, M. & Lewis, D. M., Int. J. Cosmet. Sci., 13, 249-261 (1991); Signori, V. & Lewis, D. M., Int. J. Cosmet. Sci., 19, 1-13 (1997)). A Perkin Elmer Spectrum® 1 Fourier Transform Infrared (FTIR) system equipped with a diamond Attenuated Total Internal Reflection (ATR) cell is used to measure the cysteic acid concentration in human hair. Four readings per switch are taken (˜⅓ and ⅔s down the switch on both sides), and an average calculated. As prescribed by Signori & Lewis in 1997, a normalized double derivative analysis routine is used. The original spectra are initially converted to absorbance, before being normalized to the 1450 cm⁻¹ band (the characteristic and invariant protein CH₂ stretch). This normalized absorbance is then twice derivatised and the absorbance at 1040 cm⁻¹ is taken as the relative concentration of cysteic acid.

C. Aptamer Selection

Aptamer selection used one library aliquot containing about 10¹⁵ sequences. To this library aliquot, 50 μL of 10× selection buffer (100 mM HEPES, 1.2 M NaCl, 50 mM KCl, 50 mM MgCl2; pH 8.2) and 383.4 μL of H₂O are added. The library solution is snap cooled by heating the library for 10 minutes at 95° C. and immediately placing the solution in an iced ethanol bath for 15 min. Finally, the library is incubated at room temperature for 10 minutes, producing the snap cooled library used during selection. After the initial snap cooling of the library, an aliquot of 50 μL of a commercial volume shampoo (clarifying shampoo, silicone free) is added to the library.

Aptamer selection is performed on hair samples dipped into a solution containing the aptamer library. In the first selection round, a 3 cm long lock of hair held together by an elastic band and weighing approximately 0.03 g is placed into the snap cooled library solution. The hair is incubated in the library solution for 20 minutes at room temperature. After incubation, the hair is removed and placed into a fresh 2 mL plastic Eppendorf tube containing 1 mL of selection buffer (100 mM HEPES, 1.2 M NaCl, 50 mM KCl, 50 mM MgCl2; pH 8.2) and placed on a rotator for 5 minutes. The hair is removed from the binding buffer and placed in a fresh 2 mL Eppendorf tube containing 1 mL of fresh selection buffer (100 mM HEPES, 1.2 M NaCl, 50 mM KCl, 50 mM MgCl2; pH 8.2) and is placed on a rotator for 5 minutes, resulting in two washes total. To remove sequences that have successfully bound to the hair sample, the washed hair sample is then placed into a 2 mL Eppendorf tube containing 500 μL of 6 M Urea and incubated at 85° C. for 10 minutes. After heating, this first elution solution is recovered. The hair is then placed into a fresh 2 mL Eppendorf tube containing 500 μL of fresh 6 M Urea and the sample is heated at 85° C. for 10 minutes. This second elution solution is recovered and combined with the first elution solution. The DNA from the combined solution are purified using the GeneJET PCR Purification Kit (ThermoFisher Scientific, catalog # K0702) following manufacturer's instructions.

Purified DNA underwent a test PCR in which library aliquots are amplified for an increasing number of cycles to determine the optimum number of cycles to give a 1.5 ng band on a 10% Polyacrylamide Gel. PCR is conducted using Standard Taq Polymerase Buffer (New England BioLabs, Catalog # B9014S), deoxyribonucleotide (dNTP) solution mix (New England BioLabs, Catalog # N0447L), 10 μM forward primer (5′-AACTACATGGTATGTGGTGAACT-3′) (TriLink, Catalog # NA), 10 μM reverse Primer (5′-GACGTACAATGTACCC-3′) (TriLink, Catalog # NA), and taq polymerase (New England BioLabs, Catalog # M0273X). Once the optimum number of cycles is determined, the library from the first selection round is amplified and purified with the GeneJET PCR Purification Kit (ThermoFisher Scientific, Catalog # K0702).

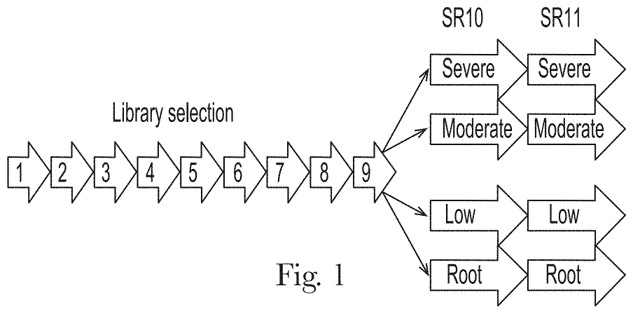

After the first selection round, the library is split into two channels. In channel B, selection is performed against tips of hair alone. In channel A, for each selection round, counter selection is performed against a clutch of hair from near the root, followed by positive selection against tip hair of the same sample. After 9 rounds of selection, the libraries from each channel are further split in 4 aliquots. Then, these sub-libraries are used to perform two more positive selection rounds against hair that is either severely damaged, moderately damaged, lightly damaged, or undamaged (root hair), as illustrated in FIG. 1.

During each positive selection experiment, a lock of hair (length 3 cm, weight about 0.03 g) held together by an elastic band is placed into a 2 mL tube containing an aliquot of snap cooled library solution (500 μL), ensuring that the hair sample is fully submerged. The sample is incubated at room temperature for 20 minutes. The lock of hair is then removed, place into a new 2 mL tube containing 1 mL of 1× selection buffer (10 mM HEPES, 120 mM NaCl, 5 mM KCl, 5 mM MgCl2; pH 7.4) and mixed for 5 minutes using a rotator to wash and remove unbound sequences from the hair sample. This cleaning step is repeated one more time. Next, the hair lock is placed into a new 2 mL tube containing 500 μL of 6 M Urea and incubated at 85° C. for 10 minutes to elute the bound sequences. This elution process is repeated and the two elution solutions are combined (1000 μL in total). The eluted library is cleaned up with a GeneJET PCR Purification Kit (ThermoFisher Scientific, Catalog # K0702), following manufacturer's instructions.

After each positive selection, the purified library is subjected to a test PCR where 5 μL of recovered library is PCR-amplified in increasing cycles to determine the optimum number of cycles through visualization on a 10% polyacrylamide gel (see table 1). The PCR reactions are preformed using Standard Taq Polymerase Buffer (New England BioLabs, Catalog # B9014S), deoxyribonucleotide (dNTP) solution mix (New England BioLabs, Catalog # N0447L), 10 μM forward primer (5′-AACTACATGGTATGTGGTGAACT-3′) (TriLink, Catalog # NA), 10 μM reverse Primer (5′-GACGTACAATGTACCC-3′) (TriLink, Catalog # NA), and taq polymerase (New England BioLabs, Catalog # M0273X). Once the optimal number of cycles is determined, the full library is PCR amplified in several reaction tubes to produce the desired amount of DNA for the next selection round. Over the selection process, the amount of DNA library carried forward in each selection round is decreased by reducing the number of reaction tubes until a minimum of five reaction tubes is reached, increasing the aptamer selection stringency (see table 1).

The product of the PCR reactions is purified using the GeneJET PCR Purification Kit (ThermoFisher Scientific, Catalog # K0702). This library is then transcribed using T7 RNA Polymerase with RNAPol Reaction Buffer (New England BioLabs, Catalog # M0251), Ribonucleotide Solution Set (New England BioLabs, Catalog # N0450), and RNase Inhibitor, Murine (New England BioLabs, Catalog # M0314). The DNA template and transcription solution are mixed and incubated for 16 hours at 37° C. Transcription creates RNA that is antisense to the selected library. Following transcription, the DNA template is digested using DNase I (New England BioLabs-M0303, Canada). Then, the RNA is purified using the RNeasy MinElute Cleanup Kit (Qiagen, Catalog #74204). RNA yield is calculated using the A260 value, and the desired amount of RNA s reverse transcribed using the M-MuLV Reverse Transcriptase kit (New England BioLabs, Catalog # M0253) as well as 100 μM forward primer (5′-AACTACATGGTATGTGGTGAACT-3′) (TriLink, Catalog # NA), a deoxyribonucleotide (dNTP) solution mix (New England BioLabs, Catalog # N0447), and RNase inhibitor (New England BioLabs, Catalog # M0314). To remove the remaining RNA template, the reverse transcription solution is carried forward into an RNase H reaction using an RNase H reaction kit (New England BioLabs, Catalog # M0297L), after which the solution is purified using the GeneJET PCR Purification Kit (ThermoFisher Scientific, Catalog # K0702). After purification, the produced single stranded sense DNA is used in the following selection round.

During each counter-selection experiment, a pre-washed sample of hair is rinsed with three successive applications of 1 mL sterile HPLC-grade water. The library solution (500 μL) and selection buffer (10 mM HEPES, 120 mM NaCl, 5 mM KCl, 5 mM MgCl2; pH 7.4) are pipetted into a tube and about 1 cm clutch of the hair is submerged into the tube. The sample is incubated at 50 rpm, 37° C., for 20 minutes. The clutch of hair is removed and placed in a 2 mL tube containing 1 mL of 1× selection buffer (10 mM HEPES, 120 mM NaCl, 5 mM KCl, 5 mM MgCl2; pH 7.4) and placed on a rotator at 50 rpm for 5 minutes. This wash is repeated 1 additional time. The solution containing unbound DNA are combined and cleaned up with a GeneJET PCR Purification Kit (ThermoFisher Scientific, Catalog # K0702), followed by preparation for the positive selection experiment as described above (Example 1. Aptamer Design, A. Library Preparation).

Table 1 shows how aptamer selection progressed, the number of PCR cycles required to recover the aptamer library following the completion of a selection round, as well as how selection stringency increased between selection rounds by decreasing the number of reaction tubes and therefore the amount of library carried forward.

TABLE 1
Summary of aptamer selection for hair root and hair tip

| | | | | | | Number |
|---|---|---|---|---|---|---|
| | | | | | | of |
| | | | | | | reaction |
| | | | | | | tubes |
| | | | | | | used to |
| | | Channel A | | Channel B | | amplify |
| | | Number | | Number | | selection |
| Selec- | | of | Counter | of | Counter | library |
| tion | Hair | PCR | Selec- | PCR | Selec- | in each |
| Round | Sample | cycles | tion | cycles | tion | channel |
| 1 | Hair #18 | 6 | N/A | N/A | N/A | |
| 2 | Hair #221A | 18 | None | 18 | N/A | 36 |
| 3 | Hair #12H | 17 | None | 17 | N/A | 20 |
| 4 | Hair #14C | 15 | None | 15 | N/A | 10 |
| 5 | Hair 17A | 18 | root of | 12 | N/A | 5 |
| | | | same | | | |
| | | | hair | | | |
| | | | sample | | | |
| 6 | Hair #18 | 12 | root of | 17 | N/A | 5 |
| | | | same | | | |
| | | | hair | | | |
| | | | sample | | | |
| 7 | Hair #221A | 19 | root of | 19 | N/A | 5 |
| | | | same | | | |
| | | | hair | | | |
| | | | sample | | | |
| 8 | Hair #12A | 19 | root of | 19 | N/A | 5 |
| | | | same | | | |
| | | | hair | | | |
| | | | sample | | | |
| 9 | Hair #14C | 16 | root of | 16 | N/A | 5 |
| | | | same | | | |
| | | | hair | | | |
| | | | sample | | | |

Following the completion of 9 rounds of selection in both channel A and channel B, the recovered library from selection round 9 is amplified and split equally into 4 aliquots. These split libraries are assigned to one of the following split sub-channels in each channel: severely damaged, moderately damaged, lightly damaged, and undamaged (root) hair samples. In the same manner that previous selection rounds are conducted, the split rounds are carried out. Selection split rounds are performed as outlined in Table 2, where aptamers are selected based on their ability to bind to severely damaged, moderately damaged, lightly damaged, or undamaged (root hair) samples.

TABLE 2
Split selection rounds for severely damaged, moderately damaged, lightly damaged, or
undamaged (root hair) samples and the corresponding number of PCR cycles required to obtain a
1.5 ng band on a 10% polyacrylamide gel.

| Split | Channel A | | | | Channel B | | | |
|---|---|---|---|---|---|---|---|---|
| Selection | Severe | Moderate | Light | Undamaged | Severe | Moderate | Light | Undamaged |
| Round | Damage | Damage | Damage | (Root) | Damage | Damage | Damage | (Root) |
| 10 | 8 PCR | 8 PCR | 5 PCR | 10 PCR | 12 PCR | 15 PCR | 14 PCR | 18 PCR |
| | cycles | cycles | cycles | cycles | cycles | cycles | cycles | cycles |
| 11 | 14 PCR | 15 PCR | 12 PCR | 14 PCR | 14 PCR | 14 PCR | 14 PCR | 16 PCR |
| | cycles | cycles | cycles | cycles | cycles | cycles | cycles | cycles |

D. Aptamers Sequencing

The selected libraries 7 to 9 in each channel as well as all the split selections against specific hair types are prepared for next generation sequencing (NGS) through a two-step PCR process. In the first step, a different hex code (6 base sequence) and a portion of a universal sequencing primer is added to the 5′ end of each aptamer library. In the second step, complete universal sequencing primers are added to both ends. After the second PCR step, the libraries are purified through acrylamide electrophoresis and balanced for relative quantity. These libraries are then pooled and sent to the Hospital for Sick Children in Toronto for NGS with an Illumina HiSeq instrument.

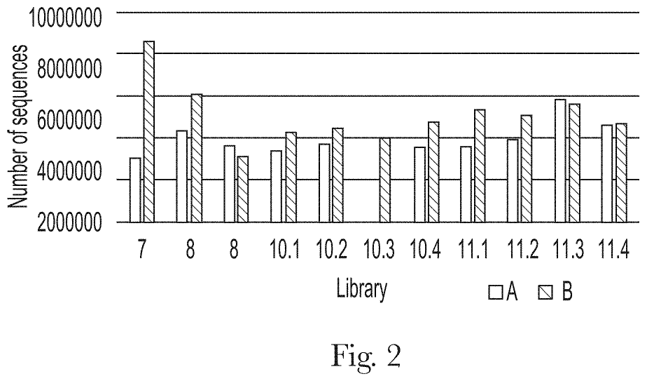

The sequencing data is tabulated and analyzed. A total of 96,464,333 sequences are analyzed and each library contained more than 2,000,000 different sequences (see FIG. 2). The sequences from selection round 9 within each channel are sorted by copy number and named in descending order with the highest copy number sequence being named H-A1 for channel A and H-B1 for channel B. These top sequences are listed in Table 1.

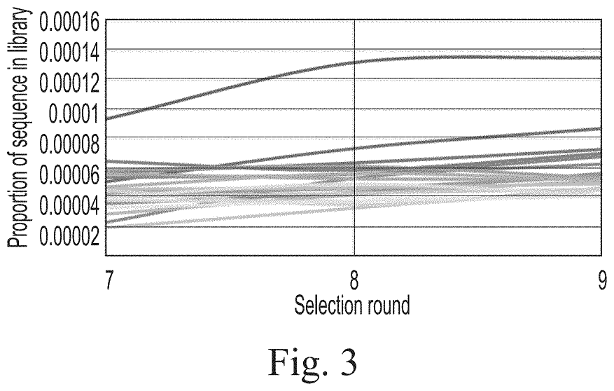

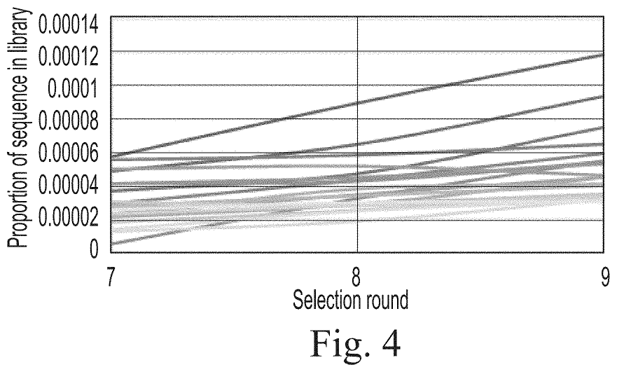

For each channel, the copy numbers of the top sequences of selection round 9 (Table 1) are determined on the libraries obtained from the other selection rounds. Finally, the frequency is computed for each sequence by dividing observed copy number by the total number of sequences observed in the particular library. Enrichment trajectories of the top 20 sequences in terms of frequency across different selection rounds are plotted (see FIGS. 3 and 4).

In FIGS. 3 and 4, it is clear that the top sequence in terms of abundance in selection round 9 is substantially more enriched than the other sequences. Furthermore, there appears to be two types of trajectories, one that increases from round 7 to 8 and then plateaus, and one that is relatively level over all three selection rounds.

### Example 2. Covariance Analysis of Sequences

A covariance analysis for the change in sequence frequency is performed on the top 100 aptamers of channels A and B. First, for each selection round, the frequency data is normalized by dividing the observed frequency of each aptamer by the average of the frequencies of the top 100 aptamers. This normalization allowed eliminating potential differences caused by PCR amplification prior to NGS analysis among different selection rounds. Then, the normalized values of each aptamer in selection round 7 are subtracted from the normalized values of the corresponding aptamer in selection rounds 8 to 11. The resulting matrix is used for the correlation analysis.

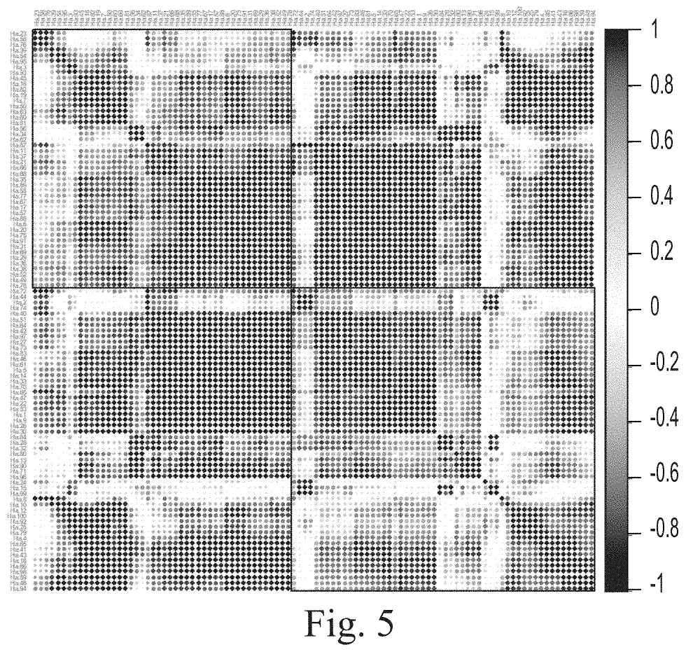

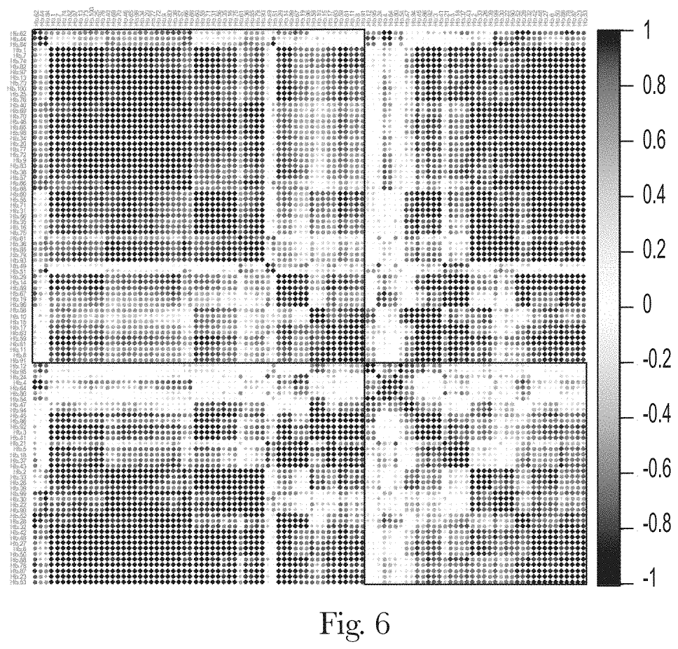

A Pearson correlation across the selection rounds is performed. Since a different sample of hair is used in each selection round, it is reasonable to assume that the covariance among aptamer frequencies would be due to covariance in the abundance of the epitope within the sample of hair that they bind to. Thus, each cluster of covarying aptamers corresponds to a group of aptamers that bind to a different epitope within the hair. An Euclidean distance matrix from the correlation matrix is generated and used as the basis for clustering with a Ward.D2 algorithm (see FIGS. 5 and 6). These analyses are performed with the software R. The order of the aptamers in FIGS. 5 and 6 is the same on the x as the y axis, thus there is a correlation of +1.0 along the diagonal (dark blue). Based on this analysis, at least two different epitopes are likely the binding sites of the selected aptamers.

### Example 3. Aptamers Binding

Four aptamers (H-A1, H-A2, H-B1, and H-B2) are synthesized (Integrated DNA Technologies, Inc.) with a HEX fluorophore on the 5′ end and dissolved to a final concentration of 1 μM in water as a stock solution.

Five hair samples (2.5 mg of the tips, 3 cm in length) are incubated against each of the four aptamers at 50 nM in 1 mL of 1× selection buffer (10 mM HEPES, 120 mM NaCl, 5 mM KCl, 5 mM MgCl2; pH 7.4) at room temperature for 30 minutes. The supernatant is removed and collected. Then, the hair samples are washed with 1× selection buffer twice to remove any unbound aptamer and the supernatant is collected. Bound aptamers are then eluted by incubating the hair samples in a 6 M urea solution at 85° C. for 10 minutes. The amount of eluted aptamer is quantified by fluorescence spectroscopy (excitation λ=535 nm, emission λ=555 nm). This assay showed that H-A1 and H-B2 performed consistently better than the other two aptamers.

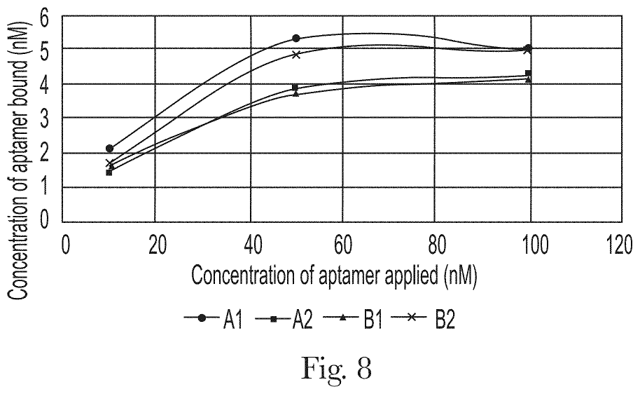

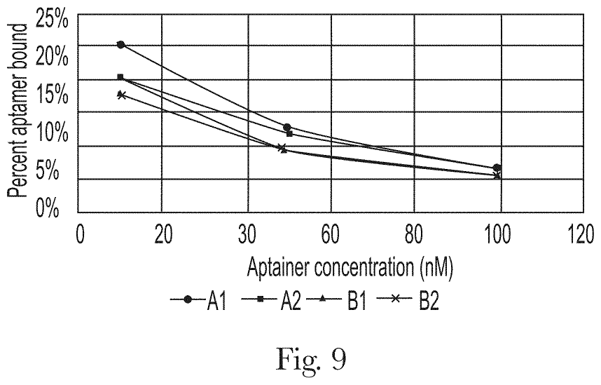

After identifying the best performing aptamers, solutions of different concentrations of the aptamers are tested to identify the saturation point for binding to the hair. Solutions of 10 nM, 50 nM, and 100 nM aptamer in 1× selection buffer (10 mM HEPES, 120 mM NaCl, 5 mM KCl, 5 mM MgCl2; pH 7.4) are incubated with hair samples using the same method described above. Amounts between 5% an 15% (in molar basis) of H-A1 and H-B2 aptamers are bound to the hair tip samples. Based on this analysis, it is clear that the saturation concentration is about 50 nM for 2.5 mg of hair or 20 nM/mg (see FIG. 8) and that a higher proportion of the aptamer is bound at a lower concentration (see FIG. 9).

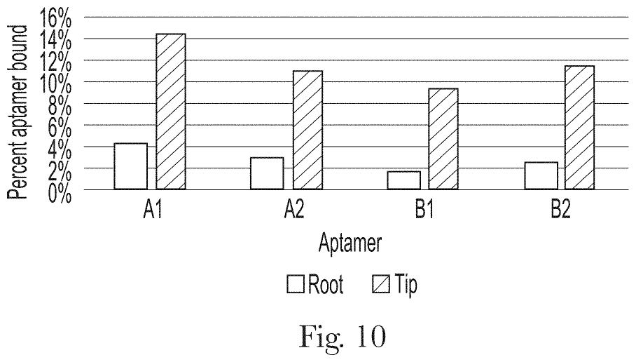

Finally, the preferential binding capacity of these aptamers to tip hair over root hair is confirmed (see FIG. 10). The analysis is performed with solutions of aptamer at 50 nM and hair sample #18.

### Example 4. Motif Analysis

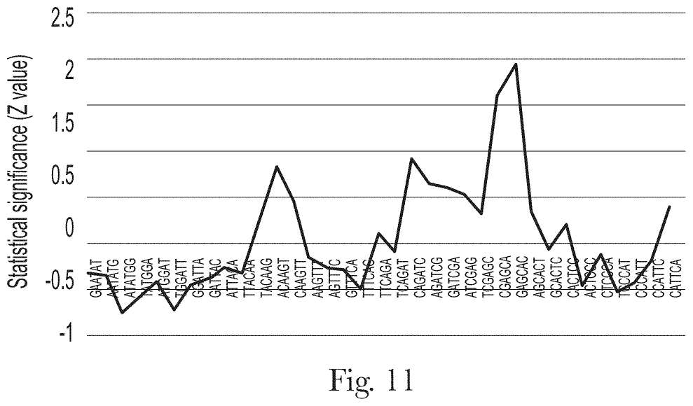

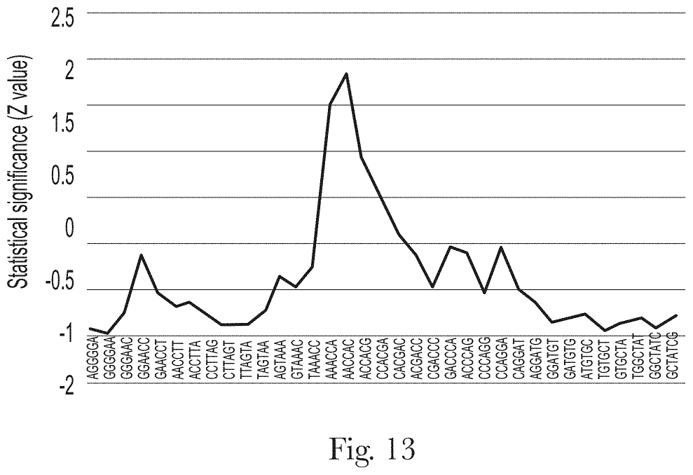

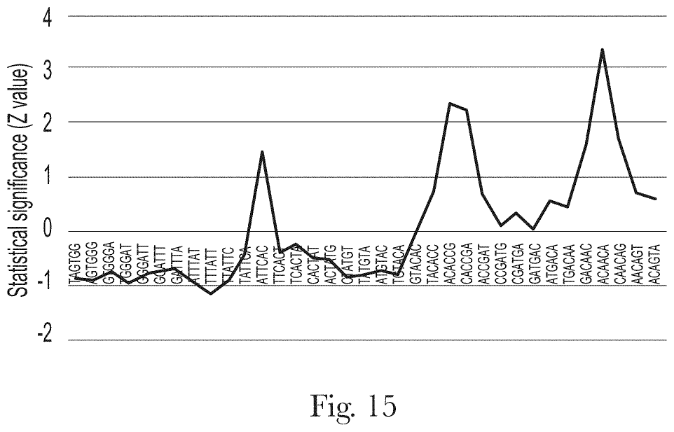

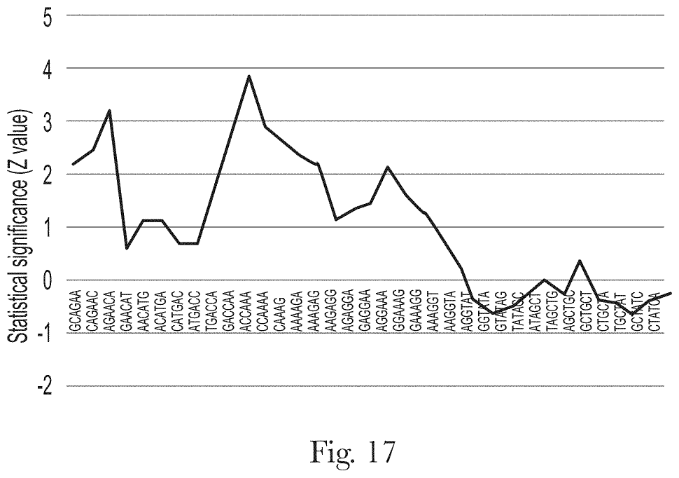

The frequency of motifs of six nucleotides from the random regions of the top four aptamers (H-A1, H-A2, H-B1, and H-B2) within all the sequences of selection round 11 library (highly damaged hair only) is determined. Then, the average motif frequency is subtracted from the frequency of each motif and this value is divided by the standard deviation of all the motifs frequencies in that selection round, resulting in a Z value for every motif (see FIGS. 11, 13, 15, and 17). It stands to reason that sequences containing high frequency motifs may also bind to damaged hair.

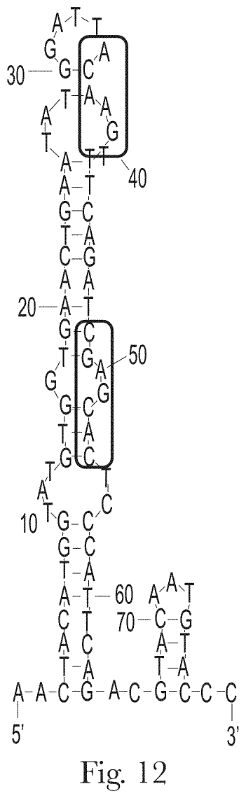

The prediction of the secondary structures of the aptamers is performed with DINAmelt (http://unafold.rna.albany.edu/?q=DINAMelt/Quickfold) and the motifs are highlighted within these structures (see FIGS. 12, 14, 16, and 18).

A. Analysis of Random Region of Aptamer H-A1:

The motifs:

SEQ ID NO 201:

5′-CGAGCAC-3′

SEQ ID NO 202:

5′-ACAAGT-3′

from the variable region of aptamer H-A1 (SEQ ID NO 1):

5′-GAATATGGATTACAAGTTTCAGATCGAGCACTCCCATTCA-3′

are found at a significantly higher frequency than would be expected randomly. This means that these particular motifs are positively selected for within this hair based aptamer selection process. Any sequences containing these motifs may also be expected to bind to damaged hair.

FIG. 12. The predicted secondary structures of aptamer H-A1 and its conserved motifs.

B. Analysis of Random Region of Aptamer H-A2:

The motif:

SEQ ID NO 203:

5′-AAACCACGAC-3′

from the variable region of aptamer H-A2 (SEQ ID NO 2):

5′-AGGGGAACCTTAGTAAACCACGACCCAGGATGTGCTATCG-3′

is found at a significantly higher frequency than would be expected randomly. This means that this particular motif is positively selected for within this hair based aptamer selection process. Any sequences containing this motif may also be expected to bind to damaged hair.

C. Analysis of Random Region of Aptamer H-B1:

The motifs:

SEQ ID NO 204:

5′-ATTCAC-3′

SEQ ID NO 205:

5′-ACACCGA-3′

SEQ ID NO 206:

5′-GACAACAG-3′

SEQ ID NO 207:

5′-ACACCGANGACAACA-3′

from the variable region of aptamer H-B1 (SEQ ID NO 101):

5′-TAGTGGGATTTATTCACTATGTACACCGATGACAACAGTA-3′

wherein N stands for any nucleotide, are found at a significantly higher frequency than would be expected randomly. This means that these particular motifs are positively selected for within this hair based aptamer selection process. Any sequences containing any of these motifs may also be expected to bind to damaged hair.

D. Analysis of Random Region of Aptamer H-B2:

The motif:

SEQ ID NO 208:

5′-GCAGAACA-3′

SEQ ID NO 209:

5′-AACATGA-3′

SEQ ID NO 210:

5′-TGACCAAAAGAGGAAAGG-3′

SEQ ID NO 211:

5′-AAGAGGAAAGG-3′

SEQ ID NO 212:

5′-GCAGAACATGACCAAAAGAGGAAAGG-3′

from the variable region of aptamer H-B2 (SEQ ID NO 102):

5′-GCAGAACATGACCAAAAGAGGAAAGGTATAGCTGCTATCA-3′

are found at a significantly higher frequency than would be expected randomly. This means that these particular motifs are positively selected for within this hair based aptamer selection process. Any sequences containing these motifs are may also be expected to bind to damaged hair.

E. Analysis of Common Motifs within Aptamer Library:

A search for common motifs within the top 10,000 sequences in terms of frequency from channels A and B is performed. The lead motif identified in terms of significant deviation from random distribution is SEQ ID NO 213.

SEQ ID NO 213:

5′-AACCACAA-3′

As an example, this motif is found in the following sequence, in which the 5′- and 3′-primer recognition sequences are eliminated for simplicity. Oligonucleotides may comprise the motif SEQ ID NO 213.

SEQ ID NO 150, H-B50:
5′-GGCCCTGTATAAAGATTCGACTCTGTCAACCACAAACCTA-3′

### Example 5. Analysis of Sequences Similarity

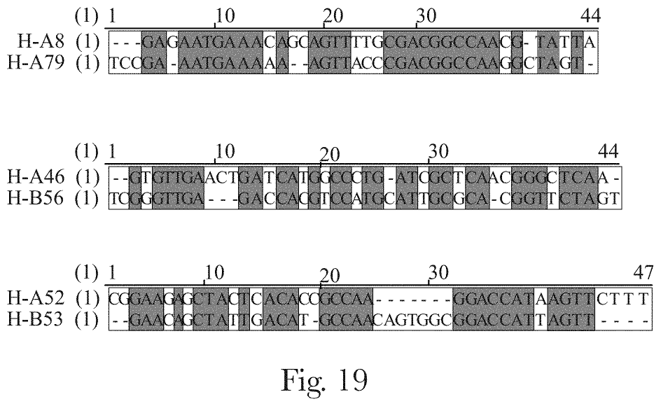

Alignment of SEQ ID NO 1 to SEQ ID NO 200 is performed using the software Align X, a component of Vector NTI Advanced 11.5.4 by Invitrogen. Several groups of sequences have at least 60% or at least 50% nucleotide sequence identity as illustrated in the alignments of FIG. 19. In these alignments, only the central variable region of the aptamers is included for simplicity. Thus, oligonucleotides with at least 50% or at least 60% nucleotide sequence identity to sequences may be selected from the group consisting of SEQ ID NO 1 to SEQ ID NO 200.

### Example 6. Truncation of Aptamers

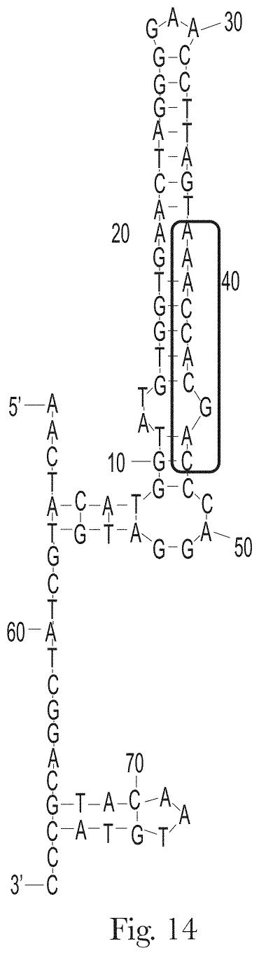

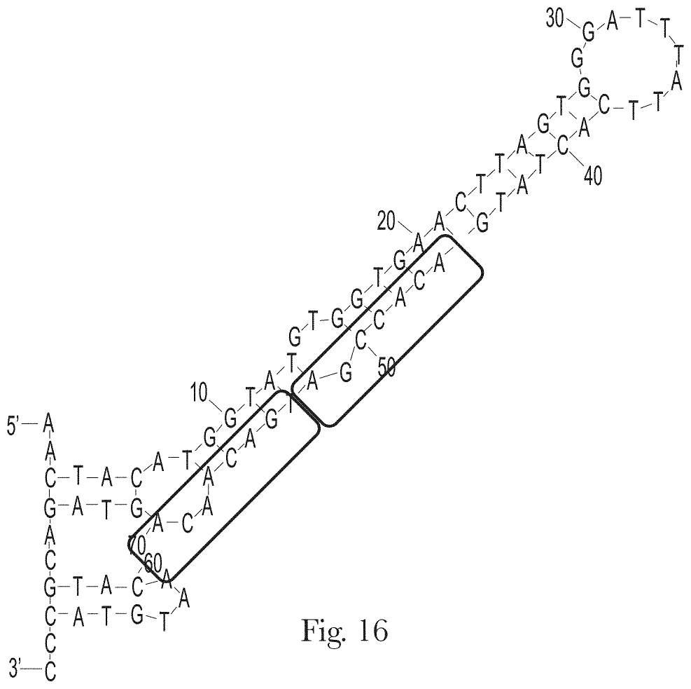

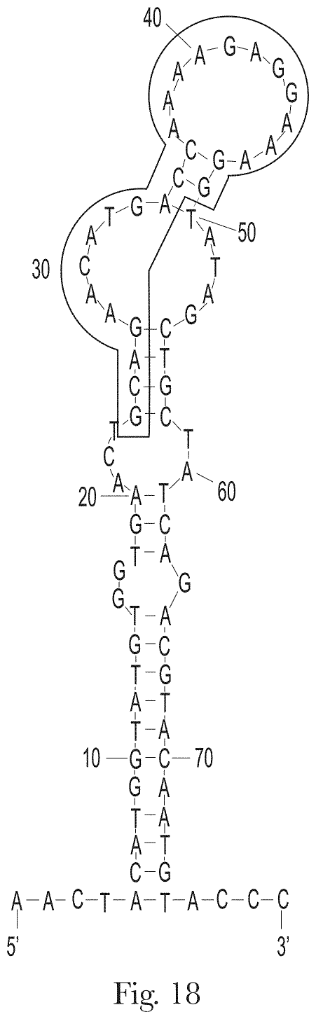

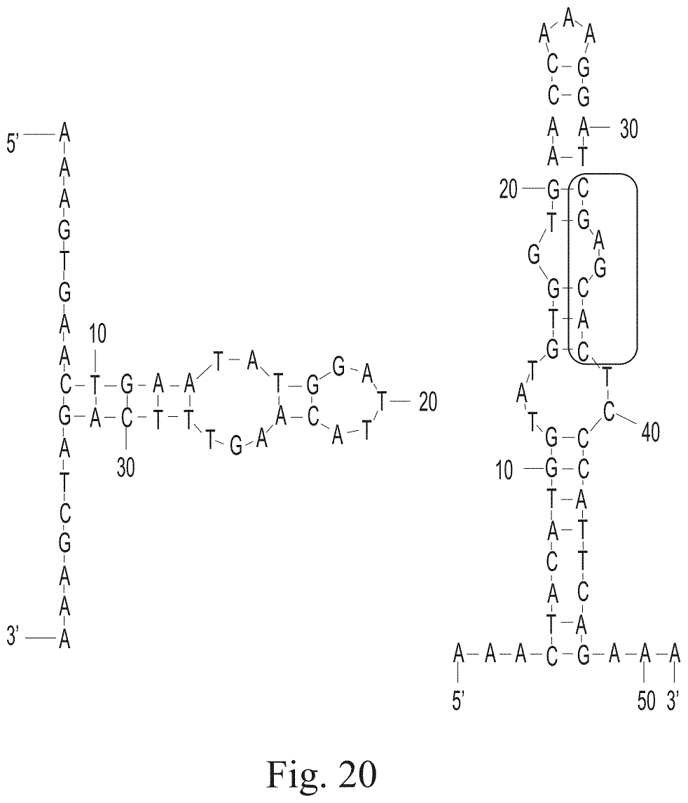

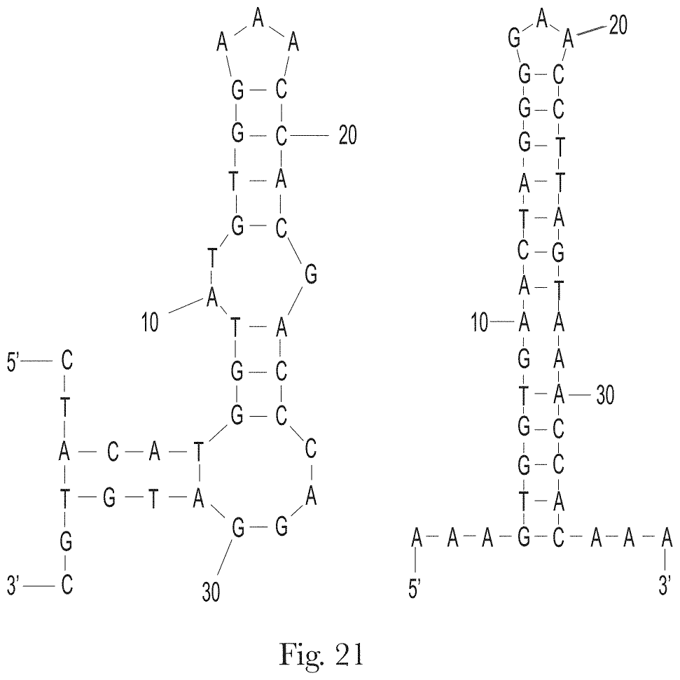

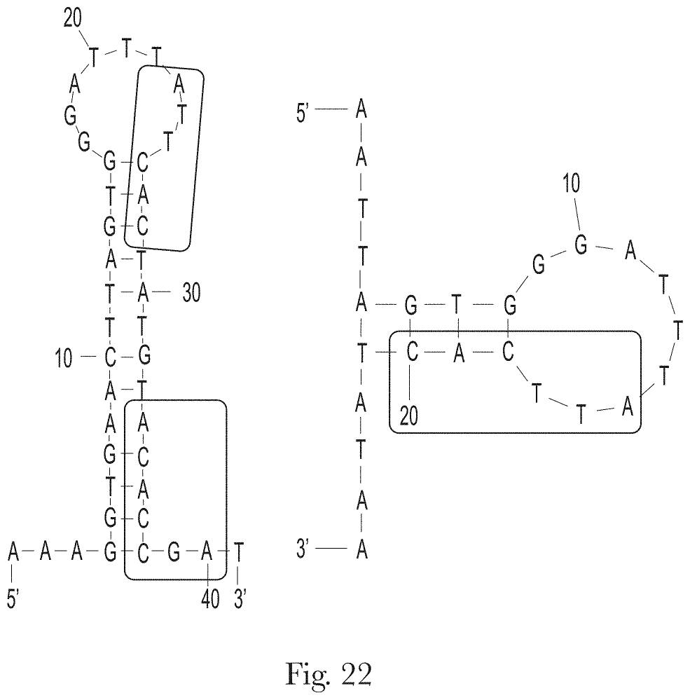

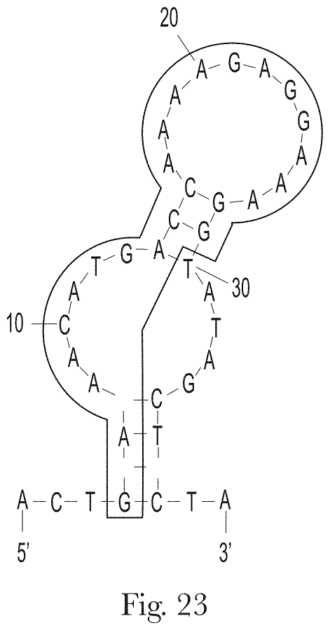

Starting from the predicted secondary structure of the top aptamers (H-A1, H-A2, H-B1, and H-B2), smaller oligonucleotides comprising some of the secondary structure elements are designed. For instance, aptamers H-A1.1 and H-A1.2 are derived from aptamer H-A1 (see FIG. 12). H-A1.1 comprises the top portion of the structure, while HA-1.2 comprises most of the secondary structure (see FIG. 20). Aptamers H-A2.1 and H-A2.2 are derived from aptamer H-A2 (see FIG. 14). H-A2.1 comprises the middle portion of the structure, while H-A2.2 comprises the top of the structure (see FIG. 21). Aptamers H-B1.1 and H-B1.2 are derived from aptamer H-B1 (see FIG. 16) and comprise the top portion of the structure (see FIG. 22). Aptamer H-B2.1 is derived from aptamer H-B2 (see FIG. 18) and comprise the top portion of the structure (see FIG. 23).

Table 3 provides binding results for each of these truncated aptamers with three hair samples. Hair sample #26 is analyzed twice with all truncated aptamers because this sample provided the highest binding affinity. These binding assays are performed and analyzed in a manner identical to that described previously for the full-length aptamers (see Example 3).

TABLE 3
Percent of truncated aptamers bound to different hair samples.

| | Hair sample | | | |
|---|---|---|---|---|
| | #2 | #26 A | #26 B | #13 |
| HA-1.1 | 5.10% | 10.57% | 12.63% | 5.19% |
| HA-1.2 | 4.10% | 4.12% | 8.97% | 4.36% |
| HA-2.1 | 3.14% | 12.65% | 11.09% | 5.07% |
| HA-2.2 | 3.65% | 10.82% | 10.04% | 3.81% |
| HB-1.1 | 4.60% | 15.01% | 12.71% | 3.67% |
| HB-1.2 | 4.09% | 11.02% | 10.38% | 5.62% |
| HB-2.1 | 3.37% | 12.80% | 11.12% | 4.24% |

Comparing the performance of these aptamers across different hair samples is difficult due to the high level of variability of the hair. To overcome this constraint, the relative performance of each aptamer for each hair sample is determined by comparing the binding value of the specific aptamer against the average binding value of all aptamers for the respective hair sample (see Table 4).

TABLE 4
Relative performance of the truncated aptamers for different hair samples.

| | Hair sample | | | |
|---|---|---|---|---|
| | #2 | #26 A | #26 B | #13 |
| HA-1.1 | 27% | −4% | 15% | 14% |
| HA-1.2 | 2% | −62% | −18% | −4% |
| HA-2.1 | −22% | 15% | 1% | 11% |
| HA-2.2 | −9% | −2% | −9% | −17% |
| HB-1.1 | 15% | 36% | 16% | −20% |
| HB-1.2 | 2% | 0% | −6% | 23% |
| HB-2.1 | −16% | 16% | 1% | −7% |

It is clear that the truncated aptamer HA-1.1 performed much better than the truncated aptamer HA-1.2 for all the hair samples, indicating that the motif ACAAGT provided higher binding affinity than the motif CGAGCAC.

For truncated aptamers from HA-2, both truncations performed well with HA-2.1 performing better on the damaged hair sample #26. The presence of the structure enabled by this motif is presumed to be responsible for the superior binding properties of this aptamer. The truncated aptamer HB-1.1 performed better than the truncated aptamer HB-1.2. This improvement in performance is correlated with the presence of two conserved motifs in this aptamer versus only one of the conserved motifs in HB-1.2. The binding performance of the truncated aptamer HB-2.1 demonstrates that this portion of the structure is all that is necessary to maintain the binding affinity of the full HB-2 aptamer.

### Example 7. Delivery of a Hair Care Active Ingredient with Aptamers

Aptamers of the current invention are chemically synthesized. A solution of a hair care active ingredient containing a free amine group (0.25 M) and imidazole (0.1 M) in water (pH 6) is prepared. Then, EDC (1-ethyl-3-[3-dimethylaminopropyl]carbodiimide hydrochloride) is weighed in a reaction vial and mixed with an aliquot of an aptamer solution. An aliquot of the amine/imidazole solution is added immediately to the reaction vial and vortexed until all the components are dissolved. An additional aliquot of imidazole solution (0.1 M, pH 6) is added to the reaction vial and the reaction mixture is incubated at room temperature for at least 2 hours. Following incubation, the unreacted EDC and its by-products and imidazole are separated from the modified aptamer by dialysis or by using a spin desalting column and a suitable buffer (e.g. 10 mM sodium phosphate, 0.15 M NaCl, 10 mM EDTA, pH 7.2). Additional details about the conjugation protocols are described in “Hermanson G. T. (2008). Bioconjugate Techniques. 2nd Edition. pp. 969-1002, Academic Press, San Diego.”, the content of which is incorporated herein by reference.

The produced modified aptamer conjugated with a hair care active ingredient can be formulated in hair care composition (e.g. shampoo or conditioner) to provide benefits when contacted with hair.

TABLE 5
List of top sequences from selection
experiments A and B.

| SEQ | | |
|---|---|---|
| ID | | |
| NO | Name | Sequence |
| 1 | H-A1 | AACTACATGGTATGTGGTGAACTGAATATGGATTACAA |
| | | GTTTCAGATCGAGCACTCCCATTCAGACGTACAATGTACCC |
| 2 | H-A2 | AACTACATGGTATGTGGTGAACTAGGGGAACCTTAGTAA |
| | | ACCACGACCCAGGATGTGCTATCGGACGTACAATGTACCC |
| 3 | H-A3 | AACTACATGGTATGTGGTGAACTCAACTTTTAAGCAAGCT |
| | | GTCTACCACGGAGGCAGTATCACGACGTACAATGTACCC |
| 4 | H-A4 | AACTACATGGTATGTGGTGAACTACCGAAATCCAAAAAGC |
| | | AGAACCACCGACCTACAATGGCGGACGTACAATGTACCC |
| 5 | H-A5 | AACTACATGGTATGTGGTGAACTGCCCCGACGAACCAAGG |
| | | AGATCGCAGTTACTACTACCGTAGACGTACAATGTACCC |
| 6 | H-A6 | AACTACATGGTATGTGGTGAACTGCCGAAAGAGGCCATGT |
| | | AAACCACGTATAAGTAGCCCATAGACGTACAATGTACCC |
| 7 | H-A7 | AACTACATGGTATGTGGTGAACTCAGCACGAGAAGTTCGCG |
| | | CCACAGACAGTGCCTAAGCCAGGACGTACAATGTACCC |
| 8 | H-A8 | AACTACATGGTATGTGGTGAACTGAGAATGAAACAGCAGT |
| | | TTTGCGACACGGCCAACGTATTAGACGTACAATGTACCC |
| 9 | H-A9 | AACTACATGGTATGTGGTGAACTCAGACAACCTGCTCAGTT |
| | | AGACCGATTTGACGAGCAACACGACGTACAATGTACCC |
| 10 | H- | AACTACATGGTATGTGGTGAACTTTCGCGGATATTGCTGAT |
| | A10 | ATATTGCCCACAGACGTATGGAGACGTACAATGTACCC |
| 11 | H- | AACTACATGGTATGTGGTGAACTAGTTCCACAAGATGCAG |
| | A11 | AAGCATACACCGCGTCTAGAAGCGACGTACAATGTACCC |
| 12 | H- | AACTACATGGTATGTGGTGAACTTCAAAGTTATAGCACTAT |
| | A12 | CAGACAGCAGAGACCATGACAAGACGTACAATGTACCC |
| 13 | H- | AACTACATGGTATGTGGTGAACTAAGCGGCCCGCAAAACGT |
| | A13 | TTGCGAAGCGGTTCATCTACCAGACGTACAATGTACCC |
| 14 | H- | AACTACATGGTATGTGGTGAACTTCCAGGTCGCGTAGGTC |
| | A14 | TAACGTTCCTGAACAGTTTCATCGACGTACAATGTACCC |
| 15 | H- | AACTACATGGTATGTGGTGAACTAAGACAAATGTCATGC |
| | A15 | ACCATATACAGGGCCAGCCAGCTAGACGTACAATGTACCC |
| 16 | H- | AACTACATGGTATGTGGTGAACTACCAGAGAACATACCCA |
| | A16 | GGCAATTTATATCGCTCTAATGAGACGTACAATGTACCC |
| 17 | H- | AACTACATGGTATGTGGTGAACTGAGCGATGACGAAAAGT |
| | A17 | GTAATGCCAAGACCACGCGGTTAGACGTACAATGTACCC |
| 18 | H- | AACTACATGGTATGTGGTGAACTTACGAAGGCAGCTGCAT |
| | A18 | AAGATACAGAGAGATCCACCACTGACGTACAATGTACCC |
| 19 | H- | AACTACATGGTATGTGGTGAACTTTAATGATTAACGATTA |
| | A19 | ACTTCAATGTTTACCACGCCGAGGACGTACAATGTACCC |
| 20 | H- | AACTACATGGTATGTGGTGAACTGACCTATATCCCTGCGAT |
| | A20 | CTGCAGAGGAATAGTGAAGTTCGACGTACAATGTACCC |
| 21 | H- | AACTACATGGTATGTGGTGAACTACAAAGACCGCATCGAT |
| | A21 | CTATGCCATGGACTAATTCAGCAGACGTACAATGTACCC |
| 22 | H- | AACTACATGGTATGTGGTGAACTGATAGCGGGCTCCAGCAA |
| | A22 | TTACCAAAACTTACCAGCGTCAGACGTACAATGTACCC |
| 23 | H- | AACTACATGGTATGTGGTGAACTCTATCACCCACGTTACT |
| | A23 | ACGTCACTACGAGCAACTCATGAGACGTACAATGTACCC |
| 24 | H- | AACTACATGGTATGTGGTGAACTGCAGCCGATACGCTTA |
| | A24 | GCTGGTTCATATTCACCCCCCAAAGACGTACAATGTACCC |
| 25 | H- | AACTACATGGTATGTGGTGAACTCTGATTTCAGAATCTCG |
| | A25 | GAACCCGCCCGTCATCCATTATGGACGTACAATGTACCC |
| 26 | H- | AACTACATGGTATGTGGTGAACTTCCACACACTGAGAA |
| | A26 | GGCACAAGCAACGCCGTATAGTCATGACGTACAATGTACCC |
| 27 | H- | AACTACATGGTATGTGGTGAACTGCTTTGAACTATAAAGC |
| | A27 | AAATCAGCACGCGTTGCCACGAAGACGTACAATGTACCC |
| 28 | H- | AACTACATGGTATGTGGTGAACTCGTGAGGCGTAACTTA |
| | A28 | ACATGGAGCCTCTACTGATCCACAGACGTACAATGTACCC |
| 29 | H- | AACTACATGGTATGTGGTGAACTAGCATATGATTTGCAGC |
| | A29 | ATCATATATAAAACTGTTCCCCAGACGTACAATGTACCC |
| 30 | H- | AACTACATGGTATGTGGTGAACTGGAGCACTTTAGGGTGA |
| | A30 | TAGTGACAGACCACCGTACCACAGACGTACAATGTACCC |
| 31 | H- | AACTACATGGTATGTGGTGAACTTGACCTAATCATCCAAA |
| | A31 | TGGAGTTTTACAGAAACTGCGAGGACGTACAATGTACCC |
| 32 | H- | AACTACATGGTATGTGGTGAACTGGAGCGTGACAAACACT |
| | A32 | GGTCCGACGCAGCACACTCACCTGACGTACAATGTACCC |
| 33 | H- | AACTACATGGTATGTGGTGAACTCGAGGCGTCATTAGCCC |
| | A33 | ACAGCATGGCACATACTAAGAGAGACGTACAATGTACCC |
| 34 | H- | AACTACATGGTATGTGGTGAACTCAACCAGAAACCTAGA |
| | A34 | GGTAAATAGGAATTGAGGGAGACAGACGTACAATGTACCC |
| 35 | H- | AACTACATGGTATGTGGTGAACTCGCGCATTCTTGAACAG |
| | A35 | ATAATACTCGGCGCAAGATACCGGACGTACAATGTACCC |
| 36 | H- | AACTACATGGTATGTGGTGAACTACTTTGACGGTGCCAAG |
| | A36 | AGAACTAGCTTAAGTCCGTGTTCGACGTACAATGTACCC |
| 37 | H- | AACTACATGGTATGTGGTGAACTAATAACAAGGTGCCAA |
| | A37 | AAACCTCTCAGAAACAAGAACCCCGACGTACAATGTACCC |
| 38 | H- | AACTACATGGTATGTGGTGAACTTCAACGCGGGAGTCGAC |
| | A38 | AACCAACTACCAAACTGCGGAGAGACGTACAATGTACCC |
| 39 | H- | AACTACATGGTATGTGGTGAACTTAATGAGCGCACATATA |
| | A39 | CAAGTAAGTAGCAGCGAGAATCAGACGTACAATGTACCC |
| 40 | H- | AACTACATGGTATGTGGTGAACTCACAGACATTAGAATGT |
| | A40 | GACTCGCCGCAAACCGATAGACAGACGTACAATGTACCC |
| 41 | H- | AACTACATGGTATGTGGTGAACTGGACAACGTTTAAATGT |
| | A41 | GCCGAAACCGCATAGACGTATTGGACGTACAATGTACCC |
| 42 | H- | AACTACATGGTATGTGGTGAACTCGGACAAAGAGCTCAAT |
| | A42 | CCTGGACAGCACGTAGGTATGTAGACGTACAATGTACCC |
| 43 | H- | AACTACATGGTATGTGGTGAACTAGGTATCGCCAGACTATA |
| | A43 | TAGTAAGTCGAACAGAACCACCGACGTACAATGTACCC |
| 44 | H- | AACTACATGGTATGTGGTGAACTCCTCGACTGTCATCGCAT |
| | A44 | CCAAGCGTGCACCAGAAGCTCAGACGTACAATGTACCC |
| 45 | H- | AACTACATGGTATGTGGTGAACTACTGTTTATGTGCCGATGT |
| | A45 | ATAAGCAAGTATTCGATCACCGACGTACAATGTACCC |
| 46 | H- | AACTACATGGTATGTGGTGAACTGTGTTGAACTGATCATGGC |
| | A46 | CCTGATCGCTCAACGGCTCAAGACGTACAATGTACCC |
| 47 | H- | AACTACATGGTATGTGGTGAACTAAGGCGCTATCGGGAAC |
| | A47 | GCAGCCCTTTCTACCAAACCCAAGACGTACAATGTACCC |
| 48 | H- | AACTACATGGTATGTGGTGAACTCGAGCAGAAGGTCCACCG |
| | A48 | GCAACGCAATTACCAAGAATCCGACGTACAATGTACCC |
| 49 | H- | AACTACATGGTATGTGGTGAACTTTAGTTTAGATCAACACA |
| | A49 | CCCTGATTGCAACTGCTGCATAGACGTACAATGTACCC |
| 50 | H- | AACTACATGGTATGTGGTGAACTAAAGAGGCAGACGCGTA |
| | A50 | ATCATAGCAGCCAAAATAGACACGACGTACAATGTACCC |
| 51 | H- | AACTACATGGTATGTGGTGAACTGTAAGTCCCACAAATGCA |
| | A51 | TTCAGGCTAGCTCATGTAGCACGACGTACAATGTACCC |
| 52 | H- | AACTACATGGTATGTGGTGAACTTTCATTGCCTGCGTAAAC |
| | A52 | CACACGGTCCGTTATAAACTTAGACGTACAATGTACCC |
| 53 | H- | AACTACATGGTATGTGGTGAACTTGACCAAACCAGCCT |
| | A53 | ATGAGTGATAAGCTTCTGTGCAGTAGACGTACAATGTACCC |
| 54 | H- | AACTACATGGTATGTGGTGAACTGGCTGAGACGAACCA |
| | A54 | CTAGGGTGATCACCAAACCCGCTCAGACGTACAATGTACCC |
| 55 | H- | AACTACATGGTATGTGGTGAACTTGACAAATAAGGATAG |
| | A55 | AATCAACATCACAAGCAGGCAGTTGACGTACAATGTACCC |
| 56 | H- | AACTACATGGTATGTGGTGAACTTAAATTTGCCACAATAT |
| | A56 | CTTGGCCCCATAGAAGGGCCGTCGACGTACAATGTACCC |
| 57 | H- | AACTACATGGTATGTGGTGAACTAATAACACATAACACAC |
| | A57 | GCGAACCAATCTCCCGGCCCAAAGACGTACAATGTACCC |
| 58 | H- | AACTACATGGTATGTGGTGAACTTGATGCCAATGACAACG |
| | A58 | CCACACGTTCGACACACATACACGACGTACAATGTACCC |
| 59 | H- | AACTACATGGTATGTGGTGAACTAAAACGGGTTTAGATCA |
| | A59 | TCACGAGGACTCATGCGGGATTTGACGTACAATGTACCC |
| 60 | H- | AACTACATGGTATGTGGTGAACTGAAATCGCCACAGAGTC |
| | A60 | TTTGCGGAAGAGCGTGAAAAGCAGACGTACAATGTACCC |
| 61 | H- | AACTACATGGTATGTGGTGAACTCCCCGATCTCCATCGAT |
| | A61 | CTTCAAGATAGGAAAGGACACCAGACGTACAATGTACCC |
| 62 | H- | AACTACATGGTATGTGGTGAACTGTCTCGAGGTTCATAAG |
| | A62 | CTATGGAAACAAGCACCGCATATGACGTACAATGTACCC |
| 63 | H- | AACTACATGGTATGTGGTGAACTACCGTCAAATGGTGACT |
| | A63 | TTCGAGTTTGCCACACCTAAGAGGACGTACAATGTACCC |
| 64 | H- | AACTACATGGTATGTGGTGAACTGTCCCGCAATCCAAAAT |
| | A64 | CGCGCACAAGAGCCCACAGCCAGGACGTACAATGTACCC |
| 65 | H- | AACTACATGGTATGTGGTGAACTGGCCCCGTCTAGGACGA |
| | A65 | CCAACACCTGCCGTCGACTGTGAGACGTACAATGTACCC |
| 66 | H- | AACTACATGGTATGTGGTGAACTCGTCTGAGCCACCTTAA |
| | A66 | CCAGATTTGATAACCCACAGCGAGACGTACAATGTACCC |
| 67 | H- | AACTACATGGTATGTGGTGAACTATGTGAATTCAAGGAAT |
| | A67 | TGCAGCCACATAGCGCCGAATACGACGTACAATGTACCC |
| 68 | H- | AACTACATGGTATGTGGTGAACTGGAGGACGTCGTAAGAT |
| | A68 | GTTACAAAGGCACTCCCGAACTAGACGTACAATGTACCC |
| 69 | H- | AACTACATGGTATGTGGTGAACTGCCATTGACAGAGAGGA |
| | A69 | GAAATCTTTTGAGCAGTGAGCACGACGTACAATGTACCC |
| 70 | H- | AACTACATGGTATGTGGTGAACTAACTTTGCGGCACCCAC |
| | A70 | AAGAGTTCGTAAAAGCAGACACCGACGTACAATGTACCC |
| 71 | H- | AACTACATGGTATGTGGTGAACTTGTGGCGGCGAACACAC |
| | A71 | CATGAGCACCTCACATGACCGTGGACGTACAATGTACCC |
| 72 | H- | AACTACATGGTATGTGGTGAACTCAACGAACAGTAGCTATG |
| | A72 | ATAACAGCCTTCGACGTGTCCAGACGTACAATGTACCC |
| 73 | H- | AACTACATGGTATGTGGTGAACTGCCCTTACGGCACATAC |
| | A73 | AGTGACTCATGGCGGCAGCTAACGACGTACAATGTACCC |
| 74 | H- | AACTACATGGTATGTGGTGAACTGTAGCATTGCCGAGAGC |
| | A74 | TCACCTGTTTTACACGCGAGTTAGACGTACAATGTACCC |
| 75 | H- | AACTACATGGTATGTGGTGAACTGATCCGTAGGTCACAC |
| | A75 | CTTTATGCCATCCGGGACCAATTCGACGTACAATGTACCC |
| 76 | H- | AACTACATGGTATGTGGTGAACTCGATCTGTACGAGACT |
| | A76 | CGATCCTACGCACAGCACCCCAGTGACGTACAATGTACCC |
| 77 | H- | AACTACATGGTATGTGGTGAACTTCCTACAAAGCTATTTG |
| | A77 | CAGGTCGGACGTGGATACCAATTGACGTACAATGTACCC |
| 78 | H- | AACTACATGGTATGTGGTGAACTAGGCAAAAACAACCTT |
| | A78 | AACCTTGAGCCCACAAGCCAGATAGACGTACAATGTACCC |
| 79 | H- | AACTACATGGTATGTGGTGAACTTCCGAAATGAAAAAAG |
| | A79 | TTACCCGACACGGCCAAGGCTAGTGACGTACAATGTACCC |
| 80 | H- | AACTACATGGTATGTGGTGAACTAGAACGGAGAAGTCC |
| | A80 | GGTCCGAGTATCTTTAAATACCAGCGACGTACAATGTACCC |
| 81 | H- | AACTACATGGTATGTGGTGAACTCTGAATGCGAGATGTA |
| | A81 | CAACACGGATCGACGTAGCTTATTGACGTACAATGTACCC |
| 82 | H- | AACTACATGGTATGTGGTGAACTGCAGTACAAAATGCGG |
| | A82 | TTTCTTTCACAACGATTAGTAGTCGACGTACAATGTACCC |
| 83 | H- | AACTACATGGTATGTGGTGAACTAGGAACTACAACGTTG |
| | A83 | GTCCTGAAATCACAACCATCTAAAGACGTACAATGTACCC |
| 84 | H- | AACTACATGGTATGTGGTGAACTTCCAAACCAAATTAGG |
| | A84 | ATGATCCAGCTCGCCACAGCCAAGGACGTACAATGTACCC |
| 85 | H- | AACTACATGGTATGTGGTGAACTCGGAAGAAGGAGGCCAC |
| | A85 | ATCCTGGAGCAACAAGACGAGAAGACGTACAATGTACCC |
| 86 | H- | AACTACATGGTATGTGGTGAACTATGCTACACGGAGACC |
| | A86 | GAAGCTCTTACGAGATAGTTCTCAGACGTACAATGTACCC |
| 87 | H- | AACTACATGGTATGTGGTGAACTAGAGGCGGCTTAACCCT |
| | A87 | ACAGCTACCCCGACATCAAGTCCGACGTACAATGTACCC |
| 88 | H- | AACTACATGGTATGTGGTGAACTTGGATAGTGTGGCTGA |
| | A88 | AATACCAATTAACCAAACCAATGCGACGTACAATGTACCC |
| 89 | H- | AACTACATGGTATGTGGTGAACTAACAAAACCGAATCTG |
| | A89 | TGGAGCGCCACAACCCAAATACTAGACGTACAATGTACCC |
| 90 | H- | AACTACATGGTATGTGGTGAACTGGGAAACAGAAGACCA |
| | A90 | CATTACTCAATGCGAATATCGACTGACGTACAATGTACCC |
| 91 | H- | AACTACATGGTATGTGGTGAACTATGGAAAAAAGGATGGT |
| | A91 | CCCACCTCCCAAAACCATTGTCAGACGTACAATGTACCC |
| 92 | H- | AACTACATGGTATGTGGTGAACTATAGAAACTGACCACCA |
| | A92 | GTCACACCCTGAGAAGAAGCAGAGACGTACAATGTACCC |
| 93 | H- | AACTACATGGTATGTGGTGAACTAACTGACATGGTCTCTG |
| | A93 | AGACGGCCATAGAGTGTTCAAAAGACGTACAATGTACCC |
| 94 | H- | AACTACATGGTATGTGGTGAACTCTAATAAACGGCGGGCT |
| | A94 | GAATTAGAGACGACACAACCGCAGACGTACAATGTACCC |
| 95 | H- | AACTACATGGTATGTGGTGAACTTAAGCGGCCCTTAGGAG |
| | A95 | CGTTGGTACCACATTCATGGAGAGACGTACAATGTACCC |
| 96 | H- | AACTACATGGTATGTGGTGAACTGCAGAGAGGCGGTTAGC |
| | A96 | CCAGAAATCAACCACGTGCCATAGACGTACAATGTACCC |
| 97 | H- | AACTACATGGTATGTGGTGAACTGTGATGACGAAGGTCAT |
| | A97 | AGGTCAGCCAACATGCCTGTGAGGACGTACAATGTACCC |
| 98 | H- | AACTACATGGTATGTGGTGAACTTTCCGTATATCGGACCG |
| | A98 | GTAAGTCTACCTAACATACGTGAGACGTACAATGTACCC |
| 99 | H- | AACTACATGGTATGTGGTGAACTAGTCGGCAAAGGAGGA |
| | A99 | TCCACAACATAACGAGAGTAACTGGACGTACAATGTACCC |
| 100 | H- | AACTACATGGTATGTGGTGAACTTAAAGTTACCCTGAGCA |
| | A100 | ATGCAGCGACGAAATAACGTTGAGACGTACAATGTACCC |
| 101 | H-B1 | AACTACATGGTATGTGGTGAACTTAGTGGGATTTATTCAC |
| | | TATGTACACCGATGACAACAGTAGACGTACAATGTACCC |
| 102 | H-B2 | AACTACATGGTATGTGGTGAACTGCAGAACATGACCAAAA |
| | | GAGGAAAGGTATAGCTGCTATCAGACGTACAATGTACCC |
| 103 | H-B3 | AACTACATGGTATGTGGTGAACTTAGTCACGATATCGTGGC |
| | | CCAGAACCTCAATCATGCAAAAGACGTACAATGTACCC |
| 104 | H-B4 | AACTACATGGTATGTGGTGAACTTCAGCGGTGAACACATC |
| | | CAATCAAGAAGGCCACTATACGAGACGTACAATGTACCC |
| 105 | H-B5 | AACTACATGGTATGTGGTGAACTCTGAATGATGAACTGTA |
| | | TCCGAACACCAAACCAAATCCCTGACGTACAATGTACCC |
| 106 | H-B6 | AACTACATGGTATGTGGTGAACTGCTCGATAGACAGGCCT |
| | | AAAACCCCCGGACGAACCTTTCAGACGTACAATGTACCC |
| 107 | H-B7 | AACTACATGGTATGTGGTGAACTAAATATTTAGAATTC |
| | | TGGTTCACGACAACATGAACAGGTGGACGTACAATGTACCC |
| 108 | H-B8 | AACTACATGGTATGTGGTGAACTAAGGAGAACGAAGTGCAC |
| | | TTGCAACTTCACTATCAGCACAGACGTACAATGTACCC |
| 109 | H-B9 | AACTACATGGTATGTGGTGAACTGAATCCGAACACAAGAA |
| | | CATGACGGAAGGCTTATACCGATGACGTACAATGTACCC |
| 110 | H- | AACTACATGGTATGTGGTGAACTGCCAGGGACCTTCAACC |
| | B10 | GATGAGGTGACAGACTGACAATTGACGTACAATGTACCC |
| 111 | H- | AACTACATGGTATGTGGTGAACTATCGATGGATCTCCAA |
| | B11 | TCGACAGTCACTCTGAACCCTTTAGACGTACAATGTACCC |
| 112 | H- | AACTACATGGTATGTGGTGAACTACGAAGGGAACTGCT |
| | B12 | CACCAACAACACGCCCGTAGGACTCGACGTACAATGTACCC |
| 113 | H- | AACTACATGGTATGTGGTGAACTAGATGAAGACACCGAC |
| | B13 | TTAAGCCGACGTAATCTTCTAGAAGACGTACAATGTACCC |
| 114 | H- | AACTACATGGTATGTGGTGAACTACATTATGATAAGCCGAG |
| | B14 | TCCACGTGCTTCATACAATCTAGACGTACAATGTACCC |
| 115 | H- | AACTACATGGTATGTGGTGAACTTGTAAAAGTTGAGGA |
| | B15 | CATACCAACGCTAAAGAACGAGCTAGACGTACAATGTACCC |
| 116 | H- | AACTACATGGTATGTGGTGAACTATCCCCTCGACTCCA |
| | B16 | GCGTTTCAGAATCGCTTACCAGTAGGACGTACAATGTACCC |
| 117 | H- | AACTACATGGTATGTGGTGAACTATCGGAGCGACGACGCG |
| | B17 | CTAATAAGCCCACTATGGATGTAGACGTACAATGTACCC |
| 118 | H- | AACTACATGGTATGTGGTGAACTGGATAACTGGATCACCG |
| | B18 | ACTTTGAAACGCTCCATGTGGATGACGTACAATGTACCC |
| 119 | H- | AACTACATGGTATGTGGTGAACTACCAAAAAGCAGAGCCT |
| | B19 | GGCACAGCGCTACAAGGCAGATAGACGTACAATGTACCC |
| 120 | H- | AACTACATGGTATGTGGTGAACTTGCACTATGACAACCTC |
| | B20 | TAGACTGCTGCATTTGAAACCACGACGTACAATGTACCC |
| 121 | H- | AACTACATGGTATGTGGTGAACTAGTTAGACCACTCACAG |
| | B21 | TCCATTAAGGCAGCTAGGAGCCAGACGTACAATGTACCC |
| 122 | H- | AACTACATGGTATGTGGTGAACTTGAGCAGAGACGTTCA |
| | B22 | GCGAAGGTCTCCGCCTTCGAATCCGACGTACAATGTACCC |
| 123 | H- | AACTACATGGTATGTGGTGAACTCCTCTGAGCATAAGTCG |
| | B23 | AGGAAAAACCGCCGACCAATATAGACGTACAATGTACCC |
| 124 | H- | AACTACATGGTATGTGGTGAACTTCCAAATGGACACACCCG |
| | B24 | CATAGACCGAGTTGTACCTGAAGACGTACAATGTACCC |
| 125 | H- | AACTACATGGTATGTGGTGAACTATGAGAGAACACGGGCA |
| | B25 | TACTTGCATCCCATATACGTTTAGACGTACAATGTACCC |
| 126 | H- | AACTACATGGTATGTGGTGAACTTATCGCCGTAAGATTCT |
| | B26 | GACAAACCCACGGAATCACCCAAGACGTACAATGTACCC |
| 127 | H- | AACTACATGGTATGTGGTGAACTCGAGAAGCAGCGCCAT |
| | B27 | ACCTACTGACGGACACATACGAAGGACGTACAATGTACCC |
| 128 | H- | AACTACATGGTATGTGGTGAACTTCAGACTTAAGGATACG |
| | B28 | CTGAGCCAACACCACATCATCGAGACGTACAATGTACCC |
| 129 | H- | AACTACATGGTATGTGGTGAACTCGGATTTCGCAGAGGA |
| | B29 | ATTGAGCTGCAGATCCCGAGCAGAGACGTACAATGTACCC |
| 130 | H- | AACTACATGGTATGTGGTGAACTCAATGAGCTCGAAACGC |
| | B30 | GGAAATCCATGCCATGGAAGACGGACGTACAATGTACCC |
| 131 | H- | AACTACATGGTATGTGGTGAACTAATTCCACGGAAAGAG |
| | B31 | TTAAGCAGCCCGCGTTACATGAGTGACGTACAATGTACCC |
| 132 | H- | AACTACATGGTATGTGGTGAACTATTACGACAGAACTGT |
| | B32 | TGCCCAGTCTCCAGCGCGCTCACGGACGTACAATGTACCC |
| 133 | H- | AACTACATGGTATGTGGTGAACTGAGGCGAAAAAGCGC |
| | B33 | ACAATTAAGACCACAAGTCAGTGCAGACGTACAATGTACCC |
| 134 | H- | AACTACATGGTATGTGGTGAACTAAAATAGTTTGGTCTTA |
| | B34 | TCCCAAATACGCAAAGTGTCTTGGACGTACAATGTACCC |
| 135 | H- | AACTACATGGTATGTGGTGAACTTTAAGTAGTCACGTTAG |
| | B35 | AGTCCACGGCACCCGCATACAATGACGTACAATGTACCC |
| 136 | H- | AACTACATGGTATGTGGTGAACTTGCTAGGTTAGGAAGA |
| | B36 | AAGACATTTTTAGTCACCACACAGGACGTACAATGTACCC |
| 137 | H- | AACTACATGGTATGTGGTGAACTCAGCTAGCTCCGCCAG |
| | B37 | AACAGTAACCACCACATCAGCAGAGACGTACAATGTACCC |
| 138 | H- | AACTACATGGTATGTGGTGAACTTGACACAAATCATGGGA |
| | B38 | TGGAATCATAAAGGTTGTTCACAGACGTACAATGTACCC |
| 139 | H- | AACTACATGGTATGTGGTGAACTCGGCCTTACGGAGGAAG |
| | B39 | GGAAGTACATCCACTACCGAGTTGACGTACAATGTACCC |
| 140 | H- | AACTACATGGTATGTGGTGAACTCGAATTTGACCTGCATT |
| | B40 | GGATTCTGGTCCTTTTGCCACAAGACGTACAATGTACCC |
| 141 | H- | AACTACATGGTATGTGGTGAACTTCAACGCTATAGAGTGT |
| | B41 | TATAGTCAACGAACACATACGCAGACGTACAATGTACCC |
| 142 | H- | AACTACATGGTATGTGGTGAACTAAGAGATATATTTCCCA |
| | B42 | AGTCCCACAGAACCCCGATAGAGGACGTACAATGTACCC |
| 143 | H- | AACTACATGGTATGTGGTGAACTGACGCGAGTGCCCAATG |
| | B43 | CATAAAGGGAGCGCCCTAACCGTGACGTACAATGTACCC |
| 144 | H- | AACTACATGGTATGTGGTGAACTGTAAGCAAACCTCCATC |
| | B44 | CGCGATAAATAAGCTCGCCCCATGACGTACAATGTACCC |
| 145 | H- | AACTACATGGTATGTGGTGAACTCAGATAAGTTCCGTACA |
| | B45 | TACAGGGCCACAGAGGCAAGATAGACGTACAATGTACCC |
| 146 | H- | AACTACATGGTATGTGGTGAACTTAAGTCAGCATCATACA |
| | B46 | GTCATGGATGTGCCAAGTCAGATGACGTACAATGTACCC |
| 147 | H- | AACTACATGGTATGTGGTGAACTACTAGGACACGAAGAC |
| | B47 | GCACAGCGATCCTAAAGAGCCAACGACGTACAATGTACCC |
| 148 | H- | AACTACATGGTATGTGGTGAACTTGGCGGAAACCAACCTT |
| | B48 | GAGCACTGTACCATGTTCGAGCAGACGTACAATGTACCC |
| 149 | H- | AACTACATGGTATGTGGTGAACTCAAGGCGATAAGACCAT |
| | B49 | ATAAATGGAATCACATTAAGATCGACGTACAATGTACCC |
| 150 | H- | AACTACATGGTATGTGGTGAACTGGCCCTGTATAAAGATT |
| | B50 | CGACTCTGTCAACCACAAACCTAGACGTACAATGTACCC |
| 151 | H- | AACTACATGGTATGTGGTGAACTTAGGCTCAATACTTAC |
| | B51 | CTGATGACAGGCGCCCGCATCACAGACGTACAATGTACCC |
| 152 | H- | AACTACATGGTATGTGGTGAACTCGGAAGAGCTACTCAC |
| | B52 | ACCGCCAAGGACCATAAGTTCTTTGACGTACAATGTACCC |
| 153 | H- | AACTACATGGTATGTGGTGAACTGAACACCTATTGACAT |
| | B53 | GCCAACAGTGGCGGACCATTAGTTGACGTACAATGTACCC |
| 154 | H- | AACTACATGGTATGTGGTGAACTGTCCGAAAAGACGATC |
| | B54 | AGACGACCATATGTTAACTGAGCTGACGTACAATGTACCC |
| 155 | H- | AACTACATGGTATGTGGTGAACTCTGCTCAAATAAACCCA |
| | B55 | TCAACTGAGAAAGCCAAATGTTCGACGTACAATGTACCC |
| 156 | H- | AACTACATGGTATGTGGTGAACTTCGGGTTGAGACCACG |
| | B56 | TCCATGCATTGCGCACGGTTCAGTGACGTACAATGTACCC |
| 157 | H- | AACTACATGGTATGTGGTGAACTAAGGCGGGAGATCCTTG |
| | B57 | TTAACAGGCCACCCAACCGAGTAGACGTACAATGTACCC |
| 158 | H- | AACTACATGGTATGTGGTGAACTTCCACATGATCCGACT |
| | B58 | TCAGCCGAGCGTTCCTACGCAGCAGACGTACAATGTACCC |
| 159 | H- | AACTACATGGTATGTGGTGAACTATCCAAGGAATCGAAAA |
| | B59 | CCTGTCTCCACGTGGGCATCTCTGACGTACAATGTACCC |
| 160 | H- | AACTACATGGTATGTGGTGAACTGTCCATTCTTGACCACT |
| | B60 | AACAATCCCACCAGGCGAGGTGTGACGTACAATGTACCC |
| 161 | H- | AACTACATGGTATGTGGTGAACTCAACCGATCCGCGAC |
| | B61 | TCAACCGATAAATAAGCCATCCACAGACGTACAATGTACCC |
| 162 | H- | AACTACATGGTATGTGGTGAACTTGTCTATTTGTTCCCAAC |
| | B62 | TAAACGTCAGCAACACACCAACGACGTACAATGTACCC |
| 163 | H- | AACTACATGGTATGTGGTGAACTAAGAACAGAATGTCTGA |
| | B63 | TCCCTGGCGAGACCAATATCCATGACGTACAATGTACCC |
| 164 | H- | AACTACATGGTATGTGGTGAACTTAACCAACGCCACACTG |
| | B64 | ACATGCGCCATTATCAAGGAGTAGACGTACAATGTACCC |
| 165 | H- | AACTACATGGTATGTGGTGAACTGTCTGATGATCTGGTCT |
| | B65 | CGATTCAGTAGATAACAGCCACCGACGTACAATGTACCC |
| 166 | H- | AACTACATGGTATGTGGTGAACTCGGCACAGAACTACCCT |
| | B66 | CCAACAAGAGAGCGCCTTTATCAGACGTACAATGTACCC |
| 167 | H- | AACTACATGGTATGTGGTGAACTCCCACTCGCTCAGTCGG |
| | B67 | GAAGACCGGTGGTAGGAGCCTTAGACGTACAATGTACCC |
| 168 | H- | AACTACATGGTATGTGGTGAACTGGATTGGGATATCAGAA |
| | B68 | TTTAATCAGCTCACAAGCAAACCGACGTACAATGTACCC |
| 169 | H- | AACTACATGGTATGTGGTGAACTCAGTCAGTCTAAGGTAA |
| | B69 | CACAACTTGCATGGATGAACACCGACGTACAATGTACCC |
| 170 | H- | AACTACATGGTATGTGGTGAACTGCAGCTAACCACTGAAC |
| | B70 | TGGTCGTAGCCCGCAACAACAGAGACGTACAATGTACCC |
| 171 | H- | AACTACATGGTATGTGGTGAACTCGAGTAAGTCAAACGCT |
| | B71 | CACCATCTTACAAGGCGCATCTAGACGTACAATGTACCC |
| 172 | H- | AACTACATGGTATGTGGTGAACTGCTCATACACTGCAAGG |
| | B72 | AAGTAGAGCGGTGTAACAGTCCCGACGTACAATGTACCC |
| 173 | H- | AACTACATGGTATGTGGTGAACTAGGCGCCACATGGCAAT |
| | B73 | AACGGTCCGCTATAGTCGTATTAGACGTACAATGTACCC |
| 174 | H- | AACTACATGGTATGTGGTGAACTCGGAAGGAACCAAGTT |
| | B74 | AATCTTTGAACTGGTCCGAGACTTGACGTACAATGTACCC |
| 175 | H- | AACTACATGGTATGTGGTGAACTGCTCGTATACAAACTAT |
| | B75 | CCTTGTCCGCCACTTGTTGCACCGACGTACAATGTACCC |
| 176 | H- | AACTACATGGTATGTGGTGAACTATCGGTTGTTTACCACG |
| | B76 | GAAACTGCGCAGTTTCGAAAGGCGACGTACAATGTACCC |
| 177 | H- | AACTACATGGTATGTGGTGAACTACTCAACAATTCAGAC |
| | B77 | AGCACGTGTTAAGTATATTGCATAGACGTACAATGTACCC |
| 178 | H- | AACTACATGGTATGTGGTGAACTAAGACAGGCGAACCTGA |
| | B78 | AGTCAAGCAACCACATGCCCGAGGACGTACAATGTACCC |
| 179 | H- | AACTACATGGTATGTGGTGAACTAGGATCAATGTCCTGAAG |
| | B79 | CCAGTCGTTGGCCGTGAATCAAGACGTACAATGTACCC |
| 180 | H- | AACTACATGGTATGTGGTGAACTTGGCGTAAAAGTTAGAA |
| | B80 | CCATCATTGCTCCACGCTACATGGACGTACAATGTACCC |
| 181 | H- | AACTACATGGTATGTGGTGAACTTGCCATTCACGTACCG |
| | B81 | TTAGGGCCGTCCAAATCCACGTAGGACGTACAATGTACCC |
| 182 | H- | AACTACATGGTATGTGGTGAACTAAACGCACATACGATCC |
| | B82 | TGCGCCGAAGATCAAGGTAAGGAGACGTACAATGTACCC |
| 183 | H- | AACTACATGGTATGTGGTGAACTGTTCCAAACACACAACA |
| | B83 | TGGCGTCATGTCACAATTCAATTGACGTACAATGTACCC |
| 184 | H- | AACTACATGGTATGTGGTGAACTGTTACTTGGTAGAGCCA |
| | B84 | AGGCTTTACAAAGTTCGAACTCAGACGTACAATGTACCC |
| 185 | H- | AACTACATGGTATGTGGTGAACTGTATAACGAAATCCAG |
| | B85 | CCACGTACTGCGATACGCGAAAATGACGTACAATGTACCC |
| 186 | H- | AACTACATGGTATGTGGTGAACTCTCTCAGTGAAGCCTGG |
| | B86 | AATAGAATACCACGCACGCGGTCGACGTACAATGTACCC |
| 187 | H- | AACTACATGGTATGTGGTGAACTCAACGAGAGTGGGAGC |
| | B87 | ACCTACAGACGCATGGGCAAATGAGACGTACAATGTACCC |
| 188 | H- | AACTACATGGTATGTGGTGAACTTAAAGGCATAGGACATG |
| | B88 | CTCAGGAGGTCACCGCCAAACCAGACGTACAATGTACCC |
| 189 | H- | AACTACATGGTATGTGGTGAACTACTCGAAGCGTTCCAAT |
| | B89 | TTTGGAGTCTTCTGACACCAGCCGACGTACAATGTACCC |
| 190 | H- | AACTACATGGTATGTGGTGAACTCAGAGTAAAGTCTCG |
| | B90 | CAAGTGCACCGCTAATCTACCCGCAGACGTACAATGTACCC |
| 191 | H- | AACTACATGGTATGTGGTGAACTCGAAAATTCATCCCAC |
| | B91 | AGGCTGGTGGCACGACTAGAACGAGACGTACAATGTACCC |
| 192 | H- | AACTACATGGTATGTGGTGAACTTTCCAAACAATTCAGAG |
| | B92 | ATGGACCACATAAACCCCAATGCGACGTACAATGTACCC |
| 193 | H- | AACTACATGGTATGTGGTGAACTATCATCACACCGTGGA |
| | B93 | AGGATTGAGTCCGACGGAGATCACGACGTACAATGTACCC |
| 194 | H- | AACTACATGGTATGTGGTGAACTTTCCATCTATAACTGTC |
| | B94 | AAAAGCACACCTCGACTACCCGAGACGTACAATGTACCC |
| 195 | H- | AACTACATGGTATGTGGTGAACTACATGGCGAGACGATG |
| | B95 | ATGAGTGCACCAGATCCATTAGATGACGTACAATGTACCC |
| 196 | H- | AACTACATGGTATGTGGTGAACTAGAGTCTAAGAATAGG |
| | B96 | TTAAACCTGGTCAAGCTCAGCCCAGACGTACAATGTACCC |
| 197 | H- | AACTACATGGTATGTGGTGAACTAGCCAAATCCTTCCCTG |
| | B97 | TCGCCAGAGTGATTGGTTCCCAAGACGTACAATGTACCC |
| 198 | H- | AACTACATGGTATGTGGTGAACTAAGCACGGATAATGCG |
| | B98 | TCAAAGTGAGGACAAGCCAAGAATGACGTACAATGTACCC |
| 199 | H- | AACTACATGGTATGTGGTGAACTGCAAAGTATTTCCAAG |
| | B99 | CACCGTAGTAGGGAATCAATGTGAGACGTACAATGTACCC |
| 200 | H- | AACTACATGGTATGTGGTGAACTTGCCATTAATAGCGCGG |
| | B100 | CTAGAACACATTTCACACACAACGACGTACAATGTACCC |

TABLE 6
List of exemplary truncated aptamers.

| SEQ | | |
|---|---|---|
| ID | | |
| NO | Name | Sequence |
| 214 | H-A1.1 | AAAGTGAACTGAATATGGATTACAAGTTTCAGATCGAAA |
| 215 | H-A1.2 | AAACTACATGGTATGTGGTGAACCAAAGGATCGAGCAC |
| | | TCCCATTCAGAAA |
| 216 | H-A2.1 | CTACATGGTATGTGGAAACCACGACCCAGGATGTGC |
| 217 | H-A2.2 | AAAGTGGTGAACTAGGGGAACCTTAGTAAACCACAAA |
| 218 | H-B1.1 | AAAGGTGAACTTAGTGGGATTTATTCACTATGTACACCGAT |
| 219 | H-B1.2 | AATTAGTGGGATTTATTCACTATAA |
| 220 | H-B2.1 | ACTGCAGAACATGACCAAAAGAGGAAAGGTATAGCTGCTA |

### ADDITIONAL EXAMPLES/COMBINATIONS

A. An aptamer composition comprising at least one oligonucleotide consisting of: deoxyribonucleotides, ribonucleotides, derivatives of deoxyribonucleotides, derivatives of ribonucleotides, and mixtures thereof; wherein said aptamer composition has a binding affinity for a material selected from the group consisting of: undamaged hair, damaged hair, hair cuticle, hair epicuticle, hair exocuticle, hair endocuticle, hair cortex, hair keratins, hair F-layer, hair lipids, 18-methyleicosanoic acid, and mixtures thereof.
B. The aptamer composition according to Paragraph A, wherein said aptamer composition has a binding affinity for damaged hair.
C. The aptamer composition according to Paragraph A-B, wherein said aptamer composition has a higher binding affinity for damaged hair than for undamaged hair.
D. The aptamer composition according to Paragraph A-C, comprising at least one oligonucleotide selected from the group consisting of oligonucleotides with at least 50% nucleotide sequence identity to sequences selected from the group consisting of SEQ ID NO 1 to SEQ ID NO 200 and SEQ ID NO 214 to SEQ ID NO 220.
E. The aptamer composition according to Paragraph A-D, comprising at least one oligonucleotide selected from the group consisting of SEQ ID NO 1 to SEQ ID NO 200 and SEQ ID NO 214 to SEQ ID NO 220.
F. The aptamer composition according to Paragraph A-E, comprising at least one oligonucleotide selected from the group consisting of SEQ ID NO 1, SEQ ID NO 2, SEQ ID NO 101, SEQ ID NO 102, and SEQ ID NO 214 to SEQ ID NO 220.
G. The aptamer composition according to Paragraph A-F, wherein said at least one oligonucleotide comprises one or more motifs selected from the group consisting of SEQ ID NO 201 to SEQ ID NO 213.
H. The aptamer composition according to Paragraph A-G, wherein said at least one oligonucleotide comprises natural or non-natural nucleobases.
I. The aptamer composition according to Paragraph A-H, wherein said non-natural nucleobases are selected from the group comprising hypoxanthine, xanthine, 7-methylguanine, 5,6-dihydrouracil, 5-5-methylcytosine, 5-hydroxymethylcytosine, thiouracil, 1-methylhypoxanthine, 6-methylisoquinoline-1-thione-2-yl, 3-methoxy-2-naphthyl, 5-propynyluracil-1-yl, 5-methylcytosin-1-yl, 2-aminoadenin-9-yl, 7-deaza-7-iodoadenin-9-yl, 7-deaza-7-propynyl-2-aminoadenin-9-yl, phenoxazinyl, phenoxazinyl-G-clam, and mixtures thereof.
J. The aptamer composition according to Paragraph A-I, wherein the nucleosides of said at least one oligonucleotide are linked by a chemical motif selected from the group comprising natural phosphate diester, chiral phosphorothionate, chiral methyl phosphonate, chiral phosphoramidate, chiral phosphate chiral triester, chiral boranophosphate, chiral phosphoroselenoate, phosphorodithioate, phosphorothionate amidate, methylenemethylimino, 3′-amide, 3′ achiral phosphoramidate, 3′ achiral methylene phosphonates, thioformacetal, thioethyl ether, and mixtures thereof.
K. The aptamer composition according to Paragraph A-J, where said derivatives of ribonucleotides or said derivatives of deoxyribonucleotides are selected from the group comprising locked oligonucleotides, peptide oligonucleotides, glycol oligonucleotides, threose oligonucleotides, hexitol oligonucleotides, altritol oligonucleotides, butyl oligonucleotides, L-ribonucleotides, arabino oligonucleotides, 2′-fluoroarabino oligonucleotides, cyclohexene oligonucleotides, phosphorodiamidate morpholino oligonucleotides, and mixtures thereof.
L. The aptamer composition according to Paragraph A-K, further comprising at least one polymeric material, wherein said at least one polymeric material is covalently linked to said at least one oligonucleotide.
M. The aptamer composition according to Paragraph A-L, wherein said at least one polymeric material is polyethylene glycol.
N. The aptamer composition according to Paragraph A-M, wherein the nucleotides at the 5′- and 3′-ends of said at least one oligonucleotide are inverted.
O. The aptamer composition according to Paragraph A-N, wherein at least one nucleotide of said at least one oligonucleotide is fluorinated at the 2′ position of the pentose group.
P. The aptamer composition according to Paragraph A-0, wherein the pyrimidine nucleotides of said at least one oligonucleotide are fluorinated at the 2′ position of the pentose group.
Q. The aptamer composition according to Paragraph A-P, wherein said at least one oligonucleotide is covalently or non-covalently attached to one or more hair care active ingredients; wherein said one or more hair care active ingredients are selected from the group comprising: conditioning agents, brightening agents, strengthening agents, anti-fungal agents, anti-bacterial agents, anti-microbial agents, anti-dandruff agents, anti-malodor agents, perfumes, olfactory enhancement agents, anti-itch agents, cooling agents, anti-adherence agents, moisturization agents, smoothness agents, surface modification agents, antioxidants, natural extracts and essential oils, dyes, pigments, bleaches, nutrients, peptides, vitamins, enzymes, chelants, and mixtures thereof.
R. The aptamer composition according to Paragraph A-Q, wherein said hair care active ingredient is selected from the group consisting of conditioning agents.
S. The aptamer composition according to Paragraph A-R, wherein said hair care active ingredient is silicones.
T. The aptamer composition according to Paragraph A-S, wherein said at least one oligonucleotide is covalently or non-covalently attached to one or more nanomaterials.
U. A hair care composition according to Paragraph A-T, comprising at least one nucleic acid aptamer; wherein said at least one nucleic acid aptamer has a binding affinity for a hair component.
V. The hair care composition according to Paragraph A-U, wherein said hair component is selected from the group comprising: hair cuticle, hair epicuticle, hair exocuticle, hair endocuticle, hair cortex, hair keratins, hair F-layer, hair lipids, 18-methyleicosanoic acid, and mixtures thereof.
W. The hair care composition according to Paragraph A-V, wherein said hair component is hair cuticle.
X. The hair care composition according to Paragraph A-W, wherein said composition comprises at least two different nucleic acid aptamers; and wherein said at least two different nucleic acid aptamers have binding affinities for different epitopes of said hair components.
Y. A method for delivering one or more hair care active ingredients to the hair according to Paragraph A-X, comprising administering a hair care composition comprising at least one nucleic acid aptamer and one or more hair care active ingredients; wherein said at least one nucleic acid aptamer and said one or more hair care active ingredients are covalently or non-covalently attached; and wherein said at least one nucleic acid aptamer has a binding affinity for a hair component.
Z. The method according to Paragraph A-Y, wherein said hair component is hair cuticle.
AA. A method for delivering one or more hair care active ingredients to the hair according to Paragraph A-Z, comprises administering a hair care composition comprising: at least one nucleic acid aptamer and one or more nanomaterials; wherein said at least one nucleic acid aptamer and said one or more nanomaterials are covalently or non-covalently attached; and wherein said at least one nucleic acid aptamer has a binding affinity for a hair component.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While the present invention has been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An aptamer composition comprising at least one oligonucleotide consisting of: deoxyribonucleotides, ribonucleotides, derivatives of deoxyribonucleotides, derivatives of ribonucleotides, and mixtures thereof; wherein said aptamer composition has a binding affinity for a material selected from the group consisting of: undamaged hair, damaged hair, hair cuticle, hair epicuticle, hair exocuticle, hair endocuticle, hair cortex, hair keratins, hair F-layer, hair lipids, 18-methyleicosanoic acid, and mixtures thereof.

2. The aptamer composition of claim 1, wherein said aptamer composition has a binding affinity for damaged hair.

3. The aptamer composition of claim 2, wherein said aptamer composition has a higher binding affinity for damaged hair than for undamaged hair.

4. The aptamer composition of claim 1, comprising at least one oligonucleotide selected from the group consisting of oligonucleotides with at least 50% nucleotide sequence identity to sequences selected from the group consisting of SEQ ID NO 1 to SEQ ID NO 200 and SEQ ID NO 214 to SEQ ID NO 220.

5. The aptamer composition of claim 1, comprising at least one oligonucleotide selected from the group consisting of SEQ ID NO 1 to SEQ ID NO 200 and SEQ ID NO 214 to SEQ ID NO 220.

6. The aptamer composition of claim 1, comprising at least one oligonucleotide selected from the group consisting of SEQ ID NO 1, SEQ ID NO 2, SEQ ID NO 101, SEQ ID NO 102, and SEQ ID NO 214 to SEQ ID NO 220.

7. The aptamer composition of claim 1, wherein said at least one oligonucleotide comprises one or more motifs selected from the group consisting of SEQ ID NO 201 to SEQ ID NO 213.

8. The aptamer composition of claim 1, wherein said at least one oligonucleotide comprises natural or non-natural nucleobases.

9. The aptamer composition of claim 8, wherein said non-natural nucleobases are selected from the group comprising hypoxanthine, xanthine, 7-methylguanine, 5,6-dihydrouracil, 5-5-methylcytosine, 5-hydroxymethylcytosine, thiouracil, 1-methylhypoxanthine, 6-methylisoquinoline-1-thione-2-yl, 3-methoxy-2-naphthyl, 5-propynyluracil-1-yl, 5-methylcytosin-1-yl, 2-aminoadenin-9-yl, 7-deaza-7-iodoadenin-9-yl, 7-deaza-7-propynyl-2-aminoadenin-9-yl, phenoxazinyl, phenoxazinyl-G-clam, and mixtures thereof.

10. The aptamer composition of claim 1, wherein the nucleosides of said at least one oligonucleotide are linked by a chemical motif selected from the group comprising natural phosphate diester, chiral phosphorothionate, chiral methyl phosphonate, chiral phosphoramidate, chiral phosphate chiral triester, chiral boranophosphate, chiral phosphoroselenoate, phosphorodithioate, phosphorothionate amidate, methylenemethylimino, 3′-amide, 3′ achiral phosphoramidate, 3′ achiral methylene phosphonates, thioformacetal, thioethyl ether, and mixtures thereof.

11. The aptamer composition of claim 1, where said derivatives of ribonucleotides or said derivatives of deoxyribonucleotides are selected from the group comprising locked oligonucleotides, peptide oligonucleotides, glycol oligonucleotides, threose oligonucleotides, hexitol oligonucleotides, altritol oligonucleotides, butyl oligonucleotides, L-ribonucleotides, arabino oligonucleotides, 2′-fluoroarabino oligonucleotides, cyclohexene oligonucleotides, phosphorodiamidate morpholino oligonucleotides, and mixtures thereof.

12. The aptamer composition of claim 1, further comprising at least one polymeric material, wherein said at least one polymeric material is covalently linked to said at least one oligonucleotide.

13. The aptamer composition of claim 11, wherein said at least one polymeric material is polyethylene glycol.

14. The aptamer composition of claim 1, wherein the nucleotides at the 5′- and 3′-ends of said at least one oligonucleotide are inverted.

15. The aptamer composition of claim 1, wherein at least one nucleotide of said at least one oligonucleotide is fluorinated at the 2′ position of the pentose group.

16. The aptamer composition of claim 1, wherein the pyrimidine nucleotides of said at least one oligonucleotide are fluorinated at the 2′ position of the pentose group.

17. The aptamer composition of claim 1, wherein said at least one oligonucleotide is covalently or non-covalently attached to one or more hair care active ingredients; wherein said one or more hair care active ingredients are selected from the group comprising: conditioning agents, brightening agents, strengthening agents, anti-fungal agents, anti-bacterial agents, anti-microbial agents, anti-dandruff agents, anti-malodor agents, perfumes, olfactory enhancement agents, anti-itch agents, cooling agents, anti-adherence agents, moisturization agents, smoothness agents, surface modification agents, antioxidants, natural extracts and essential oils, dyes, pigments, bleaches, nutrients, peptides, vitamins, enzymes, chelants, and mixtures thereof.

18. The aptamer composition of claim 17, wherein said hair care active ingredient is selected from the group consisting of conditioning agents.

19. The aptamer composition of claim 18, wherein said hair care active ingredient is silicones.

20. The aptamer composition of claim 1, wherein said at least one oligonucleotide is covalently or non-covalently attached to one or more nanomaterials.

21. A hair care composition comprising at least one nucleic acid aptamer; wherein said at least one nucleic acid aptamer has a binding affinity for a hair component.

22. The hair care composition of claim 21, wherein said hair component is selected from the group comprising: hair cuticle, hair epicuticle, hair exocuticle, hair endocuticle, hair cortex, hair keratins, hair F-layer, hair lipids, 18-methyleicosanoic acid, and mixtures thereof.

23. The hair care composition of claim 21, wherein said hair component is hair cuticle.

24. The hair care composition of claim 21, wherein said composition comprises at least two different nucleic acid aptamers; and wherein said at least two different nucleic acid aptamers have binding affinities for different epitopes of said hair components.

25. A method for delivering one or more hair care active ingredients to the hair comprising administering a hair care composition comprising at least one nucleic acid aptamer and one or more hair care active ingredients; wherein said at least one nucleic acid aptamer and said one or more hair care active ingredients are covalently or non-covalently attached; and wherein said at least one nucleic acid aptamer has a binding affinity for a hair component.

26. The method of claim 25, wherein said hair component is hair cuticle.

27. A method for delivering one or more hair care active ingredients to the hair comprises administering a hair care composition comprising: at least one nucleic acid aptamer and one or more nanomaterials; wherein said at least one nucleic acid aptamer and said one or more nanomaterials are covalently or non-covalently attached; and wherein said at least one nucleic acid aptamer has a binding affinity for a hair component.


SEQUENCE LISTING (220 sequences)

SEQ ID NO: 1 - DNA, 79 bp, Artificial Sequence
  Synthetic Aptamer Sequence
aactacatggtatgtggtgaactgaatatggattacaagtttcagatcgagcactcccattcagacgtacaatgtaccc

SEQ ID NO: 2 - DNA, 79 bp, Artificial Sequence
  Synthetic Aptamer Sequence
aactacatggtatgtggtgaactaggggaaccttagtaaaccacgacccaggatgtgctatcggacgtacaatgtaccc

SEQ ID NO: 3 - DNA, 79 bp, Artificial Sequence
  Synthetic Aptamer Sequence
aactacatggtatgtggtgaactcaacttttaagcaagctgtctaccacggaggcagtatcacgacgtacaatgtaccc

SEQ ID NO: 4 - DNA, 79 bp, Artificial Sequence
  Synthetic Aptamer Sequence
aactacatggtatgtggtgaactaccgaaatccaaaaagcagaaccaccgacctacaatggcggacgtacaatgtaccc

SEQ ID NO: 5 - DNA, 79 bp, Artificial Sequence
  Synthetic Aptamer Sequence
aactacatggtatgtggtgaactgccccgacgaaccaaggagatcgcagttactactaccgtagacgtacaatgtaccc

SEQ ID NO: 6 - DNA, 79 bp, Artificial Sequence
  Synthetic Aptamer Sequence
aactacatggtatgtggtgaactgccgaaagaggccatgtaaaccacgtataagtagcccatagacgtacaatgtaccc

SEQ ID NO: 7 - DNA, 79 bp, Artificial Sequence
  Synthetic Aptamer Sequence
aactacatggtatgtggtgaactcagcacgagaagttcgcgccacagacagtgcctaagccaggacgtacaatgtaccc

SEQ ID NO: 8 - DNA, 79 bp, Artificial Sequence
  Synthetic Aptamer Sequence
aactacatggtatgtggtgaactgagaatgaaacagcagttttgcgacacggccaacgtattagacgtacaatgtaccc

SEQ ID NO: 9 - DNA, 79 bp, Artificial Sequence
  Synthetic Aptamer Sequence
aactacatggtatgtggtgaactcagacaacctgctcagttagaccgatttgacgagcaacacgacgtacaatgtaccc

SEQ ID NO: 10 - DNA, 79 bp, Artificial Sequence
  Synthetic Aptamer Sequence
aactacatggtatgtggtgaactttcgcggatattgctgatatattgcccacagacgtatggagacgtacaatgtaccc

SEQ ID NO: 11 - DNA, 79 bp, Artificial Sequence
  Synthetic Aptamer Sequence
aactacatggtatgtggtgaactagttccacaagatgcagaagcatacaccgcgtctagaagcgacgtacaatgtaccc

SEQ ID NO: 12 - DNA, 79 bp, Artificial Sequence
  Synthetic Aptamer Sequence
aactacatggtatgtggtgaacttcaaagttatagcactatcagacagcagagaccatgacaagacgtacaatgtaccc

SEQ ID NO: 13 - DNA, 79 bp, Artificial Sequence
  Synthetic Aptamer Sequence
aactacatggtatgtggtgaactaagcggcccgcaaaacgtttgcgaagcggttcatctaccagacgtacaatgtaccc

SEQ ID NO: 14 - DNA, 79 bp, Artificial Sequence
  Synthetic Aptamer Sequence
aactacatggtatgtggtgaacttccaggtcgcgtaggtctaacgttcctgaacagtttcatcgacgtacaatgtaccc

SEQ ID NO: 15 - DNA, 79 bp, Artificial Sequence
  Synthetic Aptamer Sequence
aactacatggtatgtggtgaactaagacaaatgtcatgcaccatatacagggccagccagctagacgtacaatgtaccc

SEQ ID NO: 16 - DNA, 79 bp, Artificial Sequence
  Synthetic Aptamer Sequence
aactacatggtatgtggtgaactaccagagaacatacccaggcaatttatatcgctctaatgagacgtacaatgtaccc

SEQ ID NO: 17 - DNA, 79 bp, Artificial Sequence
  Synthetic Aptamer Sequence
aactacatggtatgtggtgaactgagcgatgacgaaaagtgtaatgccaagaccacgcggttagacgtacaatgtaccc

SEQ ID NO: 18 - DNA, 79 bp, Artificial Sequence
  Synthetic Aptamer Sequence
aactacatggtatgtggtgaacttacgaaggcagctgcataagatacagagagatccaccactgacgtacaatgtaccc

SEQ ID NO: 19 - DNA, 79 bp, Artificial Sequence
  Synthetic Aptamer Sequence
aactacatggtatgtggtgaactttaatgattaacgattaacttcaatgtttaccacgccgaggacgtacaatgtaccc

SEQ ID NO: 20 - DNA, 79 bp, Artificial Sequence
  Synthetic Aptamer Sequence
aactacatggtatgtggtgaactgacctatatccctgcgatctgcagaggaatagtgaagttcgacgtacaatgtaccc

SEQ ID NO: 21 - DNA, 79 bp, Artificial Sequence
  Synthetic Aptamer Sequence
aactacatggtatgtggtgaactacaaagaccgcatcgatctatgccatggactaattcagcagacgtacaatgtaccc

SEQ ID NO: 22 - DNA, 79 bp, Artificial Sequence
  Synthetic Aptamer Sequence
aactacatggtatgtggtgaactgatagcgggctccagcaattaccaaaacttaccagcgtcagacgtacaatgtaccc

SEQ ID NO: 23 - DNA, 79 bp, Artificial Sequence
  Synthetic Aptamer Sequence
aactacatggtatgtggtgaactctatcacccacgttactacgtcactacgagcaactcatgagacgtacaatgtaccc

SEQ ID NO: 24 - DNA, 79 bp, Artificial Sequence
  Synthetic Aptamer Sequence
aactacatggtatgtggtgaactgcagccgatacgcttagctggttcatattcaccccccaaagacgtacaatgtaccc

SEQ ID NO: 25 - DNA, 79 bp, Artificial Sequence
  Synthetic Aptamer Sequence
aactacatggtatgtggtgaactctgatttcagaatctcggaacccgcccgtcatccattatggacgtacaatgtaccc

SEQ ID NO: 26 - DNA, 79 bp, Artificial Sequence
  Synthetic Aptamer Sequence
aactacatggtatgtggtgaacttccacacactgagaaggcacaagcaacgccgtatagtcatgacgtacaatgtaccc

SEQ ID NO: 27 - DNA, 79 bp, Artificial Sequence
  Synthetic Aptamer Sequence
aactacatggtatgtggtgaactgctttgaactataaagcaaatcagcacgcgttgccacgaagacgtacaatgtaccc

SEQ ID NO: 28 - DNA, 79 bp, Artificial Sequence
  Synthetic Aptamer Sequence
aactacatggtatgtggtgaactcgtgaggcgtaacttaacatggagcctctactgatccacagacgtacaatgtaccc

SEQ ID NO: 29 - DNA, 79 bp, Artificial Sequence
  Synthetic Aptamer Sequence
aactacatggtatgtggtgaactagcatatgatttgcagcatcatatataaaactgttccccagacgtacaatgtaccc

SEQ ID NO: 30 - DNA, 79 bp, Artificial Sequence
  Synthetic Aptamer Sequence
aactacatggtatgtggtgaactggagcactttagggtgatagtgacagaccaccgtaccacagacgtacaatgtaccc

SEQ ID NO: 31 - DNA, 79 bp, Artificial Sequence
  Synthetic Aptamer Sequence
aactacatggtatgtggtgaacttgacctaatcatccaaatggagttttacagaaactgcgaggacgtacaatgtaccc

SEQ ID NO: 32 - DNA, 79 bp, Artificial Sequence
  Synthetic Aptamer Sequence
aactacatggtatgtggtgaactggagcgtgacaaacactggtccgacgcagcacactcacctgacgtacaatgtaccc

SEQ ID NO: 33 - DNA, 79 bp, Artificial Sequence
  Synthetic Aptamer Sequence
aactacatggtatgtggtgaactcgaggcgtcattagcccacagcatggcacatactaagagagacgtacaatgtaccc

SEQ ID NO: 34 - DNA, 79 bp, Artificial Sequence
  Synthetic Aptamer Sequence
aactacatggtatgtggtgaactcaaccagaaacctagaggtaaataggaattgagggagacagacgtacaatgtaccc

SEQ ID NO: 35 - DNA, 79 bp, Artificial Sequence
  Synthetic Aptamer Sequence
aactacatggtatgtggtgaactcgcgcattcttgaacagataatactcggcgcaagataccggacgtacaatgtaccc

SEQ ID NO: 36 - DNA, 79 bp, Artificial Sequence
  Synthetic Aptamer Sequence
aactacatggtatgtggtgaactactttgacggtgccaagagaactagcttaagtccgtgttcgacgtacaatgtaccc

SEQ ID NO: 37 - DNA, 79 bp, Artificial Sequence
  Synthetic Aptamer Sequence
aactacatggtatgtggtgaactaataacaaggtgccaaaaacctctcagaaacaagaaccccgacgtacaatgtaccc

SEQ ID NO: 38 - DNA, 79 bp, Artificial Sequence
  Synthetic Aptamer Sequence
aactacatggtatgtggtgaacttcaacgcgggagtcgacaaccaactaccaaactgcggagagacgtacaatgtaccc

SEQ ID NO: 39 - DNA, 79 bp, Artificial Sequence
  Synthetic Aptamer Sequence
aactacatggtatgtggtgaacttaatgagcgcacatatacaagtaagtagcagcgagaatcagacgtacaatgtaccc

SEQ ID NO: 40 - DNA, 79 bp, Artificial Sequence
  Synthetic Aptamer Sequence
aactacatggtatgtggtgaactcacagacattagaatgtgactcgccgcaaaccgatagacagacgtacaatgtaccc

SEQ ID NO: 41 - DNA, 79 bp, Artificial Sequence
  Synthetic Aptamer Sequence
aactacatggtatgtggtgaactggacaacgtttaaatgtgccgaaaccgcatagacgtattggacgtacaatgtaccc

SEQ ID NO: 42 - DNA, 79 bp, Artificial Sequence
  Synthetic Aptamer Sequence
aactacatggtatgtggtgaactcggacaaagagctcaatcctggacagcacgtaggtatgtagacgtacaatgtaccc

SEQ ID NO: 43 - DNA, 79 bp, Artificial Sequence
  Synthetic Aptamer Sequence
aactacatggtatgtggtgaactaggtatcgccagactatatagtaagtcgaacagaaccaccgacgtacaatgtaccc

SEQ ID NO: 44 - DNA, 79 bp, Artificial Sequence
  Synthetic Aptamer Sequence
aactacatggtatgtggtgaactcctcgactgtcatcgcatccaagcgtgcaccagaagctcagacgtacaatgtaccc

SEQ ID NO: 45 - DNA, 79 bp, Artificial Sequence
  Synthetic Aptamer Sequence
aactacatggtatgtggtgaactactgtttatgtgccgatgtataagcaagtattcgatcaccgacgtacaatgtaccc

SEQ ID NO: 46 - DNA, 79 bp, Artificial Sequence
  Synthetic Aptamer Sequence
aactacatggtatgtggtgaactgtgttgaactgatcatggccctgatcgctcaacggctcaagacgtacaatgtaccc

SEQ ID NO: 47 - DNA, 79 bp, Artificial Sequence
  Synthetic Aptamer Sequence
aactacatggtatgtggtgaactaaggcgctatcgggaacgcagccctttctaccaaacccaagacgtacaatgtaccc

SEQ ID NO: 48 - DNA, 79 bp, Artificial Sequence
  Synthetic Aptamer Sequence
aactacatggtatgtggtgaactcgagcagaaggtccaccggcaacgcaattaccaagaatccgacgtacaatgtaccc

SEQ ID NO: 49 - DNA, 79 bp, Artificial Sequence
  Synthetic Aptamer Sequence
aactacatggtatgtggtgaactttagtttagatcaacacaccctgattgcaactgctgcatagacgtacaatgtaccc

SEQ ID NO: 50 - DNA, 79 bp, Artificial Sequence
  Synthetic Aptamer Sequence
aactacatggtatgtggtgaactaaagaggcagacgcgtaatcatagcagccaaaatagacacgacgtacaatgtaccc

SEQ ID NO: 51 - DNA, 79 bp, Artificial Sequence
  Synthetic Aptamer Sequence
aactacatggtatgtggtgaactgtaagtcccacaaatgcattcaggctagctcatgtagcacgacgtacaatgtaccc

SEQ ID NO: 52 - DNA, 79 bp, Artificial Sequence
  Synthetic Aptamer Sequence
aactacatggtatgtggtgaactttcattgcctgcgtaaaccacacggtccgttataaacttagacgtacaatgtaccc

SEQ ID NO: 53 - DNA, 79 bp, Artificial Sequence
  Synthetic Aptamer Sequence
aactacatggtatgtggtgaacttgaccaaaccagcctatgagtgataagcttctgtgcagtagacgtacaatgtaccc

SEQ ID NO: 54 - DNA, 79 bp, Artificial Sequence
  Synthetic Aptamer Sequence
aactacatggtatgtggtgaactggctgagacgaaccactagggtgatcaccaaacccgctcagacgtacaatgtaccc

SEQ ID NO: 55 - DNA, 79 bp, Artificial Sequence
  Synthetic Aptamer Sequence
aactacatggtatgtggtgaacttgacaaataaggatagaatcaacatcacaagcaggcagttgacgtacaatgtaccc

SEQ ID NO: 56 - DNA, 79 bp, Artificial Sequence
  Synthetic Aptamer Sequence
aactacatggtatgtggtgaacttaaatttgccacaatatcttggccccatagaagggccgtcgacgtacaatgtaccc

SEQ ID NO: 57 - DNA, 79 bp, Artificial Sequence
  Synthetic Aptamer Sequence
aactacatggtatgtggtgaactaataacacataacacacgcgaaccaatctcccggcccaaagacgtacaatgtaccc

SEQ ID NO: 58 - DNA, 79 bp, Artificial Sequence
  Synthetic Aptamer Sequence
aactacatggtatgtggtgaacttgatgccaatgacaacgccacacgttcgacacacatacacgacgtacaatgtaccc

SEQ ID NO: 59 - DNA, 79 bp, Artificial Sequence
  Synthetic Aptamer Sequence
aactacatggtatgtggtgaactaaaacgggtttagatcatcacgaggactcatgcgggatttgacgtacaatgtaccc

SEQ ID NO: 60 - DNA, 79 bp, Artificial Sequence
  Synthetic Aptamer Sequence
aactacatggtatgtggtgaactgaaatcgccacagagtctttgcggaagagcgtgaaaagcagacgtacaatgtaccc

SEQ ID NO: 61 - DNA, 79 bp, Artificial Sequence
  Synthetic Aptamer Sequence
aactacatggtatgtggtgaactccccgatctccatcgatcttcaagataggaaaggacaccagacgtacaatgtaccc

SEQ ID NO: 62 - DNA, 79 bp, Artificial Sequence
  Synthetic Aptamer Sequence
aactacatggtatgtggtgaactgtctcgaggttcataagctatggaaacaagcaccgcatatgacgtacaatgtaccc

SEQ ID NO: 63 - DNA, 79 bp, Artificial Sequence
  Synthetic Aptamer Sequence
aactacatggtatgtggtgaactaccgtcaaatggtgactttcgagtttgccacacctaagaggacgtacaatgtaccc

SEQ ID NO: 64 - DNA, 79 bp, Artificial Sequence
  Synthetic Aptamer Sequence
aactacatggtatgtggtgaactgtcccgcaatccaaaatcgcgcacaagagcccacagccaggacgtacaatgtaccc

SEQ ID NO: 65 - DNA, 79 bp, Artificial Sequence
  Synthetic Aptamer Sequence
aactacatggtatgtggtgaactggccccgtctaggacgaccaacacctgccgtcgactgtgagacgtacaatgtaccc

SEQ ID NO: 66 - DNA, 79 bp, Artificial Sequence
  Synthetic Aptamer Sequence
aactacatggtatgtggtgaactcgtctgagccaccttaaccagatttgataacccacagcgagacgtacaatgtaccc

SEQ ID NO: 67 - DNA, 79 bp, Artificial Sequence
  Synthetic Aptamer Sequence
aactacatggtatgtggtgaactatgtgaattcaaggaattgcagccacatagcgccgaatacgacgtacaatgtaccc

SEQ ID NO: 68 - DNA, 79 bp, Artificial Sequence
  Synthetic Aptamer Sequence
aactacatggtatgtggtgaactggaggacgtcgtaagatgttacaaaggcactcccgaactagacgtacaatgtaccc

SEQ ID NO: 69 - DNA, 79 bp, Artificial Sequence
  Synthetic Aptamer Sequence
aactacatggtatgtggtgaactgccattgacagagaggagaaatcttttgagcagtgagcacgacgtacaatgtaccc

SEQ ID NO: 70 - DNA, 79 bp, Artificial Sequence
  Synthetic Aptamer Sequence
aactacatggtatgtggtgaactaactttgcggcacccacaagagttcgtaaaagcagacaccgacgtacaatgtaccc

SEQ ID NO: 71 - DNA, 79 bp, Artificial Sequence
  Synthetic Aptamer Sequence
aactacatggtatgtggtgaacttgtggcggcgaacacaccatgagcacctcacatgaccgtggacgtacaatgtaccc

SEQ ID NO: 72 - DNA, 79 bp, Artificial Sequence
  Synthetic Aptamer Sequence
aactacatggtatgtggtgaactcaacgaacagtagctatgataacagccttcgacgtgtccagacgtacaatgtaccc

SEQ ID NO: 73 - DNA, 79 bp, Artificial Sequence
  Synthetic Aptamer Sequence
aactacatggtatgtggtgaactgcccttacggcacatacagtgactcatggcggcagctaacgacgtacaatgtaccc

SEQ ID NO: 74 - DNA, 79 bp, Artificial Sequence
  Synthetic Aptamer Sequence
aactacatggtatgtggtgaactgtagcattgccgagagctcacctgttttacacgcgagttagacgtacaatgtaccc

SEQ ID NO: 75 - DNA, 79 bp, Artificial Sequence
  Synthetic Aptamer Sequence
aactacatggtatgtggtgaactgatccgtaggtcacacctttatgccatccgggaccaattcgacgtacaatgtaccc

SEQ ID NO: 76 - DNA, 79 bp, Artificial Sequence
  Synthetic Aptamer Sequence
aactacatggtatgtggtgaactcgatctgtacgagactcgatcctacgcacagcaccccagtgacgtacaatgtaccc

SEQ ID NO: 77 - DNA, 79 bp, Artificial Sequence
  Synthetic Aptamer Sequence
aactacatggtatgtggtgaacttcctacaaagctatttgcaggtcggacgtggataccaattgacgtacaatgtaccc

SEQ ID NO: 78 - DNA, 79 bp, Artificial Sequence
  Synthetic Aptamer Sequence
aactacatggtatgtggtgaactaggcaaaaacaaccttaaccttgagcccacaagccagatagacgtacaatgtaccc

SEQ ID NO: 79 - DNA, 79 bp, Artificial Sequence
  Synthetic Aptamer Sequence
aactacatggtatgtggtgaacttccgaaatgaaaaaagttacccgacacggccaaggctagtgacgtacaatgtaccc

SEQ ID NO: 80 - DNA, 79 bp, Artificial Sequence
  Synthetic Aptamer Sequence
aactacatggtatgtggtgaactagaacggagaagtccggtccgagtatctttaaataccagcgacgtacaatgtaccc

SEQ ID NO: 81 - DNA, 79 bp, Artificial Sequence
  Synthetic Aptamer Sequence
aactacatggtatgtggtgaactctgaatgcgagatgtacaacacggatcgacgtagcttattgacgtacaatgtaccc

SEQ ID NO: 82 - DNA, 79 bp, Artificial Sequence
  Synthetic Aptamer Sequence
aactacatggtatgtggtgaactgcagtacaaaatgcggtttctttcacaacgattagtagtcgacgtacaatgtaccc

SEQ ID NO: 83 - DNA, 79 bp, Artificial Sequence
  Synthetic Aptamer Sequence
aactacatggtatgtggtgaactaggaactacaacgttggtcctgaaatcacaaccatctaaagacgtacaatgtaccc

SEQ ID NO: 84 - DNA, 79 bp, Artificial Sequence
  Synthetic Aptamer Sequence
aactacatggtatgtggtgaacttccaaaccaaattaggatgatccagctcgccacagccaaggacgtacaatgtaccc

SEQ ID NO: 85 - DNA, 79 bp, Artificial Sequence
  Synthetic Aptamer Sequence
aactacatggtatgtggtgaactcggaagaaggaggccacatcctggagcaacaagacgagaagacgtacaatgtaccc

SEQ ID NO: 86 - DNA, 79 bp, Artificial Sequence
  Synthetic Aptamer Sequence
aactacatggtatgtggtgaactatgctacacggagaccgaagctcttacgagatagttctcagacgtacaatgtaccc

SEQ ID NO: 87 - DNA, 79 bp, Artificial Sequence
  Synthetic Aptamer Sequence
aactacatggtatgtggtgaactagaggcggcttaaccctacagctaccccgacatcaagtccgacgtacaatgtaccc

SEQ ID NO: 88 - DNA, 79 bp, Artificial Sequence
  Synthetic Aptamer Sequence
aactacatggtatgtggtgaacttggatagtgtggctgaaataccaattaaccaaaccaatgcgacgtacaatgtaccc

SEQ ID NO: 89 - DNA, 79 bp, Artificial Sequence
  Synthetic Aptamer Sequence
aactacatggtatgtggtgaactaacaaaaccgaatctgtggagcgccacaacccaaatactagacgtacaatgtaccc

SEQ ID NO: 90 - DNA, 79 bp, Artificial Sequence
  Synthetic Aptamer Sequence
aactacatggtatgtggtgaactgggaaacagaagaccacattactcaatgcgaatatcgactgacgtacaatgtaccc

SEQ ID NO: 91 - DNA, 79 bp, Artificial Sequence
  Synthetic Aptamer Sequence
aactacatggtatgtggtgaactatggaaaaaaggatggtcccacctcccaaaaccattgtcagacgtacaatgtaccc

SEQ ID NO: 92 - DNA, 79 bp, Artificial Sequence
  Synthetic Aptamer Sequence
aactacatggtatgtggtgaactatagaaactgaccaccagtcacaccctgagaagaagcagagacgtacaatgtaccc

SEQ ID NO: 93 - DNA, 79 bp, Artificial Sequence
  Synthetic Aptamer Sequence
aactacatggtatgtggtgaactaactgacatggtctctgagacggccatagagtgttcaaaagacgtacaatgtaccc

SEQ ID NO: 94 - DNA, 79 bp, Artificial Sequence
  Synthetic Aptamer Sequence
aactacatggtatgtggtgaactctaataaacggcgggctgaattagagacgacacaaccgcagacgtacaatgtaccc

SEQ ID NO: 95 - DNA, 79 bp, Artificial Sequence
  Synthetic Aptamer Sequence
aactacatggtatgtggtgaacttaagcggcccttaggagcgttggtaccacattcatggagagacgtacaatgtaccc

SEQ ID NO: 96 - DNA, 79 bp, Artificial Sequence
  Synthetic Aptamer Sequence
aactacatggtatgtggtgaactgcagagaggcggttagcccagaaatcaaccacgtgccatagacgtacaatgtaccc

SEQ ID NO: 97 - DNA, 79 bp, Artificial Sequence
  Synthetic Aptamer Sequence
aactacatggtatgtggtgaactgtgatgacgaaggtcataggtcagccaacatgcctgtgaggacgtacaatgtaccc

SEQ ID NO: 98 - DNA, 79 bp, Artificial Sequence
  Synthetic Aptamer Sequence
aactacatggtatgtggtgaactttccgtatatcggaccggtaagtctacctaacatacgtgagacgtacaatgtaccc

SEQ ID NO: 99 - DNA, 79 bp, Artificial Sequence
  Synthetic Aptamer Sequence
aactacatggtatgtggtgaactagtcggcaaaggaggatccacaacataacgagagtaactggacgtacaatgtaccc

SEQ ID NO: 100 - DNA, 79 bp, Artificial Sequence
  Synthetic Aptamer Sequence
aactacatggtatgtggtgaacttaaagttaccctgagcaatgcagcgacgaaataacgttgagacgtacaatgtaccc

SEQ ID NO: 101 - DNA, 79 bp, Artificial Sequence
  Synthetic Aptamer Sequence
aactacatggtatgtggtgaacttagtgggatttattcactatgtacaccgatgacaacagtagacgtacaatgtaccc

SEQ ID NO: 102 - DNA, 79 bp, Artificial Sequence
  Synthetic Aptamer Sequence
aactacatggtatgtggtgaactgcagaacatgaccaaaagaggaaaggtatagctgctatcagacgtacaatgtaccc

SEQ ID NO: 103 - DNA, 79 bp, Artificial Sequence
  Synthetic Aptamer Sequence
aactacatggtatgtggtgaacttagtcacgatatcgtggcccagaacctcaatcatgcaaaagacgtacaatgtaccc

SEQ ID NO: 104 - DNA, 79 bp, Artificial Sequence
  Synthetic Aptamer Sequence
aactacatggtatgtggtgaacttcagcggtgaacacatccaatcaagaaggccactatacgagacgtacaatgtaccc

SEQ ID NO: 105 - DNA, 79 bp, Artificial Sequence
  Synthetic Aptamer Sequence
aactacatggtatgtggtgaactctgaatgatgaactgtatccgaacaccaaaccaaatccctgacgtacaatgtaccc

SEQ ID NO: 106 - DNA, 79 bp, Artificial Sequence
  Synthetic Aptamer Sequence
aactacatggtatgtggtgaactgctcgatagacaggcctaaaacccccggacgaacctttcagacgtacaatgtaccc

SEQ ID NO: 107 - DNA, 79 bp, Artificial Sequence
  Synthetic Aptamer Sequence
aactacatggtatgtggtgaactaaatatttagaattctggttcacgacaacatgaacaggtggacgtacaatgtaccc

SEQ ID NO: 108 - DNA, 79 bp, Artificial Sequence
  Synthetic Aptamer Sequence
aactacatggtatgtggtgaactaaggagaacgaagtgcacttgcaacttcactatcagcacagacgtacaatgtaccc

SEQ ID NO: 109 - DNA, 79 bp, Artificial Sequence
  Synthetic Aptamer Sequence
aactacatggtatgtggtgaactgaatccgaacacaagaacatgacggaaggcttataccgatgacgtacaatgtaccc

SEQ ID NO: 110 - DNA, 79 bp, Artificial Sequence
  Synthetic Aptamer Sequence
aactacatggtatgtggtgaactgccagggaccttcaaccgatgaggtgacagactgacaattgacgtacaatgtaccc

SEQ ID NO: 111 - DNA, 79 bp, Artificial Sequence
  Synthetic Aptamer Sequence
aactacatggtatgtggtgaactatcgatggatctccaatcgacagtcactctgaaccctttagacgtacaatgtaccc

SEQ ID NO: 112 - DNA, 79 bp, Artificial Sequence
  Synthetic Aptamer Sequence
aactacatggtatgtggtgaactacgaagggaactgctcaccaacaacacgcccgtaggactcgacgtacaatgtaccc

SEQ ID NO: 113 - DNA, 79 bp, Artificial Sequence
  Synthetic Aptamer Sequence
aactacatggtatgtggtgaactagatgaagacaccgacttaagccgacgtaatcttctagaagacgtacaatgtaccc

SEQ ID NO: 114 - DNA, 79 bp, Artificial Sequence
  Synthetic Aptamer Sequence
aactacatggtatgtggtgaactacattatgataagccgagtccacgtgcttcatacaatctagacgtacaatgtaccc

SEQ ID NO: 115 - DNA, 79 bp, Artificial Sequence
  Synthetic Aptamer Sequence
aactacatggtatgtggtgaacttgtaaaagttgaggacataccaacgctaaagaacgagctagacgtacaatgtaccc

SEQ ID NO: 116 - DNA, 79 bp, Artificial Sequence
  Synthetic Aptamer Sequence
aactacatggtatgtggtgaactatcccctcgactccagcgtttcagaatcgcttaccagtaggacgtacaatgtaccc

SEQ ID NO: 117 - DNA, 79 bp, Artificial Sequence
  Synthetic Aptamer Sequence
aactacatggtatgtggtgaactatcggagcgacgacgcgctaataagcccactatggatgtagacgtacaatgtaccc

SEQ ID NO: 118 - DNA, 79 bp, Artificial Sequence
  Synthetic Aptamer Sequence
aactacatggtatgtggtgaactggataactggatcaccgactttgaaacgctccatgtggatgacgtacaatgtaccc

SEQ ID NO: 119 - DNA, 79 bp, Artificial Sequence
  Synthetic Aptamer Sequence
aactacatggtatgtggtgaactaccaaaaagcagagcctggcacagcgctacaaggcagatagacgtacaatgtaccc

SEQ ID NO: 120 - DNA, 79 bp, Artificial Sequence
  Synthetic Aptamer Sequence
aactacatggtatgtggtgaacttgcactatgacaacctctagactgctgcatttgaaaccacgacgtacaatgtaccc

SEQ ID NO: 121 - DNA, 79 bp, Artificial Sequence
  Synthetic Aptamer Sequence
aactacatggtatgtggtgaactagttagaccactcacagtccattaaggcagctaggagccagacgtacaatgtaccc

SEQ ID NO: 122 - DNA, 79 bp, Artificial Sequence
  Synthetic Aptamer Sequence
aactacatggtatgtggtgaacttgagcagagacgttcagcgaaggtctccgccttcgaatccgacgtacaatgtaccc

SEQ ID NO: 123 - DNA, 79 bp, Artificial Sequence
  Synthetic Aptamer Sequence
aactacatggtatgtggtgaactcctctgagcataagtcgaggaaaaaccgccgaccaatatagacgtacaatgtaccc

SEQ ID NO: 124 - DNA, 79 bp, Artificial Sequence
  Synthetic Aptamer Sequence
aactacatggtatgtggtgaacttccaaatggacacacccgcatagaccgagttgtacctgaagacgtacaatgtaccc

SEQ ID NO: 125 - DNA, 79 bp, Artificial Sequence
  Synthetic Aptamer Sequence
aactacatggtatgtggtgaactatgagagaacacgggcatacttgcatcccatatacgtttagacgtacaatgtaccc

SEQ ID NO: 126 - DNA, 79 bp, Artificial Sequence
  Synthetic Aptamer Sequence
aactacatggtatgtggtgaacttatcgccgtaagattctgacaaacccacggaatcacccaagacgtacaatgtaccc

SEQ ID NO: 127 - DNA, 79 bp, Artificial Sequence
  Synthetic Aptamer Sequence
aactacatggtatgtggtgaactcgagaagcagcgccatacctactgacggacacatacgaaggacgtacaatgtaccc

SEQ ID NO: 128 - DNA, 79 bp, Artificial Sequence
  Synthetic Aptamer Sequence
aactacatggtatgtggtgaacttcagacttaaggatacgctgagccaacaccacatcatcgagacgtacaatgtaccc

SEQ ID NO: 129 - DNA, 79 bp, Artificial Sequence
  Synthetic Aptamer Sequence
aactacatggtatgtggtgaactcggatttcgcagaggaattgagctgcagatcccgagcagagacgtacaatgtaccc

SEQ ID NO: 130 - DNA, 79 bp, Artificial Sequence
  Synthetic Aptamer Sequence
aactacatggtatgtggtgaactcaatgagctcgaaacgcggaaatccatgccatggaagacggacgtacaatgtaccc

SEQ ID NO: 131 - DNA, 79 bp, Artificial Sequence
  Synthetic Aptamer Sequence
aactacatggtatgtggtgaactaattccacggaaagagttaagcagcccgcgttacatgagtgacgtacaatgtaccc

SEQ ID NO: 132 - DNA, 79 bp, Artificial Sequence
  Synthetic Aptamer Sequence
aactacatggtatgtggtgaactattacgacagaactgttgcccagtctccagcgcgctcacggacgtacaatgtaccc

SEQ ID NO: 133 - DNA, 79 bp, Artificial Sequence
  Synthetic Aptamer Sequence
aactacatggtatgtggtgaactgaggcgaaaaagcgcacaattaagaccacaagtcagtgcagacgtacaatgtaccc

SEQ ID NO: 134 - DNA, 79 bp, Artificial Sequence
  Synthetic Aptamer Sequence
aactacatggtatgtggtgaactaaaatagtttggtcttatcccaaatacgcaaagtgtcttggacgtacaatgtaccc

SEQ ID NO: 135 - DNA, 79 bp, Artificial Sequence
  Synthetic Aptamer Sequence
aactacatggtatgtggtgaactttaagtagtcacgttagagtccacggcacccgcatacaatgacgtacaatgtaccc

SEQ ID NO: 136 - DNA, 79 bp, Artificial Sequence
  Synthetic Aptamer Sequence
aactacatggtatgtggtgaacttgctaggttaggaagaaagacatttttagtcaccacacaggacgtacaatgtaccc

SEQ ID NO: 137 - DNA, 79 bp, Artificial Sequence
  Synthetic Aptamer Sequence
aactacatggtatgtggtgaactcagctagctccgccagaacagtaaccaccacatcagcagagacgtacaatgtaccc

SEQ ID NO: 138 - DNA, 79 bp, Artificial Sequence
  Synthetic Aptamer Sequence
aactacatggtatgtggtgaacttgacacaaatcatgggatggaatcataaaggttgttcacagacgtacaatgtaccc

SEQ ID NO: 139 - DNA, 79 bp, Artificial Sequence
  Synthetic Aptamer Sequence
aactacatggtatgtggtgaactcggccttacggaggaagggaagtacatccactaccgagttgacgtacaatgtaccc

SEQ ID NO: 140 - DNA, 79 bp, Artificial Sequence
  Synthetic Aptamer Sequence
aactacatggtatgtggtgaactcgaatttgacctgcattggattctggtccttttgccacaagacgtacaatgtaccc

SEQ ID NO: 141 - DNA, 79 bp, Artificial Sequence
  Synthetic Aptamer Sequence
aactacatggtatgtggtgaacttcaacgctatagagtgttatagtcaacgaacacatacgcagacgtacaatgtaccc

SEQ ID NO: 142 - DNA, 79 bp, Artificial Sequence
  Synthetic Aptamer Sequence
aactacatggtatgtggtgaactaagagatatatttcccaagtcccacagaaccccgatagaggacgtacaatgtaccc

SEQ ID NO: 143 - DNA, 79 bp, Artificial Sequence
  Synthetic Aptamer Sequence
aactacatggtatgtggtgaactgacgcgagtgcccaatgcataaagggagcgccctaaccgtgacgtacaatgtaccc

SEQ ID NO: 144 - DNA, 79 bp, Artificial Sequence
  Synthetic Aptamer Sequence
aactacatggtatgtggtgaactgtaagcaaacctccatccgcgataaataagctcgccccatgacgtacaatgtaccc

SEQ ID NO: 145 - DNA, 79 bp, Artificial Sequence
  Synthetic Aptamer Sequence
aactacatggtatgtggtgaactcagataagttccgtacatacagggccacagaggcaagatagacgtacaatgtaccc

SEQ ID NO: 146 - DNA, 79 bp, Artificial Sequence
  Synthetic Aptamer Sequence
aactacatggtatgtggtgaacttaagtcagcatcatacagtcatggatgtgccaagtcagatgacgtacaatgtaccc

SEQ ID NO: 147 - DNA, 79 bp, Artificial Sequence
  Synthetic Aptamer Sequence
aactacatggtatgtggtgaactactaggacacgaagacgcacagcgatcctaaagagccaacgacgtacaatgtaccc

SEQ ID NO: 148 - DNA, 79 bp, Artificial Sequence
  Synthetic Aptamer Sequence
aactacatggtatgtggtgaacttggcggaaaccaaccttgagcactgtaccatgttcgagcagacgtacaatgtaccc

SEQ ID NO: 149 - DNA, 79 bp, Artificial Sequence
  Synthetic Aptamer Sequence
aactacatggtatgtggtgaactcaaggcgataagaccatataaatggaatcacattaagatcgacgtacaatgtaccc

SEQ ID NO: 150 - DNA, 79 bp, Artificial Sequence
  Synthetic Aptamer Sequence
aactacatggtatgtggtgaactggccctgtataaagattcgactctgtcaaccacaaacctagacgtacaatgtaccc

SEQ ID NO: 151 - DNA, 79 bp, Artificial Sequence
  Synthetic Aptamer Sequence
aactacatggtatgtggtgaacttaggctcaatacttacctgatgacaggcgcccgcatcacagacgtacaatgtaccc

SEQ ID NO: 152 - DNA, 79 bp, Artificial Sequence
  Synthetic Aptamer Sequence
aactacatggtatgtggtgaactcggaagagctactcacaccgccaaggaccataagttctttgacgtacaatgtaccc

SEQ ID NO: 153 - DNA, 79 bp, Artificial Sequence
  Synthetic Aptamer Sequence
aactacatggtatgtggtgaactgaacacctattgacatgccaacagtggcggaccattagttgacgtacaatgtaccc

SEQ ID NO: 154 - DNA, 79 bp, Artificial Sequence
  Synthetic Aptamer Sequence
aactacatggtatgtggtgaactgtccgaaaagacgatcagacgaccatatgttaactgagctgacgtacaatgtaccc

SEQ ID NO: 155 - DNA, 79 bp, Artificial Sequence
  Synthetic Aptamer Sequence
aactacatggtatgtggtgaactctgctcaaataaacccatcaactgagaaagccaaatgttcgacgtacaatgtaccc

SEQ ID NO: 156 - DNA, 79 bp, Artificial Sequence
  Synthetic Aptamer Sequence
aactacatggtatgtggtgaacttcgggttgagaccacgtccatgcattgcgcacggttcagtgacgtacaatgtaccc

SEQ ID NO: 157 - DNA, 79 bp, Artificial Sequence
  Synthetic Aptamer Sequence
aactacatggtatgtggtgaactaaggcgggagatccttgttaacaggccacccaaccgagtagacgtacaatgtaccc

SEQ ID NO: 158 - DNA, 79 bp, Artificial Sequence
  Synthetic Aptamer Sequence
aactacatggtatgtggtgaacttccacatgatccgacttcagccgagcgttcctacgcagcagacgtacaatgtaccc

SEQ ID NO: 159 - DNA, 79 bp, Artificial Sequence
  Synthetic Aptamer Sequence
aactacatggtatgtggtgaactatccaaggaatcgaaaacctgtctccacgtgggcatctctgacgtacaatgtaccc

SEQ ID NO: 160 - DNA, 79 bp, Artificial Sequence
  Synthetic Aptamer Sequence
aactacatggtatgtggtgaactgtccattcttgaccactaacaatcccaccaggcgaggtgtgacgtacaatgtaccc

SEQ ID NO: 161 - DNA, 79 bp, Artificial Sequence
  Synthetic Aptamer Sequence
aactacatggtatgtggtgaactcaaccgatccgcgactcaaccgataaataagccatccacagacgtacaatgtaccc

SEQ ID NO: 162 - DNA, 79 bp, Artificial Sequence
  Synthetic Aptamer Sequence
aactacatggtatgtggtgaacttgtctatttgttcccaactaaacgtcagcaacacaccaacgacgtacaatgtaccc

SEQ ID NO: 163 - DNA, 79 bp, Artificial Sequence
  Synthetic Aptamer Sequence
aactacatggtatgtggtgaactaagaacagaatgtctgatccctggcgagaccaatatccatgacgtacaatgtaccc

SEQ ID NO: 164 - DNA, 79 bp, Artificial Sequence
  Synthetic Aptamer Sequence
aactacatggtatgtggtgaacttaaccaacgccacactgacatgcgccattatcaaggagtagacgtacaatgtaccc

SEQ ID NO: 165 - DNA, 79 bp, Artificial Sequence
  Synthetic Aptamer Sequence
aactacatggtatgtggtgaactgtctgatgatctggtctcgattcagtagataacagccaccgacgtacaatgtaccc

SEQ ID NO: 166 - DNA, 79 bp, Artificial Sequence
  Synthetic Aptamer Sequence
aactacatggtatgtggtgaactcggcacagaactaccctccaacaagagagcgcctttatcagacgtacaatgtaccc

SEQ ID NO: 167 - DNA, 79 bp, Artificial Sequence
  Synthetic Aptamer Sequence
aactacatggtatgtggtgaactcccactcgctcagtcgggaagaccggtggtaggagccttagacgtacaatgtaccc

SEQ ID NO: 168 - DNA, 79 bp, Artificial Sequence
  Synthetic Aptamer Sequence
aactacatggtatgtggtgaactggattgggatatcagaatttaatcagctcacaagcaaaccgacgtacaatgtaccc

SEQ ID NO: 169 - DNA, 79 bp, Artificial Sequence
  Synthetic Aptamer Sequence
aactacatggtatgtggtgaactcagtcagtctaaggtaacacaacttgcatggatgaacaccgacgtacaatgtaccc

SEQ ID NO: 170 - DNA, 79 bp, Artificial Sequence
  Synthetic Aptamer Sequence
aactacatggtatgtggtgaactgcagctaaccactgaactggtcgtagcccgcaacaacagagacgtacaatgtaccc

SEQ ID NO: 171 - DNA, 79 bp, Artificial Sequence
  Synthetic Aptamer Sequence
aactacatggtatgtggtgaactcgagtaagtcaaacgctcaccatcttacaaggcgcatctagacgtacaatgtaccc

SEQ ID NO: 172 - DNA, 79 bp, Artificial Sequence
  Synthetic Aptamer Sequence
aactacatggtatgtggtgaactgctcatacactgcaaggaagtagagcggtgtaacagtcccgacgtacaatgtaccc

SEQ ID NO: 173 - DNA, 79 bp, Artificial Sequence
  Synthetic Aptamer Sequence
aactacatggtatgtggtgaactaggcgccacatggcaataacggtccgctatagtcgtattagacgtacaatgtaccc

SEQ ID NO: 174 - DNA, 79 bp, Artificial Sequence
  Synthetic Aptamer Sequence
aactacatggtatgtggtgaactcggaaggaaccaagttaatctttgaactggtccgagacttgacgtacaatgtaccc

SEQ ID NO: 175 - DNA, 79 bp, Artificial Sequence
  Synthetic Aptamer Sequence
aactacatggtatgtggtgaactgctcgtatacaaactatccttgtccgccacttgttgcaccgacgtacaatgtaccc

SEQ ID NO: 176 - DNA, 79 bp, Artificial Sequence
  Synthetic Aptamer Sequence
aactacatggtatgtggtgaactatcggttgtttaccacggaaactgcgcagtttcgaaaggcgacgtacaatgtaccc

SEQ ID NO: 177 - DNA, 79 bp, Artificial Sequence
  Synthetic Aptamer Sequence
aactacatggtatgtggtgaactactcaacaattcagacagcacgtgttaagtatattgcatagacgtacaatgtaccc

SEQ ID NO: 178 - DNA, 79 bp, Artificial Sequence
  Synthetic Aptamer Sequence
aactacatggtatgtggtgaactaagacaggcgaacctgaagtcaagcaaccacatgcccgaggacgtacaatgtaccc

SEQ ID NO: 179 - DNA, 79 bp, Artificial Sequence
  Synthetic Aptamer Sequence
aactacatggtatgtggtgaactaggatcaatgtcctgaagccagtcgttggccgtgaatcaagacgtacaatgtaccc

SEQ ID NO: 180 - DNA, 79 bp, Artificial Sequence
  Synthetic Aptamer Sequence
aactacatggtatgtggtgaacttggcgtaaaagttagaaccatcattgctccacgctacatggacgtacaatgtaccc

SEQ ID NO: 181 - DNA, 79 bp, Artificial Sequence
  Synthetic Aptamer Sequence
aactacatggtatgtggtgaacttgccattcacgtaccgttagggccgtccaaatccacgtaggacgtacaatgtaccc

SEQ ID NO: 182 - DNA, 79 bp, Artificial Sequence
  Synthetic Aptamer Sequence
aactacatggtatgtggtgaactaaacgcacatacgatcctgcgccgaagatcaaggtaaggagacgtacaatgtaccc

SEQ ID NO: 183 - DNA, 79 bp, Artificial Sequence
  Synthetic Aptamer Sequence
aactacatggtatgtggtgaactgttccaaacacacaacatggcgtcatgtcacaattcaattgacgtacaatgtaccc

SEQ ID NO: 184 - DNA, 79 bp, Artificial Sequence
  Synthetic Aptamer Sequence
aactacatggtatgtggtgaactgttacttggtagagccaaggctttacaaagttcgaactcagacgtacaatgtaccc

SEQ ID NO: 185 - DNA, 79 bp, Artificial Sequence
  Synthetic Aptamer Sequence
aactacatggtatgtggtgaactgtataacgaaatccagccacgtactgcgatacgcgaaaatgacgtacaatgtaccc

SEQ ID NO: 186 - DNA, 79 bp, Artificial Sequence
  Synthetic Aptamer Sequence
aactacatggtatgtggtgaactctctcagtgaagcctggaatagaataccacgcacgcggtcgacgtacaatgtaccc

SEQ ID NO: 187 - DNA, 79 bp, Artificial Sequence
  Synthetic Aptamer Sequence
aactacatggtatgtggtgaactcaacgagagtgggagcacctacagacgcatgggcaaatgagacgtacaatgtaccc

SEQ ID NO: 188 - DNA, 79 bp, Artificial Sequence
  Synthetic Aptamer Sequence
aactacatggtatgtggtgaacttaaaggcataggacatgctcaggaggtcaccgccaaaccagacgtacaatgtaccc

SEQ ID NO: 189 - DNA, 79 bp, Artificial Sequence
  Synthetic Aptamer Sequence
aactacatggtatgtggtgaactactcgaagcgttccaattttggagtcttctgacaccagccgacgtacaatgtaccc

SEQ ID NO: 190 - DNA, 79 bp, Artificial Sequence
  Synthetic Aptamer Sequence
aactacatggtatgtggtgaactcagagtaaagtctcgcaagtgcaccgctaatctacccgcagacgtacaatgtaccc

SEQ ID NO: 191 - DNA, 79 bp, Artificial Sequence
  Synthetic Aptamer Sequence
aactacatggtatgtggtgaactcgaaaattcatcccacaggctggtggcacgactagaacgagacgtacaatgtaccc

SEQ ID NO: 192 - DNA, 79 bp, Artificial Sequence
  Synthetic Aptamer Sequence
aactacatggtatgtggtgaactttccaaacaattcagagatggaccacataaaccccaatgcgacgtacaatgtaccc

SEQ ID NO: 193 - DNA, 79 bp, Artificial Sequence
  Synthetic Aptamer Sequence
aactacatggtatgtggtgaactatcatcacaccgtggaaggattgagtccgacggagatcacgacgtacaatgtaccc

SEQ ID NO: 194 - DNA, 79 bp, Artificial Sequence
  Synthetic Aptamer Sequence
aactacatggtatgtggtgaactttccatctataactgtcaaaagcacacctcgactacccgagacgtacaatgtaccc

SEQ ID NO: 195 - DNA, 79 bp, Artificial Sequence
  Synthetic Aptamer Sequence
aactacatggtatgtggtgaactacatggcgagacgatgatgagtgcaccagatccattagatgacgtacaatgtaccc

SEQ ID NO: 196 - DNA, 79 bp, Artificial Sequence
  Synthetic Aptamer Sequence
aactacatggtatgtggtgaactagagtctaagaataggttaaacctggtcaagctcagcccagacgtacaatgtaccc

SEQ ID NO: 197 - DNA, 79 bp, Artificial Sequence
  Synthetic Aptamer Sequence
aactacatggtatgtggtgaactagccaaatccttccctgtcgccagagtgattggttcccaagacgtacaatgtaccc

SEQ ID NO: 198 - DNA, 79 bp, Artificial Sequence
  Synthetic Aptamer Sequence
aactacatggtatgtggtgaactaagcacggataatgcgtcaaagtgaggacaagccaagaatgacgtacaatgtaccc

SEQ ID NO: 199 - DNA, 79 bp, Artificial Sequence
  Synthetic Aptamer Sequence
aactacatggtatgtggtgaactgcaaagtatttccaagcaccgtagtagggaatcaatgtgagacgtacaatgtaccc

SEQ ID NO: 200 - DNA, 79 bp, Artificial Sequence
  Synthetic Aptamer Sequence
aactacatggtatgtggtgaacttgccattaatagcgcggctagaacacatttcacacacaacgacgtacaatgtaccc

SEQ ID NO: 201 - DNA, 10 bp, Artificial Sequence
  Synthetic Aptamer Sequence
  misc_feature (1)..(1): n is a, c, g, or t
  misc_feature (9)..(10): n is a, c, g, or t
ncgagcacnn

SEQ ID NO: 202 - DNA, 10 bp, Artificial Sequence
  Synthetic Aptamer Sequence
  misc_feature (1)..(2): n is a, c, g, or t
  misc_feature (9)..(10): n is a, c, g, or t
nnacaagtnn

SEQ ID NO: 203 - DNA, 10 bp, Artificial Sequence
  Synthetic Aptamer Sequence
aaaccacgac

SEQ ID NO: 204 - DNA, 10 bp, Artificial Sequence
  Synthetic Aptamer Sequence
  misc_feature (1)..(2): n is a, c, g, or t
  misc_feature (9)..(10): n is a, c, g, or t
nnattcacnn

SEQ ID NO: 205 - DNA, 10 bp, Artificial Sequence
  Synthetic Aptamer Sequence
  misc_feature (1)..(1): n is a, c, g, or t
  misc_feature (9)..(10): n is a, c, g, or t
nacaccgann

SEQ ID NO: 206 - DNA, 10 bp, Artificial Sequence
  Synthetic Aptamer Sequence
  misc_feature (1)..(1): n is a, c, g, or t
  misc_feature (10)..(10): n is a, c, g, or t
ngacaacagn

SEQ ID NO: 207 - DNA, 15 bp, Artificial Sequence
  Synthetic Aptamer Sequence
  misc_feature (8)..(8): n is a, c, g, or t
acaccgangacaaca

SEQ ID NO: 208 - DNA, 10 bp, Artificial Sequence
  Synthetic Aptamer Sequence
  misc_feature (1)..(1): n is a, c, g, or t
  misc_feature (10)..(10): n is a, c, g, or t
ngcagaacan

SEQ ID NO: 209 - DNA, 10 bp, Artificial Sequence
  Synthetic Aptamer Sequence
  misc_feature (1)..(1): n is a, c, g, or t
  misc_feature (9)..(10): n is a, c, g, or t
naacatgann

SEQ ID NO: 210 - DNA, 18 bp, Artificial Sequence
  Synthetic Aptamer Sequence
tgaccaaaagaggaaagg

SEQ ID NO: 211 - DNA, 11 bp, Artificial Sequence
  Synthetic Aptamer Sequence
aagaggaaagg

SEQ ID NO: 212 - DNA, 26 bp, Artificial Sequence
  Synthetic Aptamer Sequence
gcagaacatgaccaaaagaggaaagg

SEQ ID NO: 213 - DNA, 10 bp, Artificial Sequence
  Synthetic Aptamer Sequence
  misc_feature (1)..(1): n is a, c, g, or t
  misc_feature (10)..(10): n is a, c, g, or t
naaccacaan

SEQ ID NO: 214 - DNA, 39 bp, Artificial Sequence
  Synthetic Aptamer Sequence
aaagtgaactgaatatggattacaagtttcagatcgaaa

SEQ ID NO: 215 - DNA, 51 bp, Artificial Sequence
  Synthetic Aptamer Sequence
aaactacatggtatgtggtgaaccaaaggatcgagcactcccattcagaaa

SEQ ID NO: 216 - DNA, 36 bp, Artificial Sequence
  Synthetic Aptamer Sequence
ctacatggtatgtggaaaccacgacccaggatgtgc

SEQ ID NO: 217 - DNA, 37 bp, Artificial Sequence
  Synthetic Aptamer Sequence
aaagtggtgaactaggggaaccttagtaaaccacaaa

SEQ ID NO: 218 - DNA, 41 bp, Artificial Sequence
  Synthetic Aptamer Sequence
aaaggtgaacttagtgggatttattcactatgtacaccgat

SEQ ID NO: 219 - DNA, 25 bp, Artificial Sequence
  Synthetic Aptamer Sequence
aattagtgggatttattcactataa

SEQ ID NO: 220 - DNA, 40 bp, Artificial Sequence
  Synthetic Aptamer Sequence
actgcagaacatgaccaaaagaggaaaggtatagctgcta